(19) United States
(12) Patent Application Publication
(10) Pub. No.: US 2024/0109925 A1
(43) Pub. Date: Apr. 4, 2024

(54) Enhanced Anti-Proliferative and Antitumor Immune Effects of Mitochondria-Targeted Hydroxyurea

(71) Applicants: The Medical College of Wisconsin, Inc., Milwaukee, WI (US); Aix-Marseille Universite, Marseille (FR)

(72) Inventors: Balaraman Kalyanaraman, Milwaukee, WI (US); Gang Cheng, Milwaukee, WI (US); Micael Joel Hardy, Nimes (FR)

(21) Appl. No.: 18/262,985

(22) PCT Filed: Jan. 26, 2022
(86) PCT No.: PCT/US2022/013896

§ 371 Date: Jul. 26, 2023

Related U.S. Application Data
(60) Provisional application No. 63/141,802, filed on Jan. 26, 2021.

(51) Int. Cl. C07F 9/54; A61K 31/475; A61K 31/519; A61P 35/00

(52) U.S. Cl. CPC C07F 9/5442; A61K 31/475; A61K 31/519; A61P 35/00

(57) ABSTRACT

In the present invention, the inventors provide substituted the hydroxyl group in hydroxyurea (HU) with a triphenylphosphonium (TPP) cation attached to an alkyl group with different chain lengths, generating a new class of mitochondria-targeted hydroxyurea compounds (Mito-HUs). Elongating the alkyl side chain length increased the hydrophobicity of Mito-HUs, and correlated with increased inhibition of oxidative phosphorylation, and antiproliferative effects in tumor cells.

### CROSS-REFERENCE TO RELATED APPLICATIONS

This application claims the benefit of priority under 35 U.S.C. § 119(e) of U.S. Provisional Application No. 63/141,802, filed Jan. 26, 2021, the contents of which is incorporated herein by reference in its entirety.

### STATEMENT REGARDING FEDERALLY SPONSORED RESEARCH OR DEVELOPMENT

N/A

### FIELD OF THE INVENTION

This invention relates generally to mitochondria-targeting hydroxyurea (HU) and methods of using the compounds to treat cancer and enhance the immune response to cancer

### BACKGROUND

Hydroxyurea (HU), an FDA-approved drug for treating sickle cell disease, is used as an antitumor drug alone and together with conventional chemotherapeutics or radiation therapy. HU is used primarily to treat myeloproliferative diseases because it inhibits the enzyme ribonucleotide reductase, which is involved in DNA synthesis. The hydroxyl group in HU is considered critical for its antiproliferative and chemotherapeutic effects.

### BRIEF SUMMARY

The present invention provides, in one aspect, a mito-hydroxyurea compound of one of the formulas:

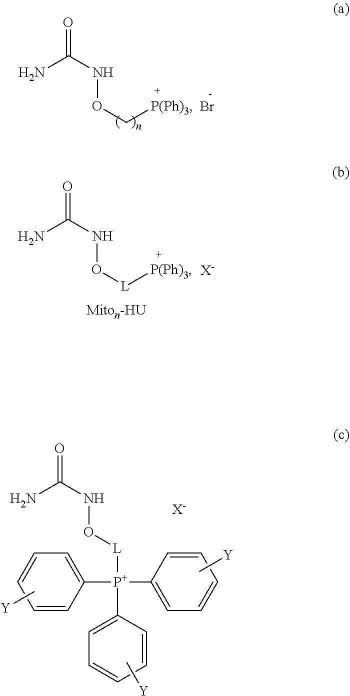

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl, substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid.

In another aspect, the disclosure provides a mito-hydroxyurea compound of (c), wherein the compound is

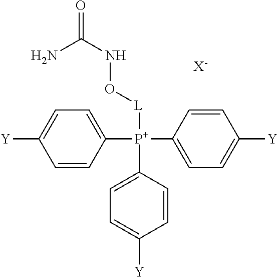

wherein the Y is para.

In a further aspect, the disclosure provides the following compounds:

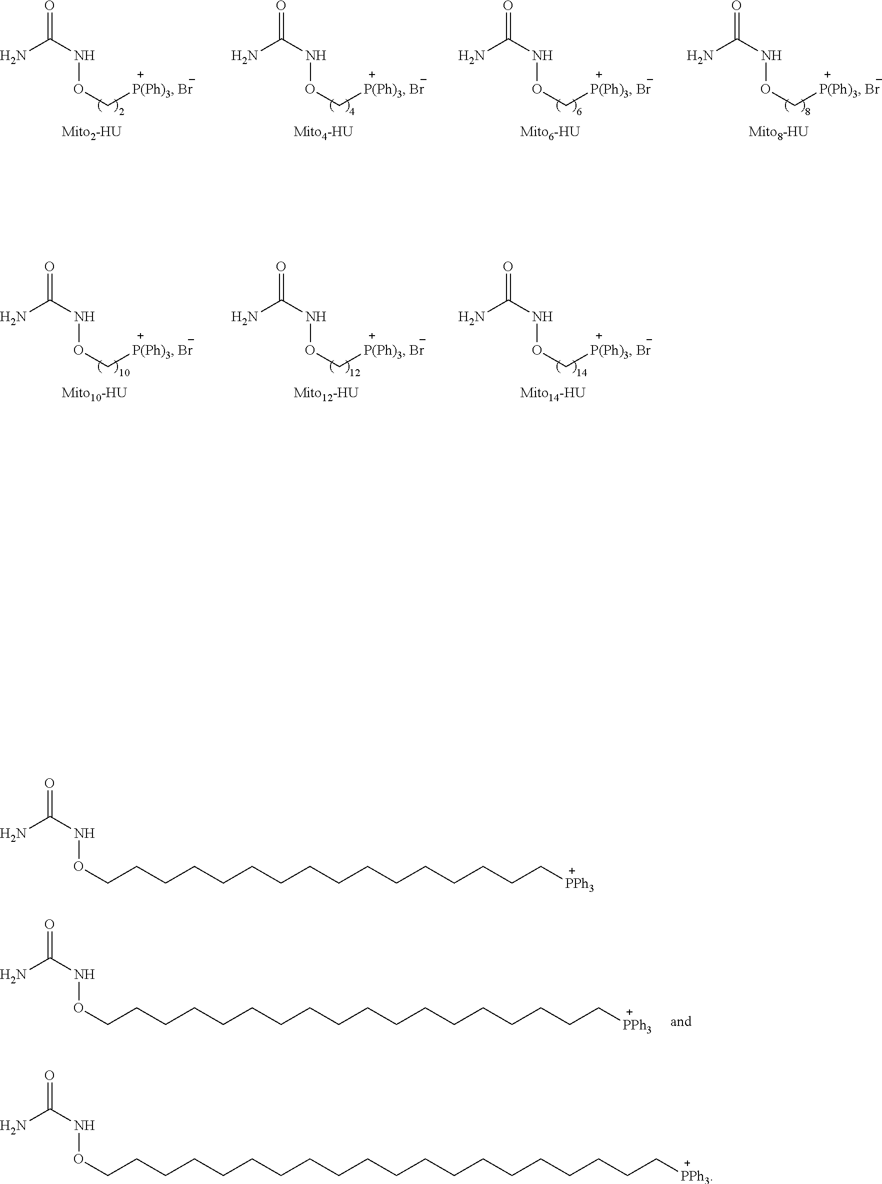

In yet another aspect, the disclosure provides a composition comprising the mito-hydroxyurea compound described herein and a pharmaceutically acceptable carrier.

In another aspect, the disclosure provides a method of treating cancer in a subject in need thereof, the method comprising administering a therapeutically effective amount of the compound described herein to the subject to treat the cancer. In some embodiments, the composition is administered with a therapeutically effective amount of a monocarboxylate transporter 1 (MCT-1) inhibitor is also administered to the subject.

In a further aspect, the disclosure provides a method of inhibiting mitochondrial complex I and complex III in a subject in need thereof, the method comprising administering a therapeutically effective amount of the compound described herein to the subject in order to inhibit, mitochondrial complex I, III or both in a subject.

In another aspect, the disclosure provides a method of increasing an immune response to cancer cells in a subject in need thereof, the method comprising administering a therapeutically effective amount of the compound described herein to the subject in order to increase an immune response to the cancer cells.

In yet another embodiment, the disclosure provides a method of reducing the number of immunosuppressive cells in a subject in need thereof, the method comprising administering a therapeutically effective amount of the compound described herein to the subject in order to reduce the number of immunosuppressive cells in the subject.

In another aspect, the disclosure provides a method of potentiating radiation therapy in a subject in need thereof, the method comprising administering a compound or the composition described herein to a subject and treating the subject with radiation therapy.

In yet another aspect, the disclosure provides a method of inhibiting proliferation of a cancer cell, the method comprising contacting a cancer cell with an effective amount of the compound described herein in order to inhibit proliferation of the cancer cell.

In yet another aspect, the disclosure provides a method of increasing the production of interferon-γ (IFN-γ) in a T cell, the method comprising contacting the cell with an effective amount of the compound or the composition described herein to increase production of interferon-γ.

Other aspects are described herein.

### BRIEF DESCRIPTION OF THE DRAWINGS

FIG. 1. Effects of HU and Mito-HUs on proliferation of HCT116 human colon cancer cells (A) Effects of HU and Mito-HUs on the proliferation of HCT116 cells were monitored in the IncuCyte Live-Cell Analysis system. The IncuCyte Analyzer provides real-time updates on cell confluence, based on segmentation of high definition-phase contrast images. Representative cell images were shown as segmentation mask illustrated in brown when control cells reached 90% confluence (vertical solid black line). (B) The IC50 values were determined at the point at which control cells reached ˜90% confluence (vertical solid black line). Relative cell confluence (control is taken as 100%) is plotted against concentration. Dashed lines represent the fitting curves used to determine the IC50 values as indicated. Data shown are the means & SD.

FIG. 2. Effects of HU and Mito-HUs on the oxygen consumption by mitochondrial complexes I and III (A) Dose-dependent effects of HU, Mito10-HU, Mito12-HU, Mito14-HU, Mito16-HU, and Mito20-HU on complex I- and complex III-dependent oxygen consumption were measured in HCT116 cells. HCT116 cells were treated with HU or Mito-HUs as indicated for 24 hr. Mitochondrial complex I activity (left) and complex III activity (right) were monitored by a Seahorse XF-96 analyzer. Either rotenone (complex I inhibitor) or antimycin A (complex III inhibitor) was acutely added and OCR assayed immediately. (B) The mitochondrial complex I (left) and III (right) dependent oxygen consumption (calculated as rotenone or antimycin A inhibitable OCR) are plotted against concentration of HU and Mito-HUs. Dashed lines represent the fitting curves used for determination of the IC50 values. Data shown are the means G SD.

FIG. 3. Relationship between Mito-HUs and inhibitions of cell proliferation or complex I/III activities in HCT116 cells The chain lengths (left) and log Ps (right) of the Mito-HUs are plotted against the fold changes (potency) compared to the IC50 values of Mito10-HU for (A) cell proliferation, (B) mitochondrial complex I, and (C) complex III activities. The IC50 values used to calculate the fold changes are from FIGS. 1 and 2, respectively. Data shown are the means & SD.

FIG. 4. Effects of Mito10-HU on in vitro differentiation of CD4 T cells (A) The gating strategy reflects data from one DMSO control sample. First, the total population of cells was gated (FSC-A vs SSC-A). Then, single cells were gated (SSC-W vs SSC-H). Within the single cell population, we gated on the live CD4 T cells using a fixable LIVE/DEAD stain (CD4 vs DEAD). Within the live CD4 T cells we gated on CD25+FOXP3+Tregs (FOXP3 vs CD25), as well as IFNg-YFP+CD4 T cells (IFNg-YFP vs CD4). (B) Live CD4 T cells were gated within the single cell populations, at varying concentrations of Mito10-HU, along with DMSO controls (B, top row). CD25+FOXP3+Tregs (B, middle row) and IFNg-YFP+CD4 T cells (B, bottom row) were gated within the live CD4 T cell populations, at varying concentrations of Mito10-HU, along with DMSO controls. (C) Effects of Mito10-HU on live CD4+T cells within the lymphocyte gate (C, left), the frequency of FOXP3+CD25+cells within the live CD4+T cells (C, middle), and Teff function is shown as the frequency of IFNg-YFP positive cells within the live CD4+T cells (C, right). Data shown are the means & SD, n=2 per treatment group. *p<0.05, **p<0.01 vs control.

FIG. 5. In vitro differentiation of MDSCs and suppressive neutrophils in the presence of different concentrations of Mito14-HU and Mito-HUs with varying hydrophobicities (A) Scheme of differentiation of MDSCs. (B) Representative flow cytometry contour plots of in vitro differentiation of MDSCs under varying concentrations of Mito14-HU using FlowJo Software (FlowJo Software, 2019). (C) Summary data depicting distribution of MDSCs after three days treatment with Mito14-HU generated from panel (B). (D and E) (D) Dose-dependent effects of Mito-HUs with different side chain lengths on M-MDSC and (E) suppressive neutrophils. Dashed lines represent the fitting curves used for determination of the IC50 values. Data shown are the means & SD.

FIG. 6. Effects of decyl-HU on proliferation of HCT116 human colon cancer cells HCT116 cells treated with decyl-HU were monitored in the IncuCyte Live-Cell Analysis system. Cell proliferation was monitored in real-time with the continuous presence of indicated treatments. Data shown are the means±SD.

FIG. 7. Effects of HU and Mito10-HU on proliferation of MiaPaCa-2 human pancreatic cancer cells; MiaPaCa-2 cells treated with HU or Mito10-HU were monitored in the IncuCyte Live-Cell Analysis system. Cell proliferation was monitored in real-time with the continuous presence of indicated treatments until the end of each experiment. Data shown are the means±SD.

FIG. 8. Effects of Mito10-HUNTPP+ and Mito10-HU on proliferation of HCT116 human colon cancer cells HCT116 cells were treated with Mito10-HUNTPP+ (amino hydrogen in HU was substituted with a 10-carbon aliphatic chain attached to TPP+). (A) Cell proliferation was monitored in real-time in the IncuCyte Live-Cell Analysis system. (B) The IC50 values obtained with Mito10-HUNTPP+ were compared with the cell confluence obtained with Mito10-HU in HCT116 cells. The IC50 values were determined at the point at which untreated cells reached ˜90% confluence. Cell confluence of the percentage of control is plotted against concentration. Dashed lines represent the fitting curves used to determine the IC50 values as indicated. Data shown are the means±SD.

FIG. 9. Effects of Mito10-HU and Mito10-HUtriMe on proliferation of HCT116 human colon cancer cells HCT116 cells were treated with Mito10-HU and Mito10-HUtriMe. (A) Cell proliferation was monitored in real-time in the IncuCyte Live-Cell Analysis system. (B) The IC50 values obtained with Mito10-HUtriMe were compared with the cell confluence obtained with Mito10-HU and HU in HCT116 cells. The IC50 values were determined at the point at which untreated cells reached ˜90% confluence. Cell confluence of the percentage of control is plotted against concentration. Dashed lines represent the fitting curves used to determine the IC50 values as indicated. Data shown are the means±SD.

FIG. 10. The SYTOX Green assay monitoring the cytotoxicity of Mito-HUs in HCT116 cells. HCT116 cells were treated with Mito-HUs at the indicated concentrations (IC50 values from FIG. 1) for 24 h and 48 h. Cell death with strong green fluorescence intensity was monitored with the IncuCyte Live-Cell Analysis system by SYTOX Green staining. The corresponding representative fluorescence images are shown in the left (24 h) and middle panel (48 h). The right panel shows the total cell number after all cells were permeabilized with digitonin (120 μM).

FIG. 11. Effects of Mito10-HU on the activity of mitochondrial complexes I and III in MiaPaCa-2 human pancreatic cancer cells. (A) Dose-dependent effect of Mito10-HU on complex I- and complex III-dependent oxygen consumption was measured in MiaPaCa-2 cells. MiaPaCa-2 cells were treated with Mito10-HU for 24 h. Mitochondrial complex I activity (left) and complex III activity (right) were monitored by Seahorse XF-96 Extracellular Flux Analyzer. Either Rot (complex I inhibitor) or AA (complex III inhibitor) was acutely added and OCR assayed immediately as described in FIG. 3. (B) The mitochondrial complexes I and III-dependent oxygen consumptions (calculated as Rot or AA inhibitable OCR) are plotted against concentration of Mito10-HU. Dashed lines represent the fitting curves used for determination of the IC50 values. Data shown are the means±SD.

FIG. 12. Effects of Mito10-HU and Mito10-HUtriMe on the activity of mitochondrial complexes I and III in HCT116 human colon cancer cells. (A) Dose-dependent effect of Mito10-HUtriMe on complex I- and complex III-dependent oxygen consumption was measured in HCT116 cells. HCT116 cells were treated with Mito10-HUtriMe for 24 h. Mitochondrial complex I activity (left) and complex III activity (right) were monitored by Seahorse XF-96 Extracellular Flux Analyzer. Either Rot (complex I inhibitor) or AA (complex III inhibitor) was acutely added and OCR assayed immediately, as described in FIG. 3. (B) The mitochondrial complex I—(left) and complex III—(right) dependent oxygen consumption (calculated as Rot or AA inhibitable OCR) are plotted against concentration of HU and Mito-HUs. Dashed lines represent the fitting curves used for determination of the IC50 values. Data shown are the means±SD.

FIG. 13. Relationship between alkyl chain length and relative hydrophobicities in Mito-HUs; related to FIG. 33 and Syntheses of Mito-HUs in Table 1. The alkyl side chain lengths of the Mito-HUs are plotted against the log P value (from FIG. 33, relative hydrophobicities) of the Mito-HUs.

FIG. 14. Relationship between chemical structures of Mito-HUs and inhibitions of mitochondrial complex III activities in HCT116 cells; related to FIG. 33, FIG. 2, and Syntheses of Mito-HUs and Mitochondrial oxygen consumption measurements in the STAR Methods. The alkyl side chain length (left) and log P (right) of the Mito-HUs are plotted against the fold changes (potency) compared to the IC50 values of Mito10-HU on mitochondrial complex III activities. There is a good linear fit for Mito-HU chain lengths up to 14, with fitting R2=0.999. The IC50 values are from FIG. 2.

FIG. 15. Synergistic inhibition of cell proliferation by Mito10-HU and syrosingopine in HCT116 cells; related to FIGS. 1 and 2 and Cell proliferation measurements in the STAR Methods. (A) HCT116 cells were treated with Mito10-HU (5 μM) or syrosingopine (7.5 μM) independently and together, and cell growth was monitored continuously. Data shown are the mean±SD (n=5). Representative cell images are shown as a segmentation mask illustrated in brown when control cells reached ˜90% confluence (vertical dashed line). **p<0.01 vs control. ##p<0.01 vs Mito10-HU alone. (B) Cell confluence (control cells reached ˜90% confluence) is plotted against concentration for the synergistic calculation. Panel B shows the combination index-fraction affected plots. Fraction affected parameter is used as a measure of the drug(s) efficiency, with a value of 0 indicating complete inhibition of cell confluence and a value of 1 indicating the lack of effect on cell confluence. Data shown are the means±SD.

FIG. 16. Effects of Mito-HUs with varying hydrophobicities on in vitro differentiation of CD4 T cells; related to FIG. 4 and Immunoregulatory measurements in the STAR Methods. Differentiation of CD4 T cells treated with Mito-HUs with varying hydrophobicities (A, HU; B, Mito12-HU; C, Mito14-HU; D, Mito16-HU; and E, Mito20-HU) under the same experimental conditions as detailed for FIG. 4.

Effects on live CD4+ T cells within the lymphocyte gate (left), the frequency of FoxP3+CD25+cells within the live CD4+T cells (middle), and Teff function is shown as the frequency of IFNγ-YFP positive cells within the live CD4+T cells (right). Data shown are the means±SD, n=2 per treatment group. *p<0.05, **p<0.01 vs control.

FIG. 17. NMR Spectra of Mito-HU_{NHTPP}

FIG. 18. NMR Spectra of Mito_{triMe}-Br

FIG. 19. NMR Spectra of Mito₁₀-HU_{triMe}

FIG. 20. NMR Spectra of Mito₄-HU

FIG. 21. NMR Spectra of Mito₁₀-HU

FIG. 22. NMR Spectra of Mito₁₂-HU

FIG. 23. NMR Spectra of dibromoalcane

FIG. 24. NMR Spectra of Mito₁₄-Br

FIG. 25. NMR Spectra of Mito₁₄-HU

FIG. 26. NMR Spectra of Mito₁₆-Br

FIG. 27. NMR Spectra of Mito₁₆-HU

FIG. 28. NMR Spectra of Mito₂₀-HU

FIG. 29. NMR Spectra of Decyl-HU

FIG. 30. Synthesis of HUs; related to Syntheses of Mito-HUs in the STAR Methods. Reagents and conditions. i, (10-aminodecyl)-triphenylphosphonium bromide, CDI, hydroxyurea, reflux, 12 h, 23%; ii, (10-bromodecyl)-tris-p-tolylphosphonium (Mito_{triMe}-Br), potassium carbonate, DMF, 45° C., 4 h, (57%); iii, bromodecane, potassium carbonate, DMF, 70° C., 4 h, (31%); iv, potassium carbonate, DMF, Mitoₙ-Br, 45° C., 4 h, (38-82%).

FIG. 31. Synthesis of Mito_{triMe}-Br; related to Syntheses of Mito-HUs in the STAR Methods. Reagents and conditions: i, tri-p-tolylphosphine, neat, 80° C., 60%.

FIG. 32. Synthesis of Miton-Br variants; related to Syntheses of Mito-HUs in the STAR Methods. Reagents and conditions: i, HBr (48%), anhydride acetic, reflux, 24 h, (86-100%). ii, PPh3, neat, 90° C., 6-12 h, (53-80%).

FIG. 33. Calculated partition coefficients and relative hydrophobic regions in HU and Mito-HUs

FIG. 34. Calculated partition coefficients and relative hydrophobic regions in Mito-HUs with substituted TPP

FIG. 35. Synergistic inhibition of cell proliferation by Mito₁₀-HU and AZD-3965 in HCT-116 cells. (A) HCT-116 cells were treated with Mito₁₀-HU (5 μM) or AZD-3965 (100 μM) independently and together, and cell growth was monitored continuously. Data shown are the mean±SD (n=5). Representative cell images are shown as a segmentation mask illustrated in brown when control cells reached ˜90% confluence (vertical dashed line). **p<0.01 vs control. **p<0.01 vs Mito₁₀-HU alone. (B) Cell confluence (control cells reached ˜90% confluence) is plotted against concentration for the synergistic calculation. Panel B shows the combination index-fraction affected plots. Fraction affected parameter is used as a measure of the efficiency of the drug(s), with a value of 0 indicating complete inhibition of cell confluence and a value of 1 indicating the lack of effect on cell confluence. Data shown are the mean±SD.

### DETAILED DESCRIPTION

In the present invention, the inventors substituted the hydroxyl group in hydroxyurea (HU) with a triphenylphosphonium (TPP) cation attached to an alkyl group with different chain lengths, generating a new class of mitochondria-targeted hydroxyurea compounds (Mito-HUs). Elongating the alkyl side chain length increased the hydrophobicity of Mito-HUs, and correlated with increased inhibition of oxidative phosphorylation, and antiproliferative effects in tumor cells.

To the inventors' surprise, Mito-HU was much more potent than HU in inhibiting proliferation of tumor cells (e.g., AML and MiaPaCa-2 pancreatic cancer cells). Furthermore, the Inventors also discovered that Mito-HU inhibits both mitochondrial complex I and complex III activities at low micromolar concentrations. Moreover, both mitochondrial complex I- and complex III-induced oxygen consumption decreased with the increasing hydrophobicity of Mito-HUs. The more hydrophobic Mito-HUs also potently inhibited monocytic myeloid-derived suppressor cells, suppressive neutrophils, and stimulated T cell effector function, demonstrating that the disclosed mito-HU compounds likely have antitumor immunomodulatory effects.

Previous reports suggest that replacing the ⁻OH group in HU with an alkyl group inhibits its antiproliferative effect due to lack of a tyrosyl or cysteinyl radical scavenging mechanism. However, replacing the ⁻OH group with alkyl substituted TPP⁺ potently stimulates the antiproliferative mechanism that is dependent on the alkyl side chain length; Mito₄-HU (log P=4.0) did not inhibit the proliferation of MiaPaCa-2 cells, whereas Mito₁₀-HU (log P=6.7), Mito₁₂-HU (log P=7.6), and Mito₁₆-HU (log P=9.4) with increasing hydrophobicity exhibited increasing antiproliferative potencies. This suggests that replacement of the ⁻OH group with the hydrophobic cationic substituent targeting mitochondria alters the antitumor action of HU.

### Mito-HU

In some aspects of the current disclosure, mitochondrial targeted hydroxyurea compounds (mito-HUs) are provided. In some embodiments, the mito-HU compounds have a formula selected from:

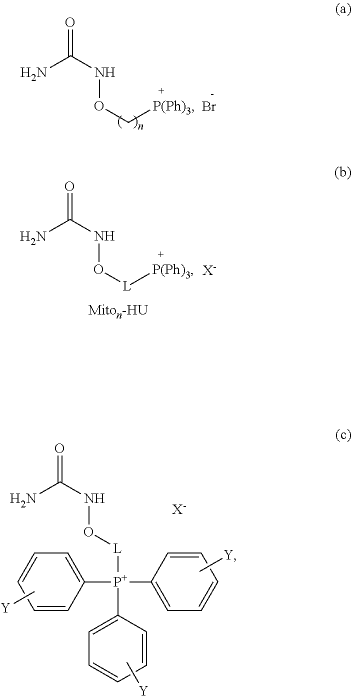

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
   X⁻ is selected from halogen, 2,2,2-trifluoroacetic acid (TFA), HO⁻, RCOO⁻, and acetic acid.

In one embodiment, the compound is compound (c)

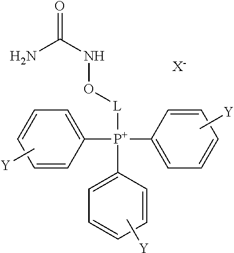

wherein Y is ortho, meta or para, and wherein Y is independently selected from —H, —CF₃, methyl (Me), Cl, OMe, C(O)CH₃, NO₂, N(Me)₂.

In one embodiment, compound (c) is:

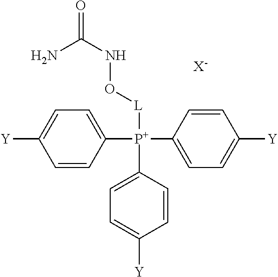

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
   X⁻ is selected from halogen, 2,2,2-trifluoroacetic acid (TFA), HO⁻, RCOO⁻.

As discussed above, the Inventors discovered that the increasingly hydrophobic aliphatic linker correlated with increased activity of mito-HU compounds. Therefore, in some embodiments, n=2, 4, 6, 8, 10, 12, 14, 16, 18, 20, or more.

In some embodiments, the mito-hydroxyurea compound is selected from

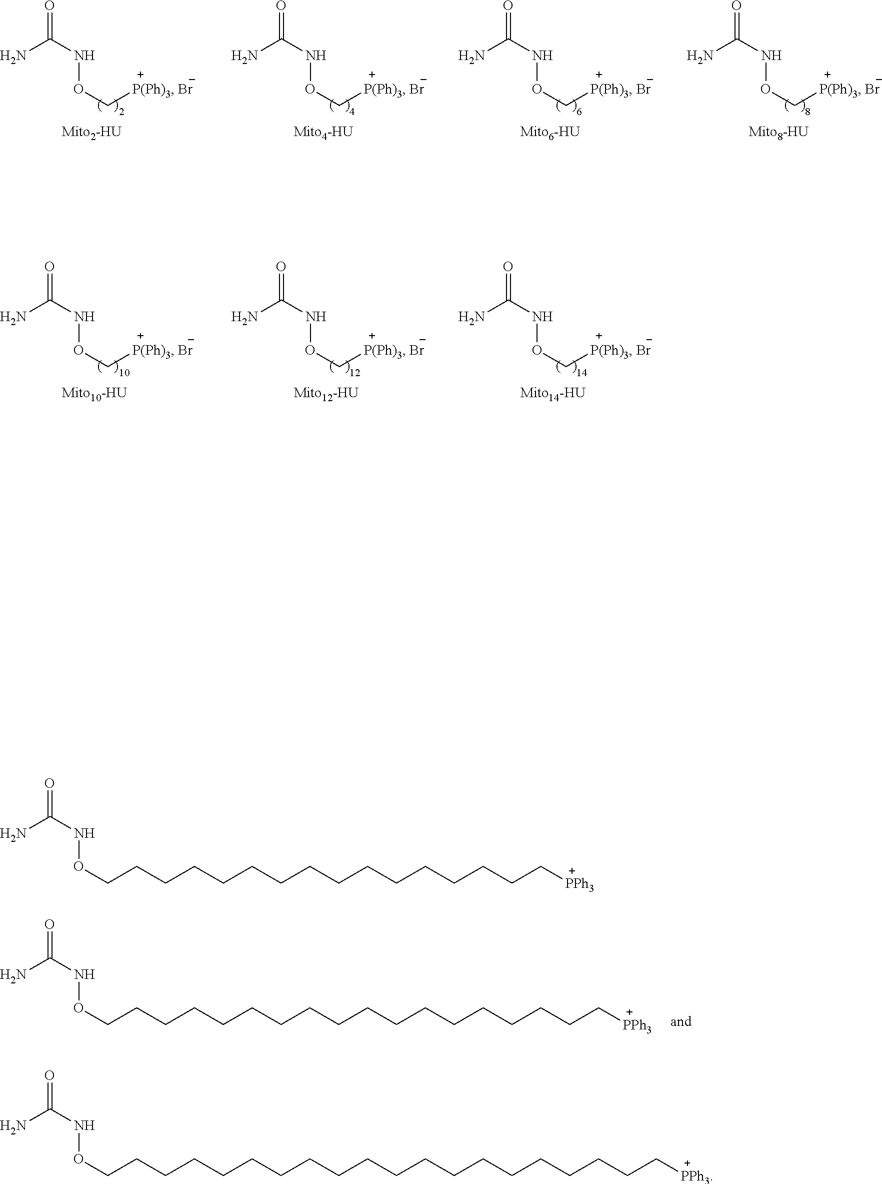

### Chemical Entities

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R²)—, —R¹C(O)N(R²)R³—, —C(O)N R² R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

### Pharmaceutical Compositions

In some embodiments, pharmaceutical compositions are provided herein. In some embodiments, the pharmaceutical compositions comprise mito-HU compounds with a formula selected from:

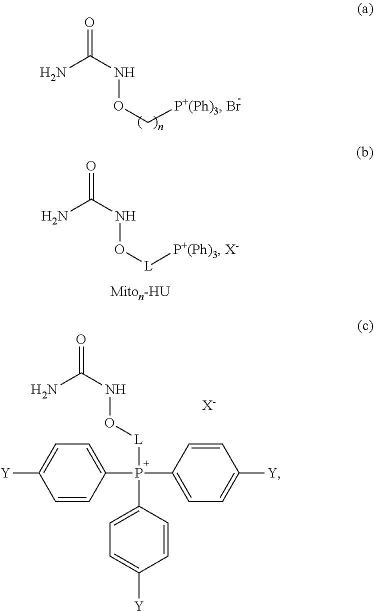

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
   X⁻ is selected from halogen, 2,2,2-trifluoroacetic acid (TFA), HO⁻, RCOO⁻, and acetic acid; and a pharmaceutically acceptable carrier or excipient.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a mito-HU compound may be administered as a single compound or in combination with another mito-HU compound.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder, e.g., a cell proliferative disorder such as cancer. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder, e.g., a cell proliferative disease or disorder including cancer.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods of Treatment

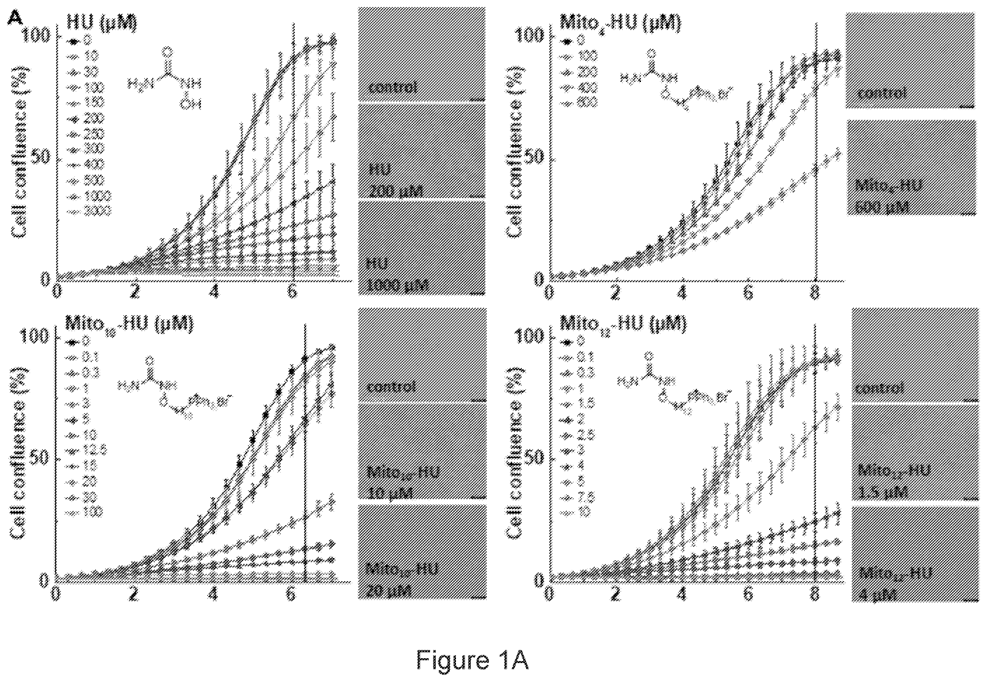

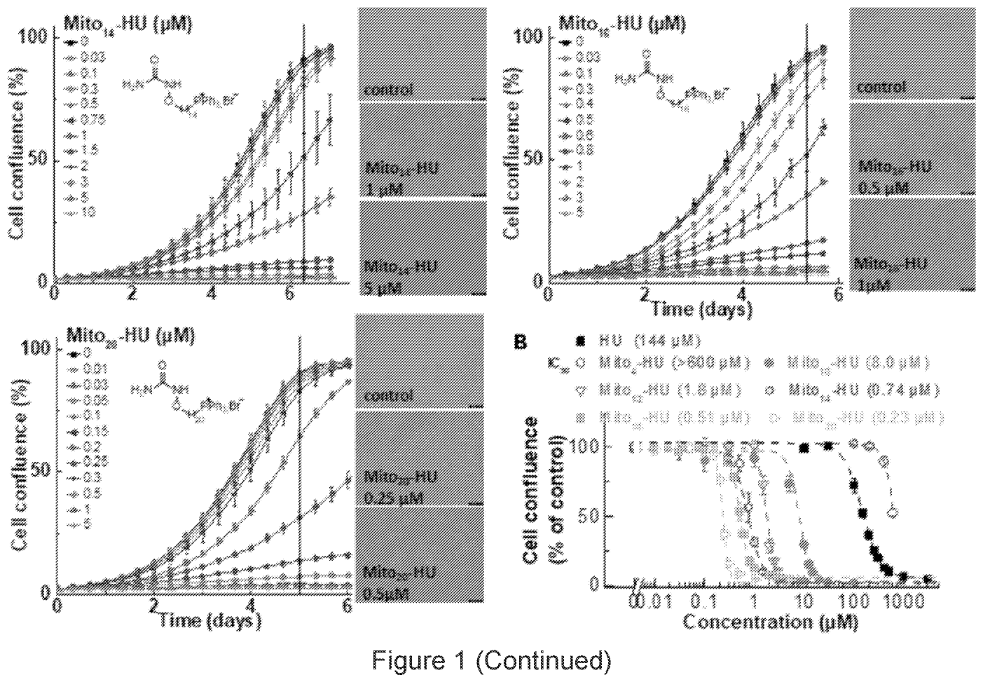

The Inventors demonstrated that mito-HU effectively inhibits the proliferation of cancer cells (FIG. 1). Therefore, in one aspect of the current disclosure, methods of treating cancer are provided. In some embodiments, methods of treating cancer in a subject in need thereof comprise administering a therapeutically effective amount of a mito-HU compound, wherein the mito-HU compound comprises a compound with a formula selected from:

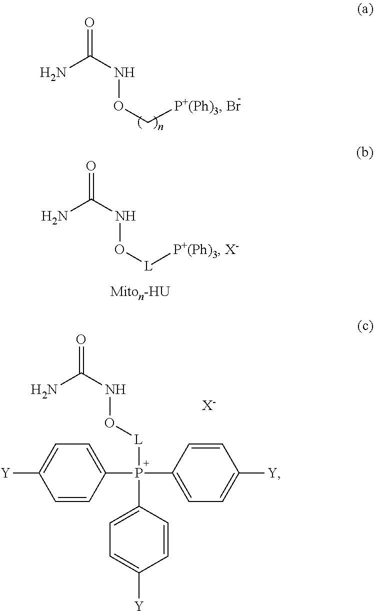

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid, to the subject to treat the cancer. In some embodiments, the compound is formulated into a pharmaceutical composition and administered to the subject.

A subject in need thereof, thus, comprises, in some embodiments of the methods of treatment of cancer, a subject suffering from, or diagnosed with, a cell proliferative disorder, e.g., cancer. The inventors tested the novel mito-HU compounds on pancreatic and colon cancer cells in vitro. However, without being bound by any theory or mechanism, the inventors believe that the mito-HU compounds inhibit critical metabolic pathways that are known to be upregulated in cancerous or transformed cells. Therefore, the inhibition of cancer cell proliferation demonstrated by the mito-HU compounds will likely have broad applicability to other cancer types.

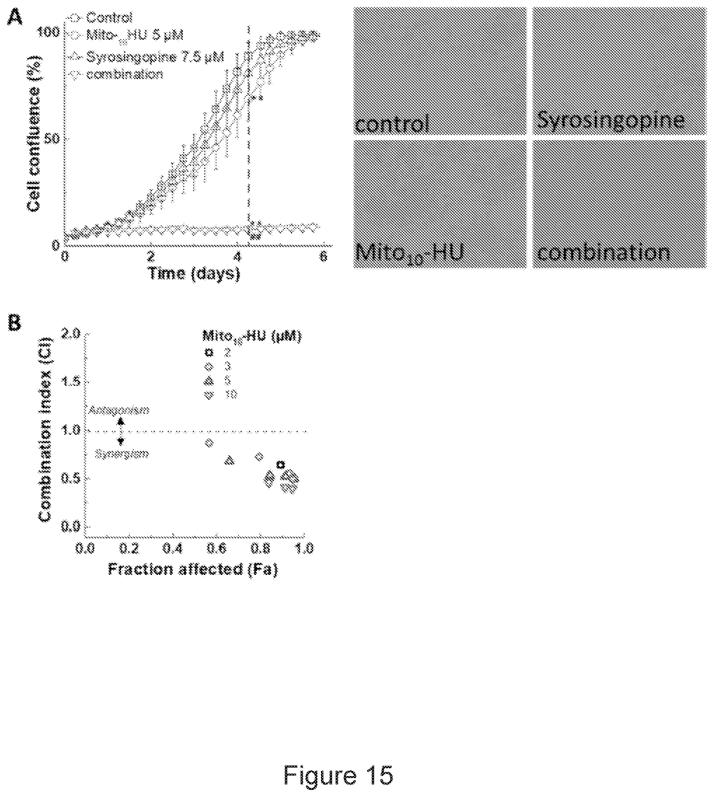

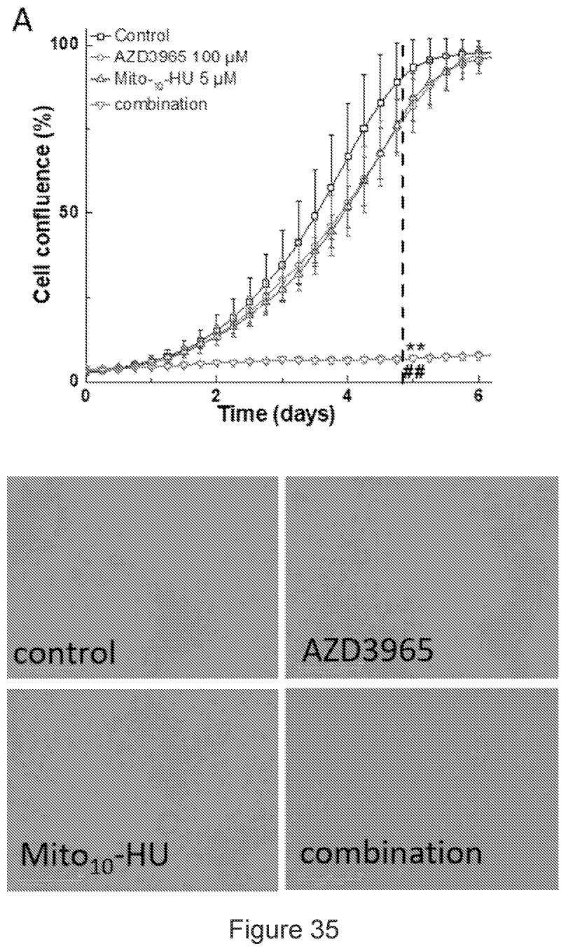

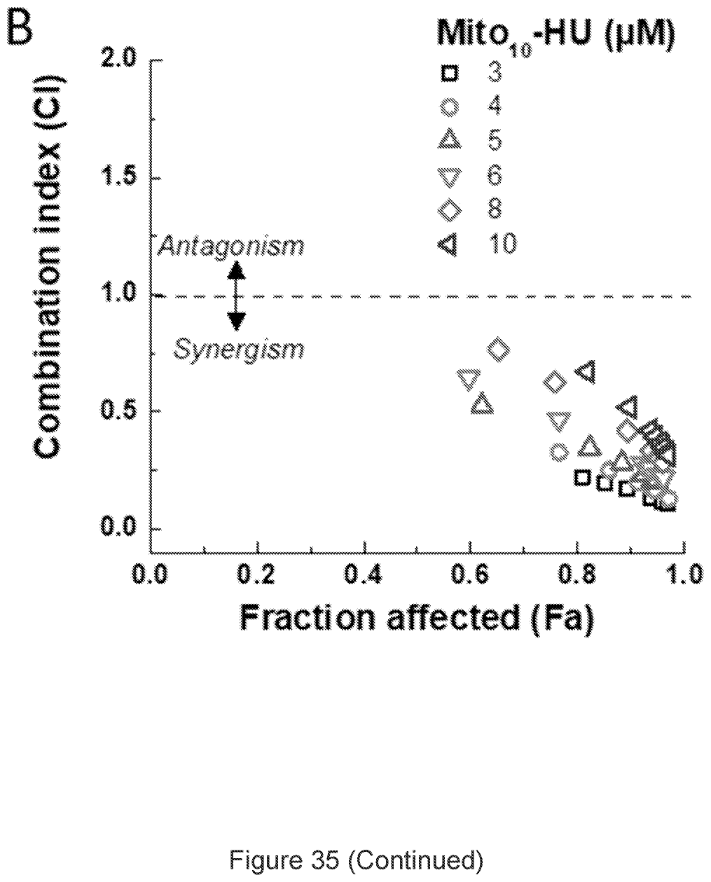

In addition, the inventors have discovered that the mito-HU compounds of the current disclosure act synergistically with inhibitors of the monocarboxylate 1 transporter (MCT-1) (FIGS. 15 and 35). MCT-1 inhibitors, e.g., AZD-3965, syrosingopine, AR-C 141990, AR-C155858, CHC, SR 13800 UK 5099, BAY 8002, 4-Chloro-α-cyanocinnamic acid (α-CHCA), and 7ACC2, inhibit the ability of cancer cells to transport lactate, and therefore, affect the cellular respiration of said cancer cells. Surprisingly, the inventors discovered that a sub-effective amount of MCT-1 inhibitor combined with mito-HU shows dramatic inhibition of cancer cell proliferation. In other words, a subtherapeutic amount of an MCT-1 inhibitor combined with a mito-HU compound act in concert to reduce cancer cell proliferation. Thus, the combination treatment using MCT-1 inhibitor and mito-HU may reduce side effects from the MCT-1 inhibitor because a lower dose is required for treatment, when combined with mito-HU. Therefore, in some embodiments, a subject is administered a therapeutically effective amount of an MCT-1 inhibitor and a mito-HU compound in order to treat a cell proliferative disease or disorder, e.g., cancer. In some embodiments, the MCT-1 inhibitor is selected from AZD-3965, syrosingopine, AR-C 141990, AR-C155858, CHC, SR 13800 UK 5099, BAY 8002, 4-Chloro-α-cyanocinnamic acid (α-CHCA), and 7ACC2. In some embodiments, the MCT-1 inhibitor is selected from AZD-3965 and syrosingopine. In some embodiments, the MCT-1 inhibitor and the mito-HU compound are administered at substantially the same time. As used herein, “substantially the same time” refers to, for example, the two compounds being administered or taken together as a single formulation, injection, pill, etc., or combination of formulations, injections or pills, etc. In other embodiments, the MCT-1 inhibitor and mito-HU compound may be administered at different times, e.g., the MCT-1 inhibitor is administered first and the mito-HU compound is administered second, or vice versa.

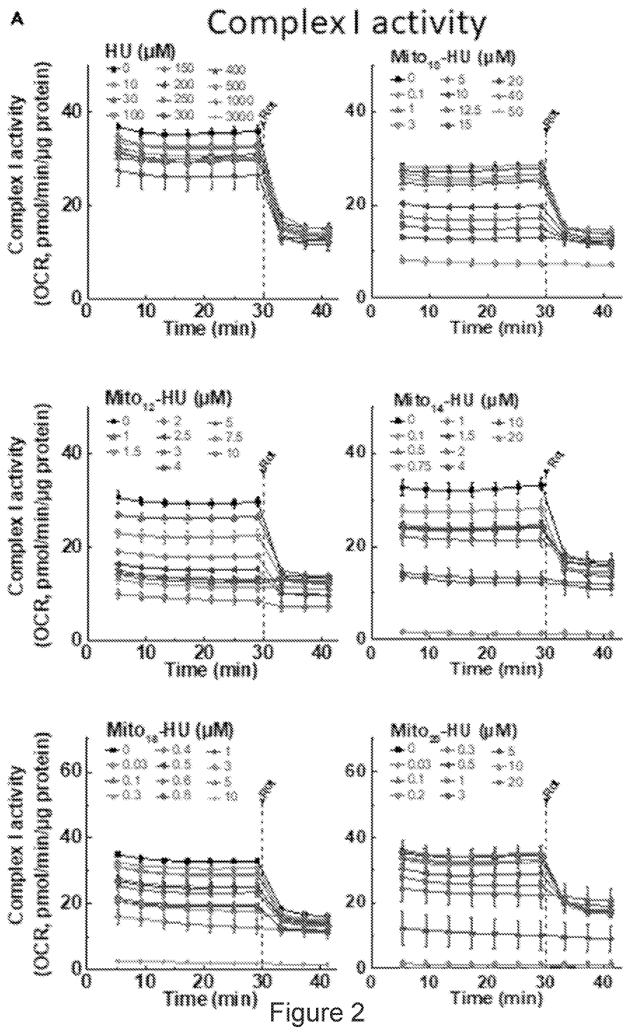

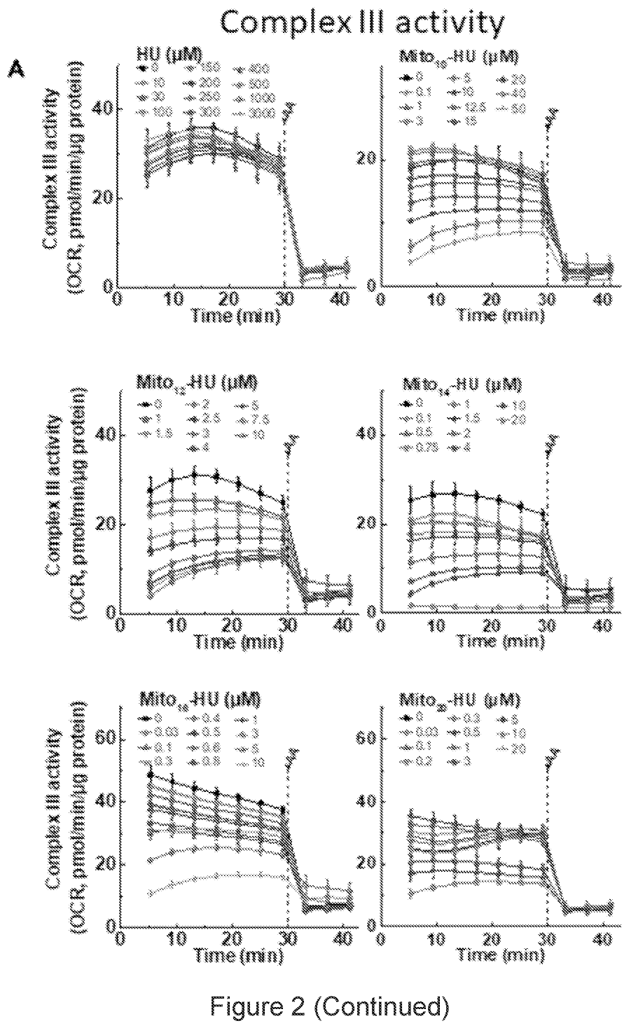

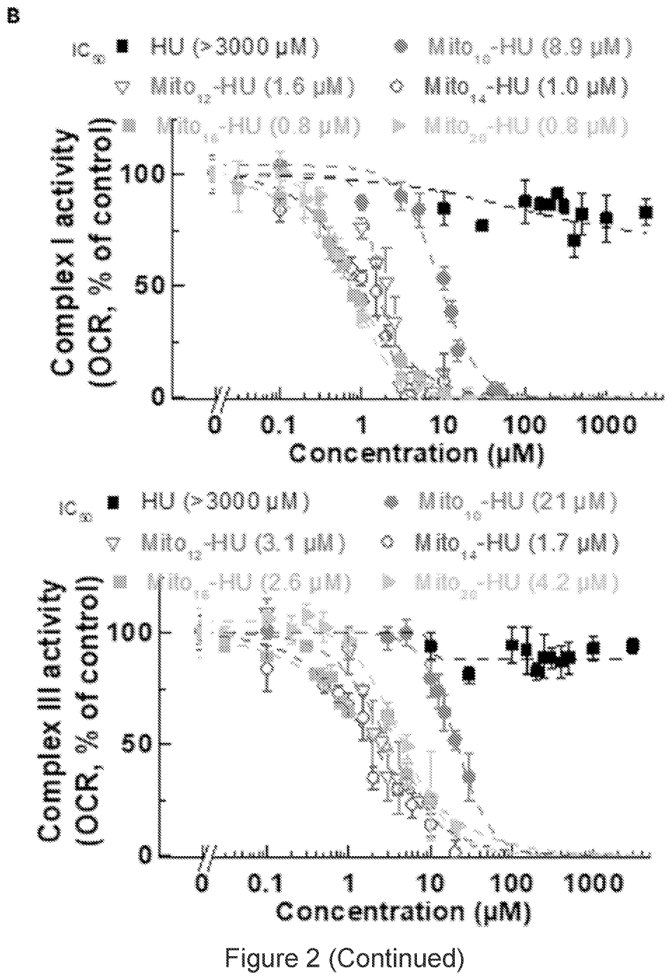

The drug metformin, a mainstay for the treatment of type II diabetes, has been shown to inhibit mitochondrial complex I. It is thought metformin's weak, but specific, inhibition of complex I contributes to the action of the drug in lowering blood glucose. See, for example, Vial et al. “Role of Mitochondria in the Mechanism(s) of Action of Metformin”, Front Endocrinol (Lausanne). 2019 May 7;10:294, which is incorporated by reference herein in its entirety. The inventors demonstrate herein that the novel mito-HU compounds inhibit mitochondrial complex I and III function (FIG. 2). Therefore, in another aspect of the current disclosure, methods of inhibiting mitochondrial complex I and complex III in a subject in need thereof are provided. A subject in need thereof, may, in some embodiments, comprise subjects with hyperglycemia, prediabetes, or diabetes mellitus type II. In some embodiments, the methods comprise administering a therapeutically effective amount of a compound with a formula selected from:

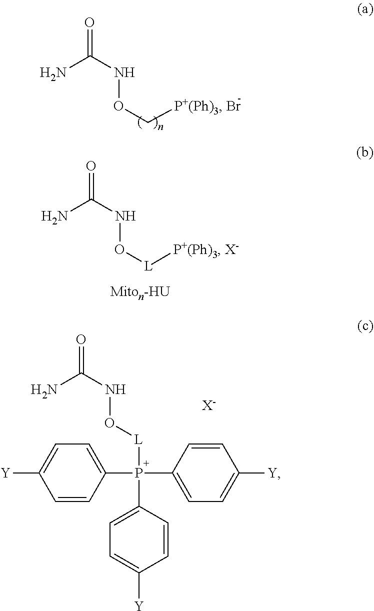

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid to the subject in order to inhibit, mitochondrial complex I, III or both in a subject. In some embodiments, the compound is formulated into a pharmaceutical composition and administered to the subject.

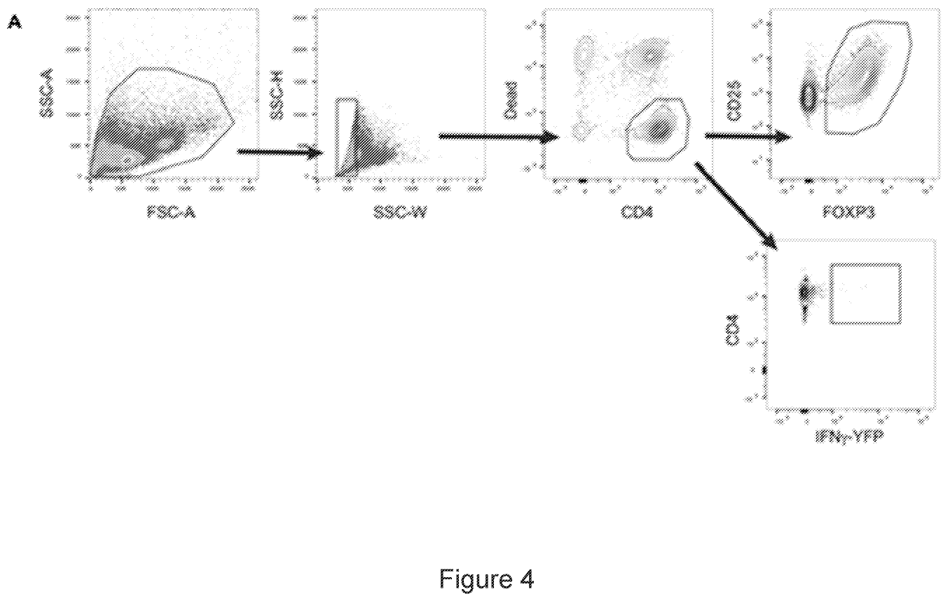

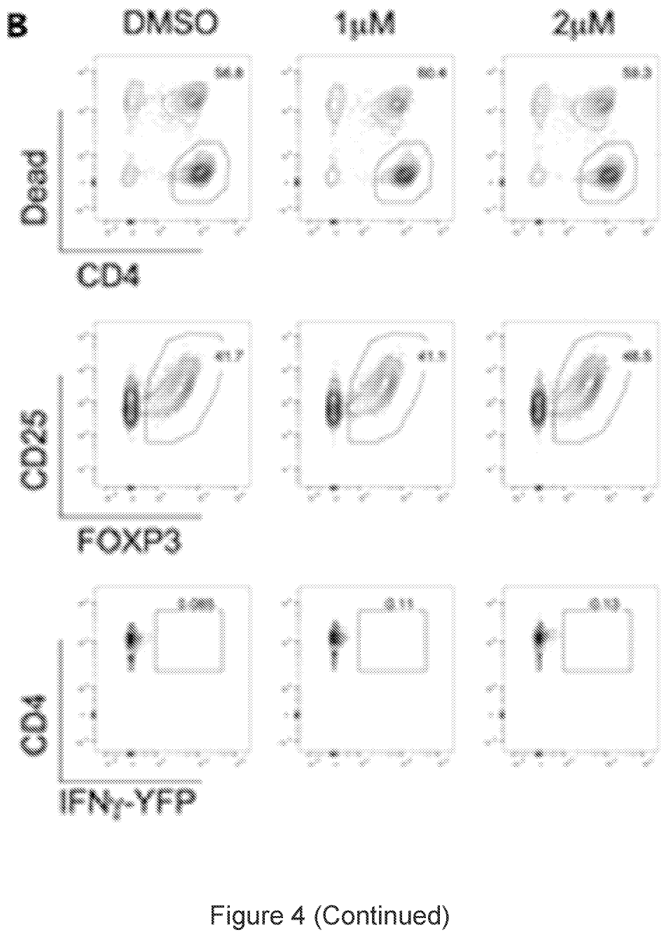

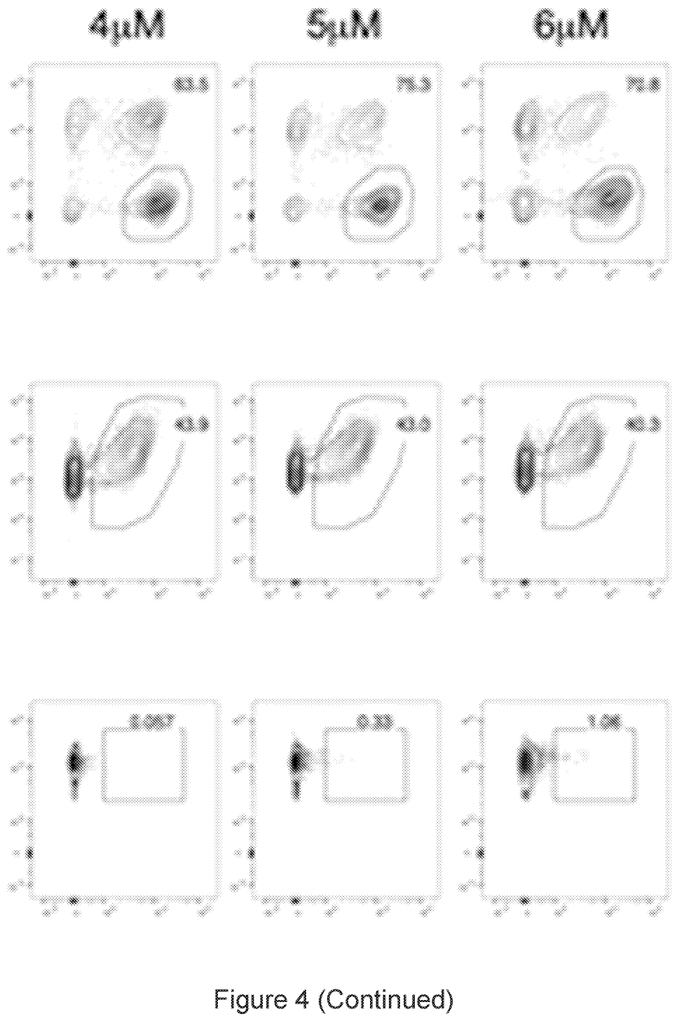

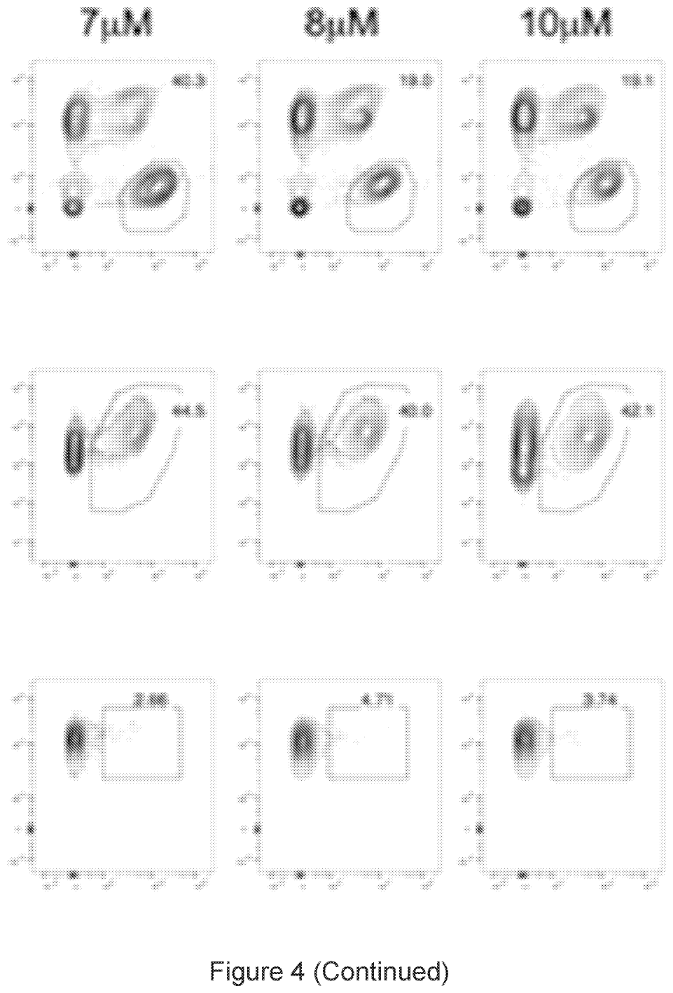

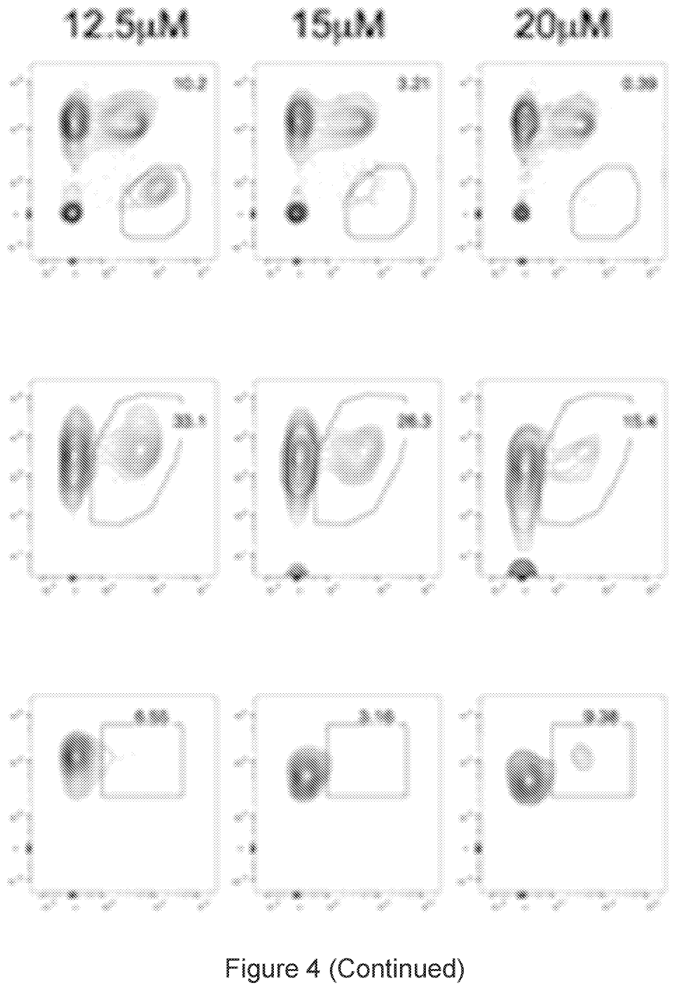

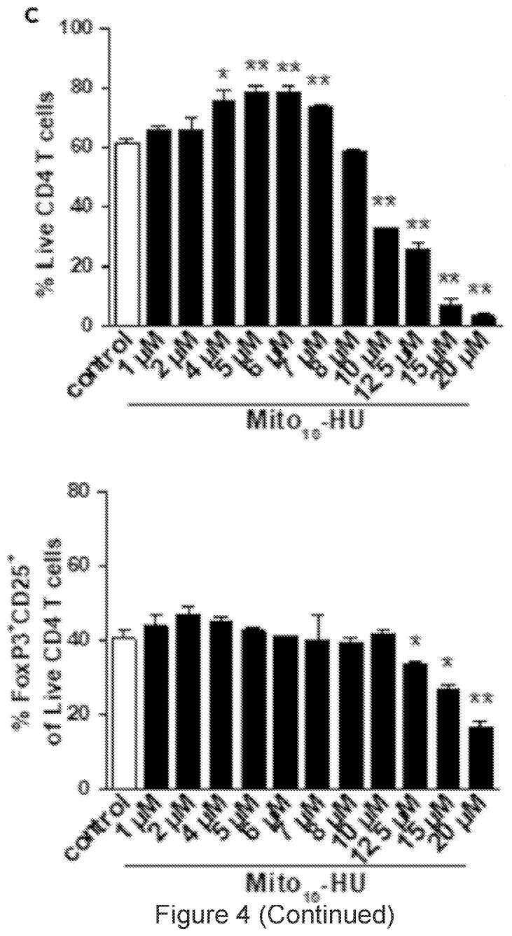

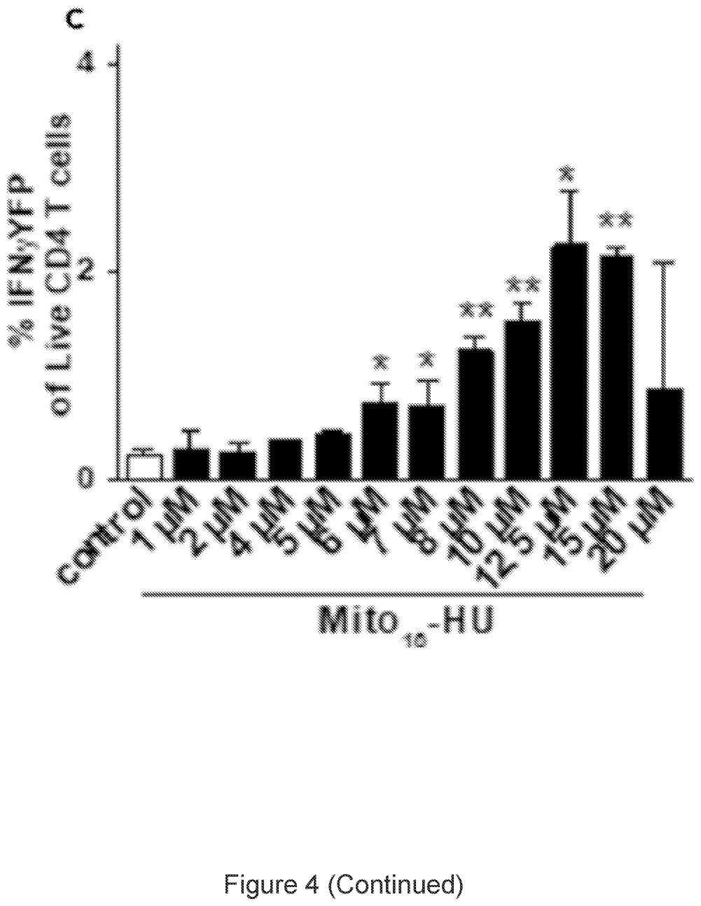

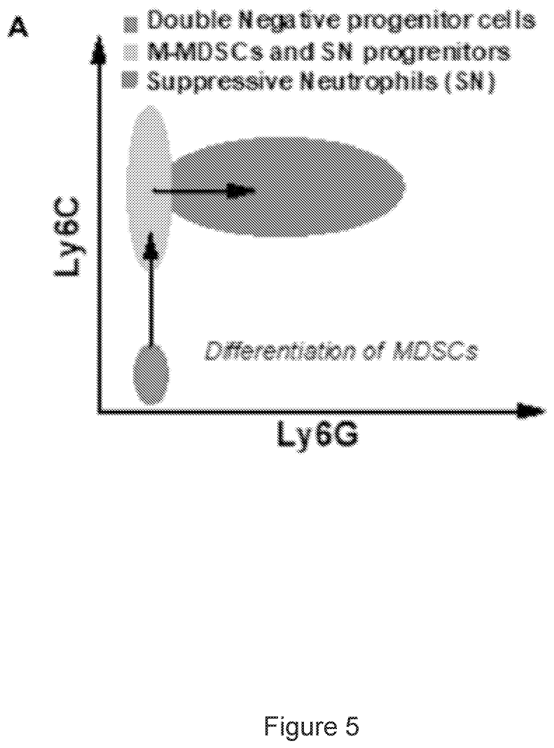

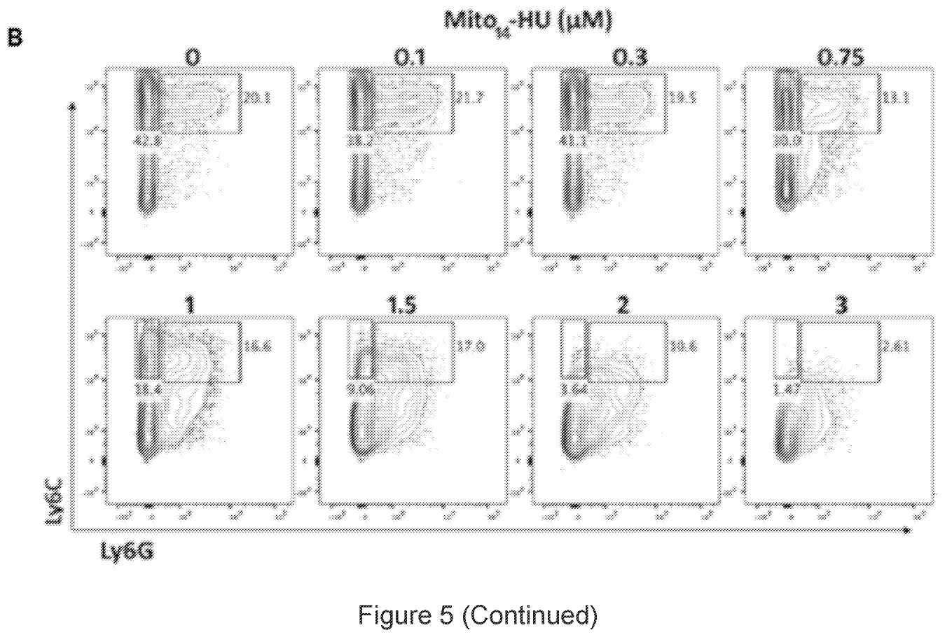

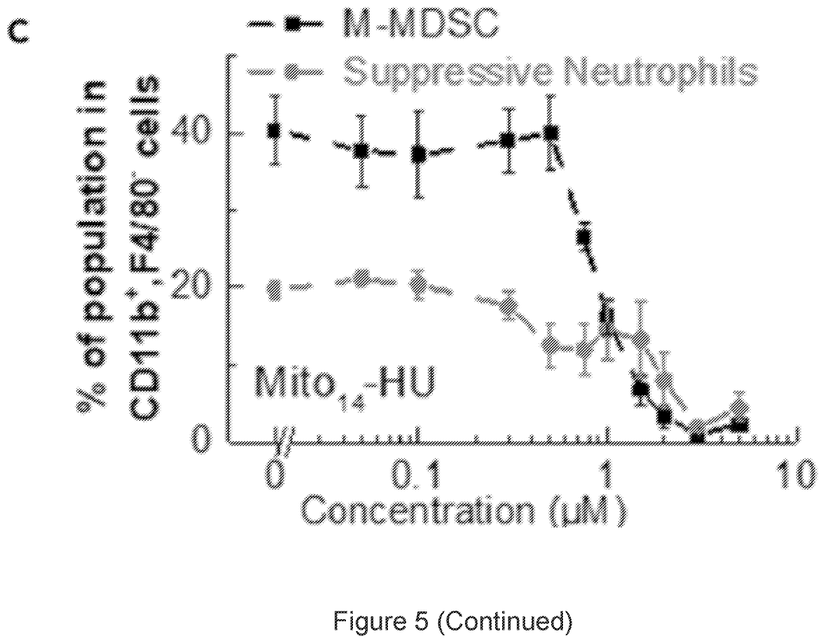

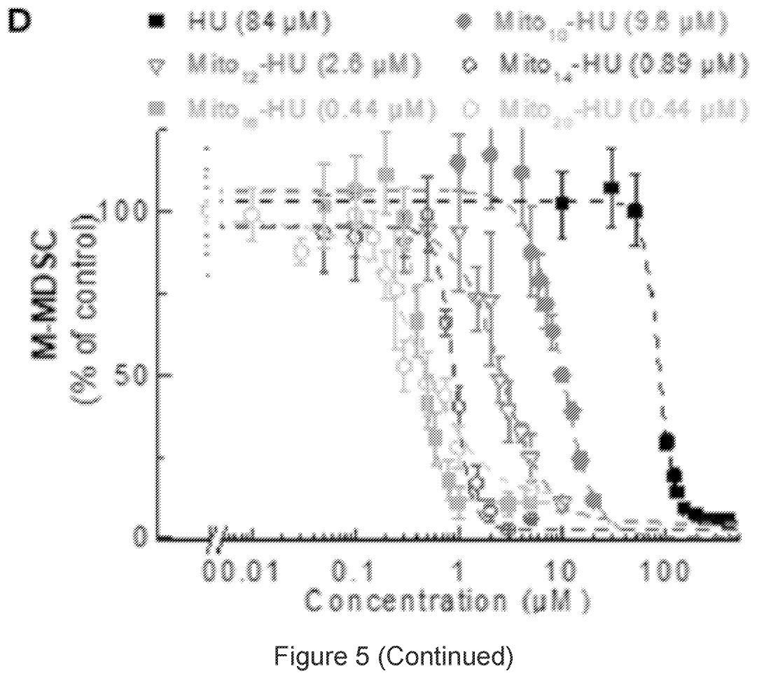

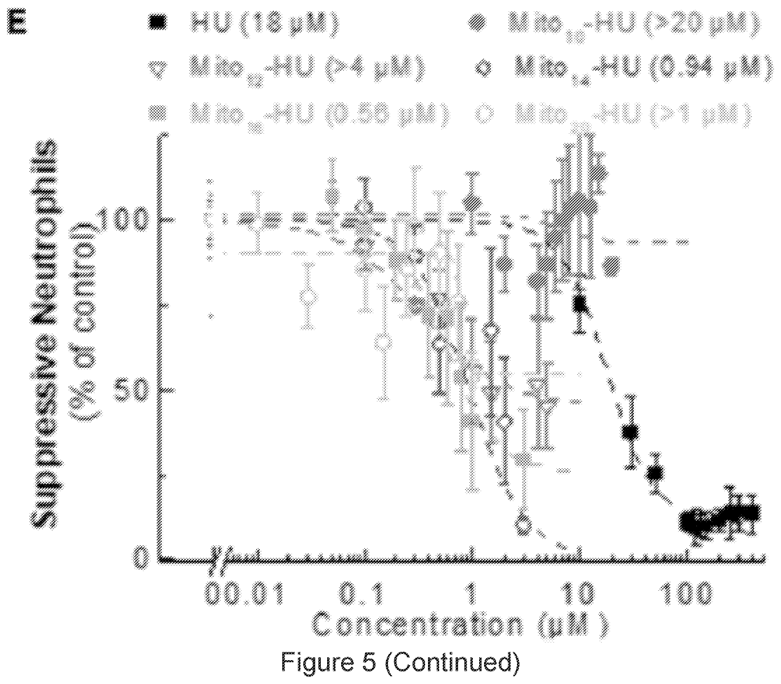

The Inventors demonstrate herein that the novel mito-HU compounds have the ability to increase IFN-γ production by CD4 T cells while reducing the frequency of immunosuppressive FoxP3⁺ T_{regs} (FIG. 4) and reduce myeloid derived suppressor cell frequency (FIG. 5) in vitro. Thus, the novel mito-HU compounds reduce two key immunosuppressive cell populations that are present in the tumor microenvironment (TME) and further increase the expression of the inflammatory cytokine interferon-γ (IFN-γ). Therefore, in another aspect of the current disclosure, methods of increasing an immune response to cancer cells in a subject in need thereof are provided. In some embodiments, the methods comprise administering a therapeutically effective amount of a compound with a formula selected from

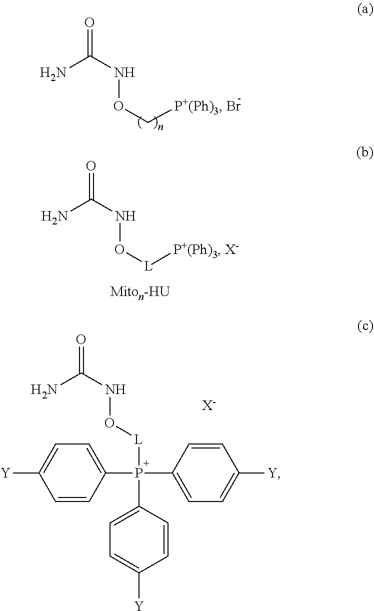

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid to the subject in order to increase an immune response to the cancer cells. In some embodiments, the compound is formulated into a pharmaceutical composition and administered to the subject.

In one embodiment, compound (c) is

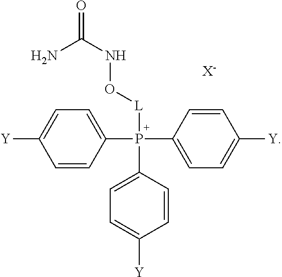

A discussed above, the novel mito-HU compounds of the instant disclosure have the ability to increase IFN-γ production by CD4 T cells while reducing the frequency of immunosuppressive FoxP3⁺ T_{regs} (FIG. 4) and reduce myeloid derived suppressor cell frequency (FIG. 5) in vitro. Thus, the novel mito-HU compounds reduce two key immunosuppressive cell populations and further increase the expression of the inflammatory cytokine IFN-γ in CD4 T cells in vitro. Therefore, in another aspect of the current disclosure, methods of reducing the number of immunosuppressive cells in a subject in need thereof are provided. In some embodiments, the method comprises administering a therapeutically effective amount of a compound with a formula selected from

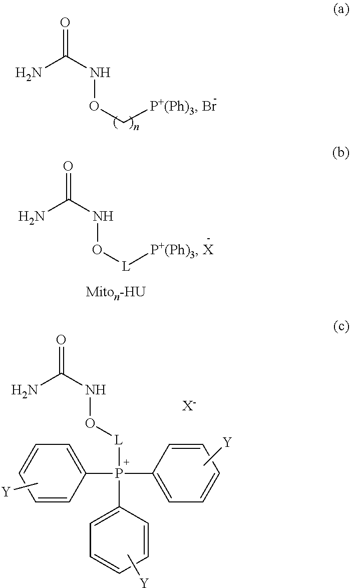

preferably in one embodiment, the compounds are selected from:

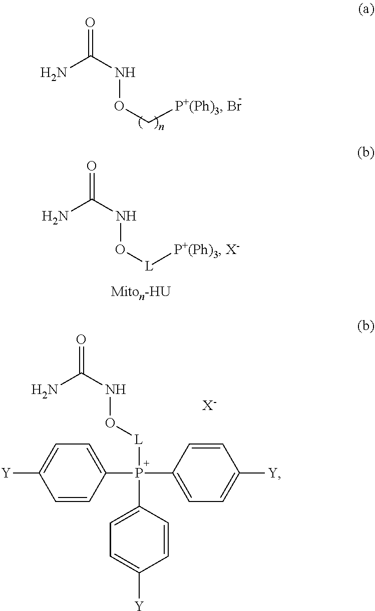

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid to the subject in order to reduce the number of immunosuppressive cells in the subject. In some embodiments, the compound is formulated into a pharmaceutical composition and administered to the subject.

HU has been used in combination with radiation therapy as a “radiation sensitizer” or a compound that “potentiates radiation therapy”, which are used interchangeably. As used herein, “radiation sensitizer” or a compound that “potentiates radiation therapy” refer to compounds or agents that enhance the cell killing from radiation of tumor cells. Mito-HU and related analogs have enhanced potency to inhibit cancer cell respiration, thus, enhancing the oxygen levels in hypoxic regions and enhancing radiation sensitization or potentiation. Therefore, in another aspect of the current disclosure, methods of potentiating radiation therapy in a subject in need thereof are provided. In some embodiments, the methods comprise administering a compound with a formula selected from

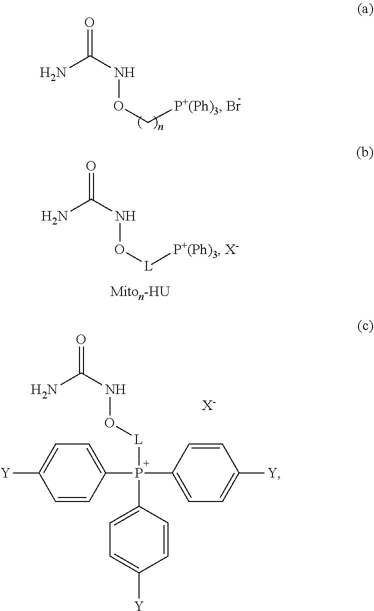

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid to a subject to potentiate radiation therapy and treating the subject with radiation therapy.

### General Methods of Use of Mito-HU Compounds

The inventors demonstrated the novel mito-HU compounds potently inhibit the proliferation of cancer cells (FIG. 1). Therefore, in another aspect of the current disclosure, methods of inhibiting proliferation of a cancer cell are provided. In some embodiments, the method a comprise contacting a cancer cell with an effective amount of a compound with a formula selected from

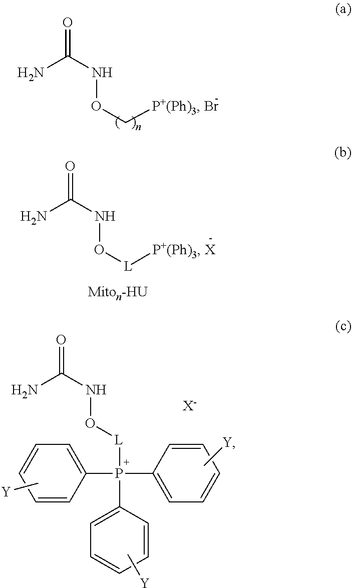

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
   each Y is independently selected from —H, —CF₃, methyl (Me), Cl, OMe, C(O)CH₃, NO₂, N(Me)₂;
   and
   X⁻ is selected from halogen, 2,2,2-trifluoroacetic acid (TFA), HO⁻, RCOO⁻, and acetic acid. In some embodiments, an effective amount of the compound is a concentration of less than about 20 μM. In some embodiments, an effective amount of the compound is a concentration of less than about 10 μM. In some embodiments, an effective amount is a concentration of 1 nM, 10 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, or 20 μM.

In one embodiment, the compound of (c) is

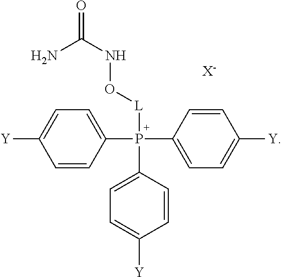

The inventors discovered that the mito-HU compounds increase the frequency of interferon-γ (IFN-γ) in CD4 T cells in vitro. Therefore, in another aspect of the current disclosure, methods of increasing the production of IFN-γ in a T cell are provided. In some embodiments, the methods comprise contacting the cell with an effective amount of a compound with a formula selected from

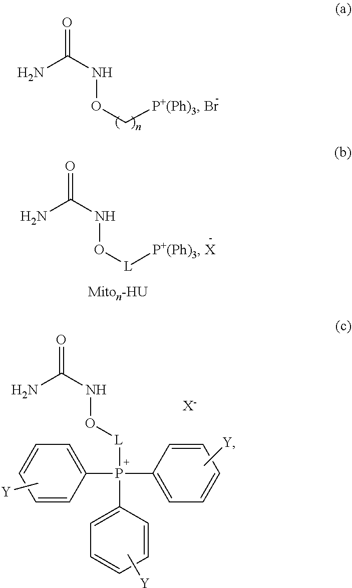

wherein n is an integer from 1-20, L is a linker, wherein the linker is selected from an unsubstituted C₁-C₂₀ alkyl, substituted C₁-C₂₀ alkyl, C₂-C₂₀ alkenyl, phenyl, phenyl substituted C₁-C₂₀ alkyl, cycloalkyl substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid. In some embodiments, an effective amount of the compound is a concentration of less than about 20 μM. In some embodiments, an effective amount of the compound is a concentration of less than about 10 μM. In some embodiments, an effective amount is a concentration of 1 nM, 10 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, or 20 μM. In some embodiments, the T cell is a CD4 T cell. In some embodiments, the T cell is a CD8 T cell.

In one embodiment, compound (c) is

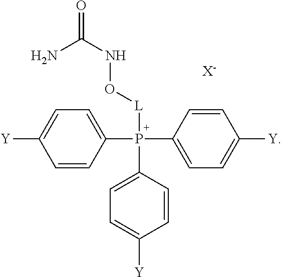

### EXAMPLES

The following examples are shown to further illustrate the disclosed invention and are not intended to limit the disclosed invention, as recited in the claims.

### Example 1: Mitochondria-Targeted Hydroxyurea Inhibits OXPHOS and Induces Antiproliferative and Immunomodulatory Effects

Hydroxyurea (HU), also known as hydroxycarbamide (trade names Hydrea and Droxia), is a chemotherapeutic agent used to treat melanoma, refractory chronic myelocytic leukemia (CML), recurring and inoperable ovarian cancer, and squamous cell carcinoma of the head and neck (Spivak and Hasselbalch, 2011; Madaan et al., 2012; Singh and Xu, 2016; Grund et al., 1977). HU belongs to a group of chemotherapeutic agents known as “antimetabolites” that interfere with the production of nucleic acids (Koc, et al., 2004; Singh and Xu, 2016). HU inhibits the multi-enzyme complex, ribonucleotide diphosphate reductase (RDR), an enzyme that catalyzes the conversion of ribonucleotide to deoxyribonucleotide during de novo DNA synthesis (Teng et al., 2018; Koc, et al., 2004; Zhou et al., 2013). HU has been used in combination with other modalities, conventional chemotherapeutics, and radiation therapy. RDR also is involved in DNA repair. When combined with radiation, the therapeutic efficacy of HU is increased because it inhibits DNA repair (Teng et al., 2018; Madaan et al., 2012; Singh and Xu, 2016). In cancer patients, HU has been used extensively as a radiation sensitizer by synchronizing cells in a radiation-sensitive S-phase of the cell cycle (Yarbro, 1992). In addition, HU inhibits the repair of radiation-induced DNA damage (Singh and Xu, 2016). HU is blood-brain barrier permeable and, in combination with temozolomide, was used as an adjuvant therapy for glioblastoma patients (Teng et al., 2018).

Several decades ago, HU was shown to inhibit L1210 leukemia cells and various solid tumors (Mai et al., 2010; Ren et al., 2002). Later, HU was shown to be effective against myeloproliferative disorders, CML, and polycythemia rubra vera (Spivak and Hasselbalch, 2011). It was also postulated that HU could stimulate an immune response in melanoma and lung cancer by recruiting components of the innate immune system (Cheng et al., 2020b; Oo et al., 2019). Typically, high concentrations of HU are required for in vitro and in vivo efficacy in chemotherapy (Singh and Xu, 2016). The high dose of HU is attributed to its side effects and drug resistance (Madaan et al., 2012; Singh and Xu, 2016). HU (H₂N—CO—NHOH) is the hydroxylated analog of urea (NH₂—CO—NH₂). The hydroxyl group (⁻OH in HU) is thought to be critical for its antiproliferative and antitumor mechanisms (Madaan et al., 2012; Singh and Xu, 2016). In this study, we modified the structure of HU by replacing the ⁻OH group with a triphenylphosphonium cation (TPP⁺)-containing group. These modified HU derivatives inhibited mitochondrial oxygen consumption more potently than HU and are, therefore, designated as mitochondria-targeted HUs (Mito-HUs).

Increasing evidence suggests that certain subtypes of cancer cells, including cancer stem cells and chemotherapy-resistant cancer cells, utilize oxidative phosphorylation (OXPHOS) to obtain the energy needed for survival and proliferation (Fiorillo et al., 2016; Xu et al., 2020). Targeting OXPHOS, especially mitochondrial complex I, is emerging as a potential antitumor strategy to treat several types of cancer (Cheng et al., 2013, 2016, 2019; Boyle et al., 2018; Weinberg and Chandel, 2015; Xu et al., 2020; Fischer et al., 2019; Pan et al., 2018). As previously suggested (American Association for Cancer Research, 2019), OXPHOS inhibitors that target cancer cells will also target cancer-associated immune cells. Reports suggest that suppression of OXPHOS function in cancer cells could influence the tumor microenvironment (TME) by inhibiting hypoxia and enhancing the antitumor response (Xu et al., 2020). However, to our knowledge, mitochondria-targeted derivatives with varying hydrophobicities on cytotoxic and tumor suppressive cells in the TME have not been thoroughly tested. Reports indicate that cancer patients with enhanced myeloid-derived suppressor cells (MDSCs) and regulatory T cells (T_{regs}) have lower survival compared to patients with decreased MDSCs (Solito et al., 2011; Saleh and Elkord, 2020). Therefore, MDSCs and T_{regs} are promising targets for antitumor treatment (Kawano et al., 2015).

The chemotherapeutic agents (gemcitabine and 5-fluorouracil) currently used to inhibit MDSCs cause bone marrow suppression; clearly, less toxic and targeted agents are needed to inhibit MDSCs and/or suppressive neutrophils as well as to enhance the cytotoxic antitumor function of T cells (Sawant et al., 2013; Law et al., 2020; Hossain et al., 2015). MDSCs suppress T cells that destroy tumor cells (Nagaraj et al., 2013). Broadly designed MDSCs comprise both monocytic MDSCs (M-MDSC) and suppressive neutrophils. In this study, we investigated the antiproliferative and immunomodulatory effects of Mito-HUs with varying hydrophobicities. The results show that more hydrophobic Mito-HUs potently inhibit the proliferation of tumor cells, inhibit the differentiation of M-MDSCs and suppressor neutrophils, and stimulate the effector T cell (T_{eff}) response.

### Results

Substitution of the ⁻OH group in HU with increasing alkyl side chain lengths increases the hydrophobicity in Mito-HUs

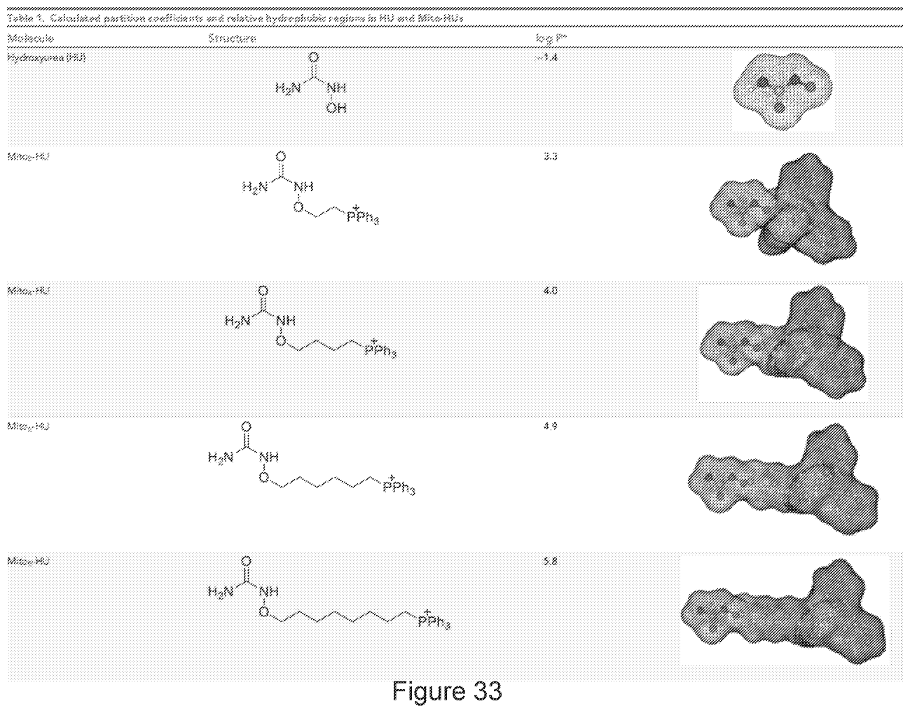

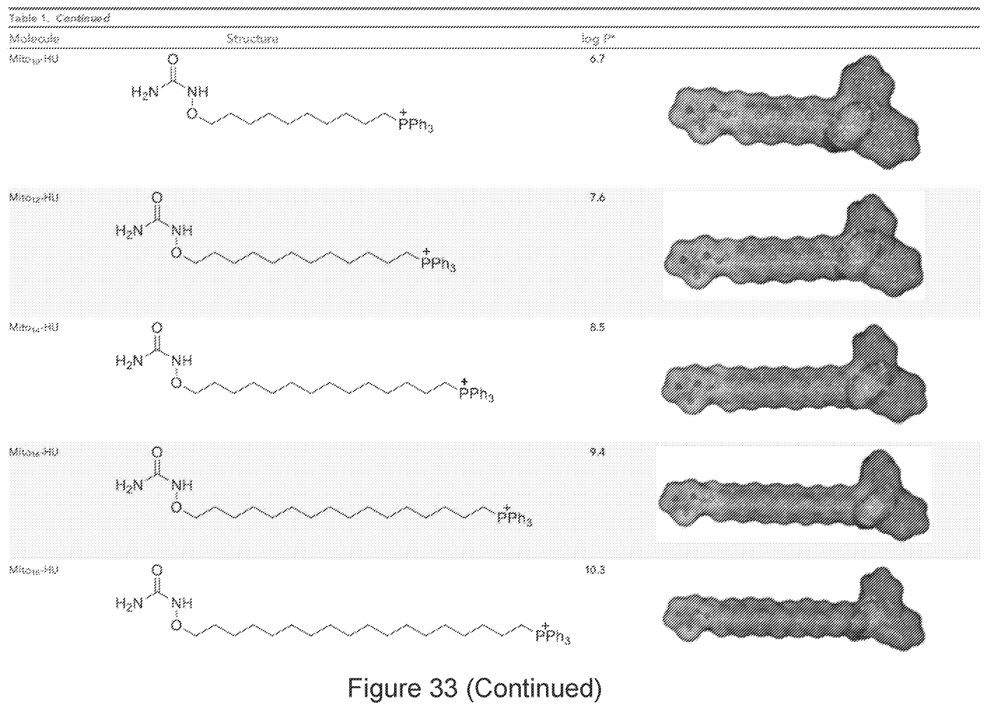

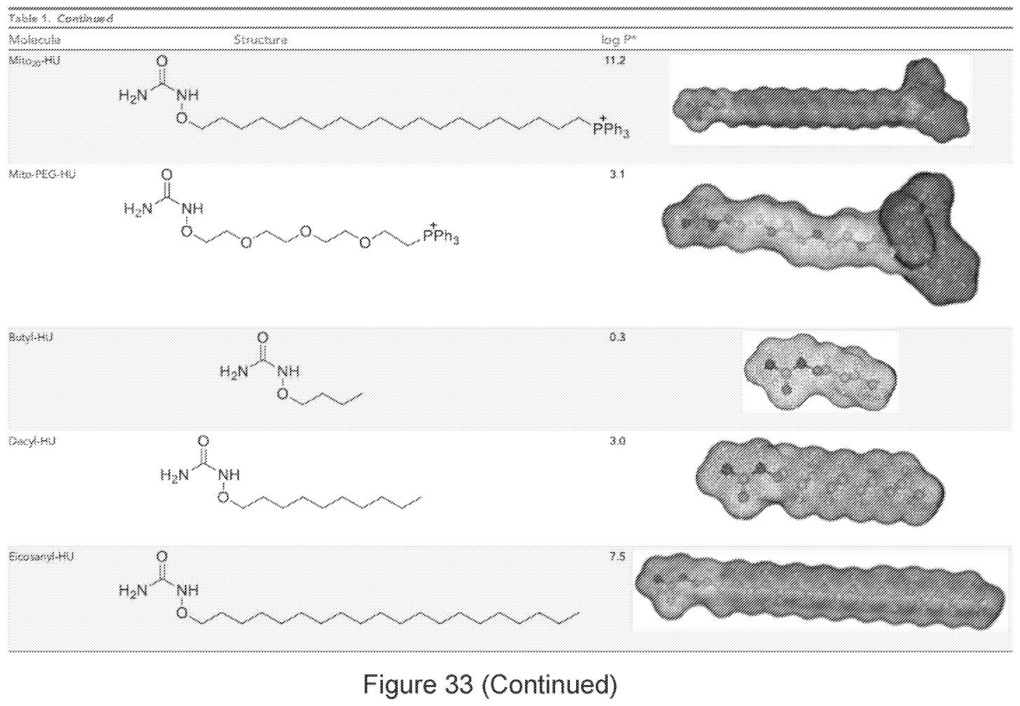

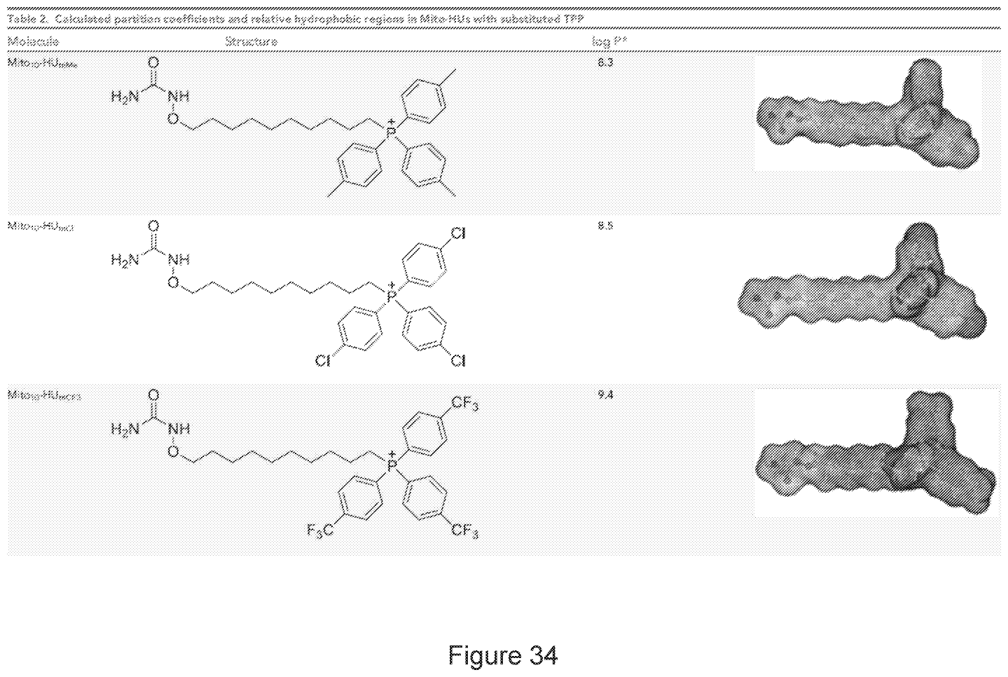

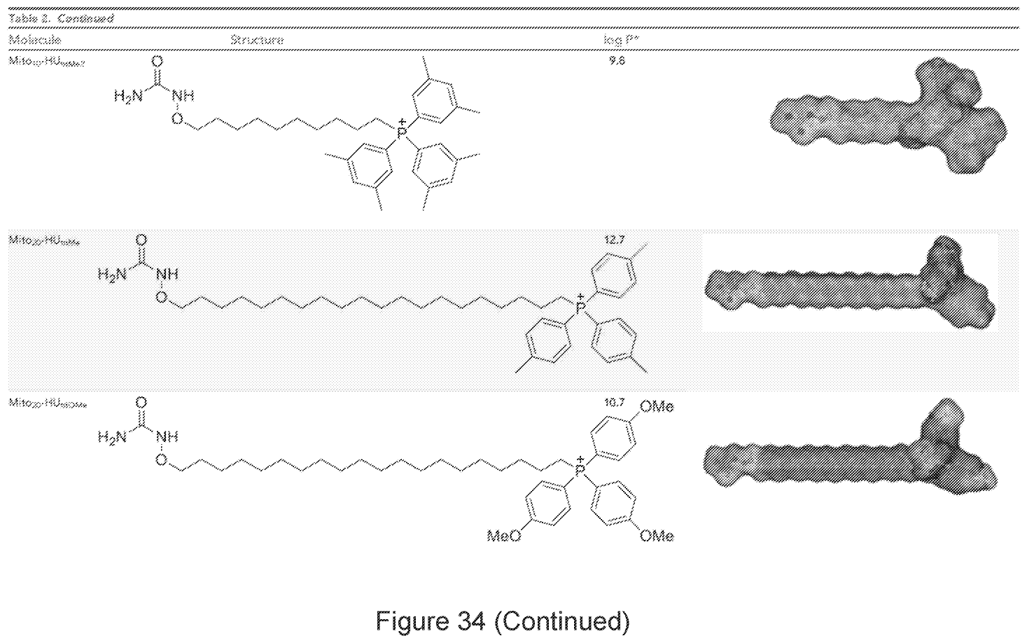

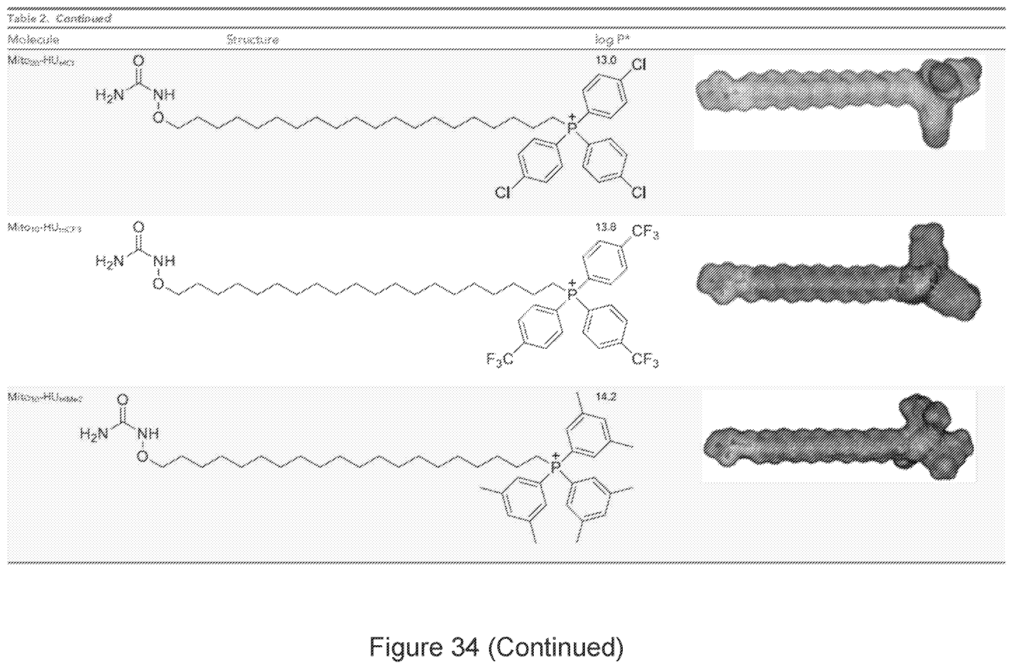

To assess the relative hydrophobicity of HU and Mito-HU derivatives, we calculated the octanol/water partition coefficients (log P) using a QSAR (quantitative structure-activity relationships) analysis and rational drug design as a measure of molecular hydrophobicity (FIG. 33). This method also uses a consensus model built using the ChemAxon software (San Diego, CA) (Viswanadhan et al., 1989; Klopman et al., 1994). FIG. 33 lists the log p values along with the calculated regions of the relative hydrophilic and hydrophobic regions. HU is very polar, and the calculated log p value is −1.4 (FIG. 33). The calculated value agrees with the experimentally determined oil-to-water log p value (i.e., −1.8) for HU (Ren et al., 2002). The extreme water solubility of HU, necessitating the use of a high dosage and frequency of administration for therapeutic efficacy, resulted in the rapid development of drug resistance (Madaan et al., 2012; Singh and Xu, 2016). Thus, we surmised that increasing its hydrophobicity might enhance drug potency. FIG. 33 presents the structures of various Mito-HUs and controls as well as the calculated hydrophobicity (see supplemental information). Results shown below suggest that derivatization of the ⁻OH group in HU with a TPP⁺-containing alkyl side chain enhances the antiproliferative efficacy, with increasing hydrophobicity.

Increased hydrophobicity associated with increased antiproliferative potency and decreased mitochondrial oxygen consumption

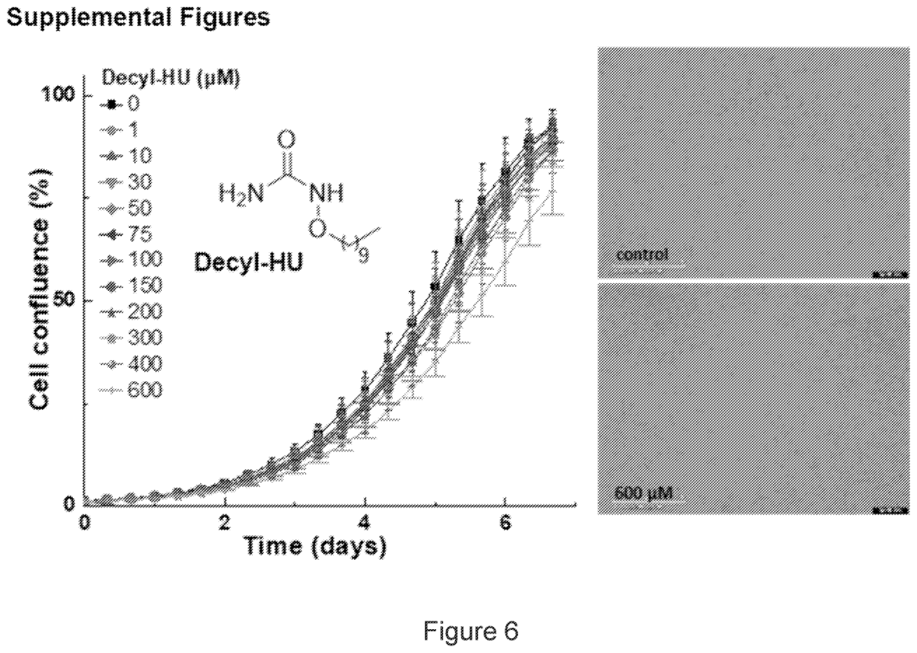

Cell proliferation was monitored continuously in an IncuCyte Live-Cell Analysis system (Cheng et al., 2013, 2016; Boyle et al., 2018) using HU and Mito-HUs (Mito₄-HU, Mito₁₀-HU, Mito₁₂-HU, Mito₁₄-HU, Mito₁₆-HU, and Mito₂₀-HU; FIG. 1A). Mito₄-HU, Mito₁₀-HU, Mito₁₂-HU, Mito₁₄-HU, Mito₁₆-HU, and Mito₂₀-HU dose-dependently inhibited proliferation of HCT116 human colon cancer cells (FIGS. 1A and 1B). A Mito-HU with an alkyl chain length R10 is more potent than HU at inhibiting the proliferation of HCT116 cells. FIG. 1B shows the cell confluence (indicated by a dotted line) as a function of Mito-HU concentration, and the half maximal inhibitory concentration (IC₅₀, mM) values of Mito₄-HU, Mito₁₀-HU, Mito₁₂-HU, Mito₁₄-HU, Mito₁₆-HU, and Mito₂₀-HU are >600 mM, 8.0 mM, 1.8 mM, 0.74 mM, 0.51 mM, and 0.23 mM, respectively (Table 2). As compared with Mito-HUs, HU inhibited cell proliferation at much higher concentrations (FIG. 1B). These results suggest that enhancing the hydrophobicity by attaching a longer aliphatic chain containing a TPP⁺ group to HU greatly increases the antiproliferative potency. The antiproliferative effects of Mito₁₂-HU, Mito₁₄-HU, Mito₁₆-HU, and Mito₂₀-HU are ˜100-600 times more potent than those of HU. In control experiments, we used compounds (decyl-HU) with an alkyl carbon-carbon side chain length similar to that of Mito₁₀-HU but lacking the TPP⁺. Decyl-HU (IC₅₀>600 mM; FIG. 6) was much less effective than the corresponding Mito₁₀-HU (IC₅₀=8.0 mM; FIG. 1B). Results obtained using decyl-HU confirm that the TPP⁺ moiety in Mito₁₀-HU is required to enhance its antiproliferative activity. The statistical analyses of results (FIG. 1) are shown in Table 3.

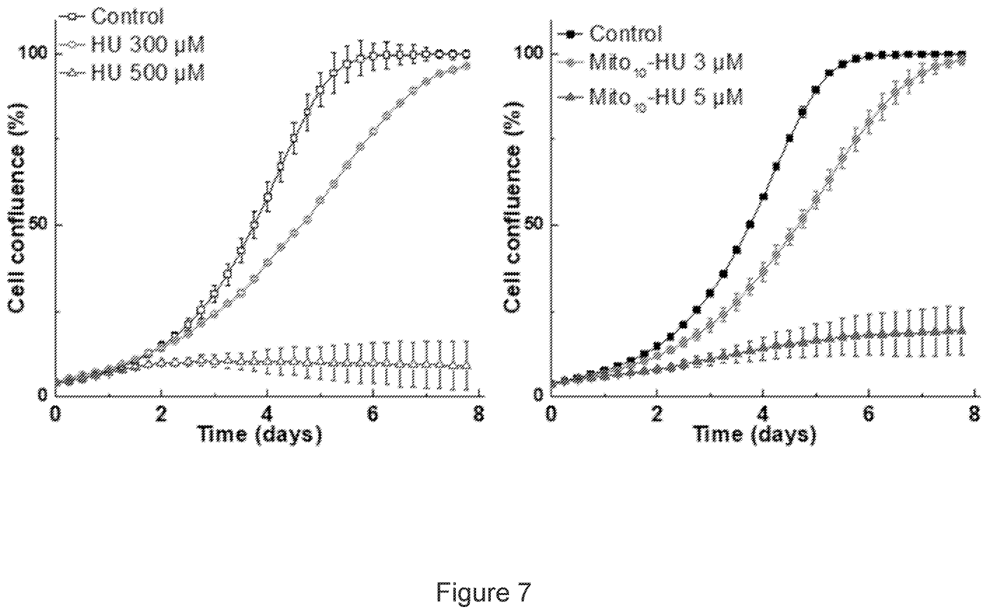

We also tested the relative inhibitory effects of HU and Mito₁₀-HU on the proliferation of MiaPaCa-2 human pancreatic cancer cells (FIG. 7). Mito₁₀-HU was nearly 100 times more potent than HU in inhibiting MiaPaCa-2 cell proliferation (FIG. 7).

Mito₁₀-HU_{NHTPP}⁺ (FIG. 8) was designed and synthesized to compare the effects of O-alkylation and N-substitution on HU by the aliphatic chain containing a TPP⁺. Results also show that attaching a 10-carbon aliphatic chain containing a TPP⁺ group to the amino group of HU (as in Mito₁₀-HU_{NHTPP}⁺) inhibited HCT116 cell proliferation (FIG. 8). This inhibition is similar to that of a 10-carbon aliphatic chain containing a TPP⁺ group to the ⁻OH group in HU (as in Mito₁₀-HU) (FIG. 8). This result confirms that the antiproliferative effect of Mito₁₀-HU is not dependent on the free amino group.

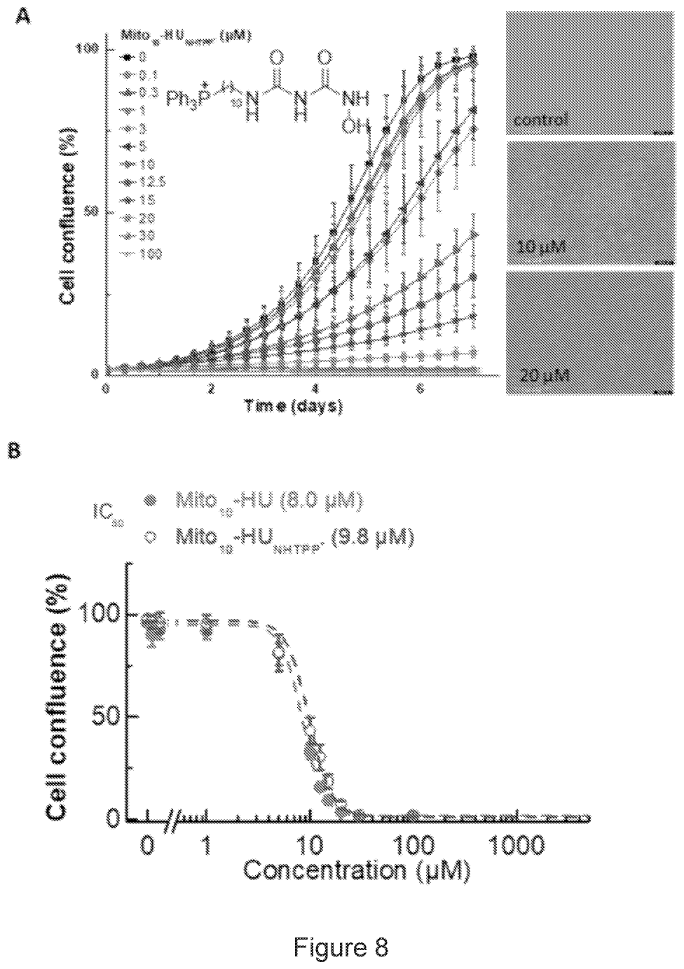

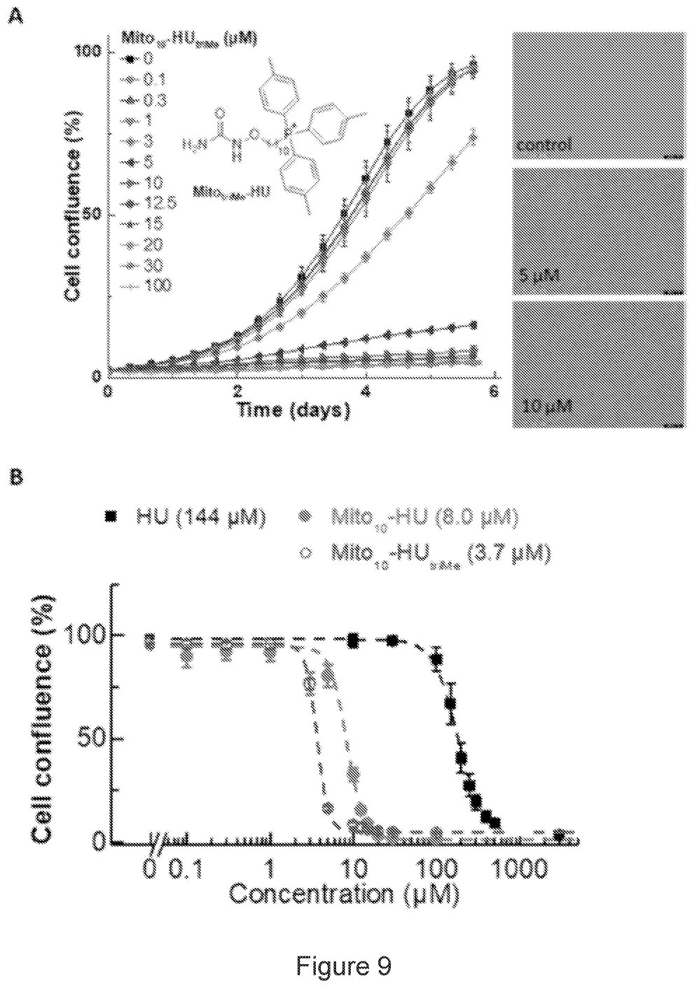

Furthermore, we provided additional results in support of our hypothesis that enhancing the hydrophobicity of mitochondria-targeted TPP⁺ compounds (without increasing the chain length) increases their antiproliferative effects. We synthesized new compounds in which the phenyl groups in Mito₁₀-HU were substituted by three methyl groups (e.g., Mito-HU_{triMe}). The hydrophobicity of Mito₁₀-HU (log P=6.7) is increased by the substitution of three methyl groups, Mito₁₀-HU_{triMe} (log P=8.3). As shown in FIG. 9, Mito₁₀-HU_{triMe} inhibited HCT116 cell proliferation at a lower concentration (IC₅₀=3.7 mM; log P=8.3) than Mito₁₀-HU (IC₅₀=8.0 mM; log P=6.7).

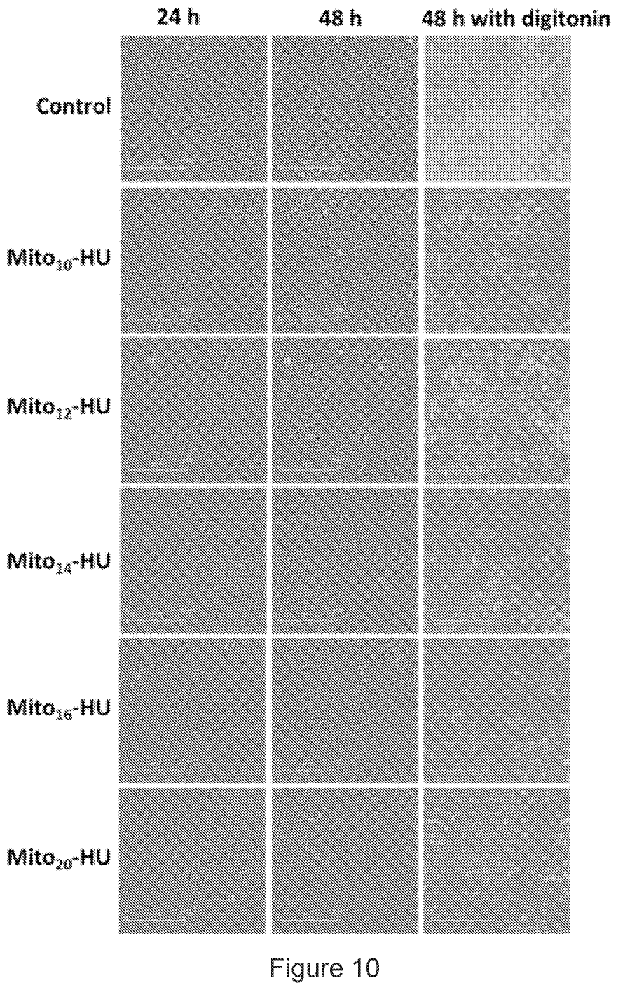

We used the SYTOX Green assay to monitor cell toxicity in an IncuCyte Live-Cell Analysis system. We tested the toxicity of Mito-HUs with varying hydrophobicities at their IC₅₀ concentrations obtained from cell proliferation results (FIG. 1B). A representative picture for Mito-HUs is shown in FIG. 10. Although a 50% inhibition of cell proliferation was observed at this concentration (FIG. 1 and Table 2), none of the Mito-HUs induced any apparent toxicity compared with control cells. These results suggest that the antiproliferative effects of Mito-HUs with varying side chain lengths are not due to nonspecific cellular toxicity. Permeabilization of cells with digitonin showed the total number of cells with or without Mito-HU treatments (FIG. 10). Results revealed that the total number of cells was decreased in Mito-HU-treated cells, consistent with the increased antiproliferative effects of Mito-HUs with increased hydrophobicity.

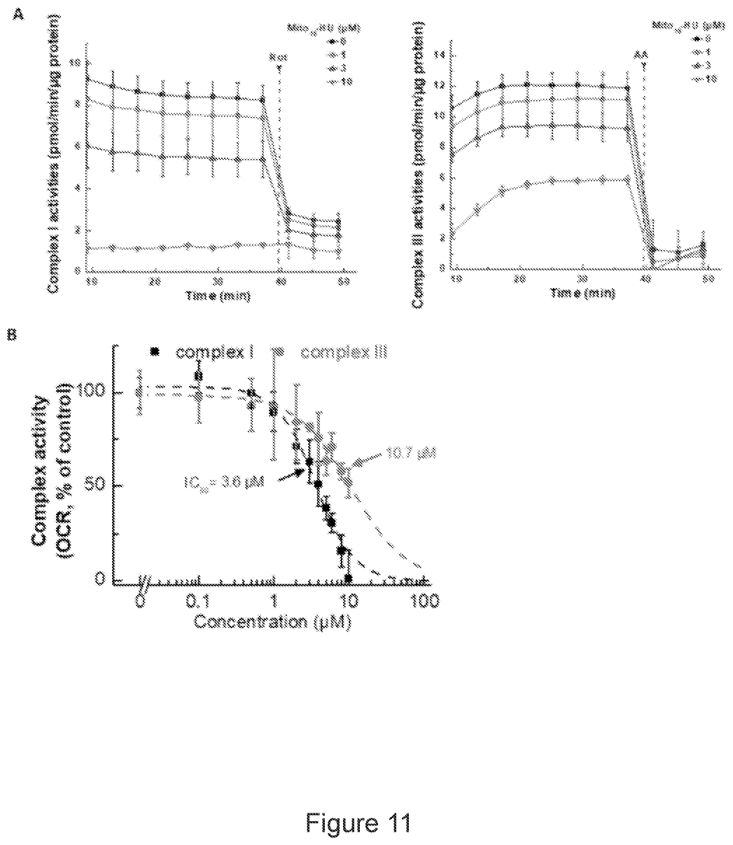

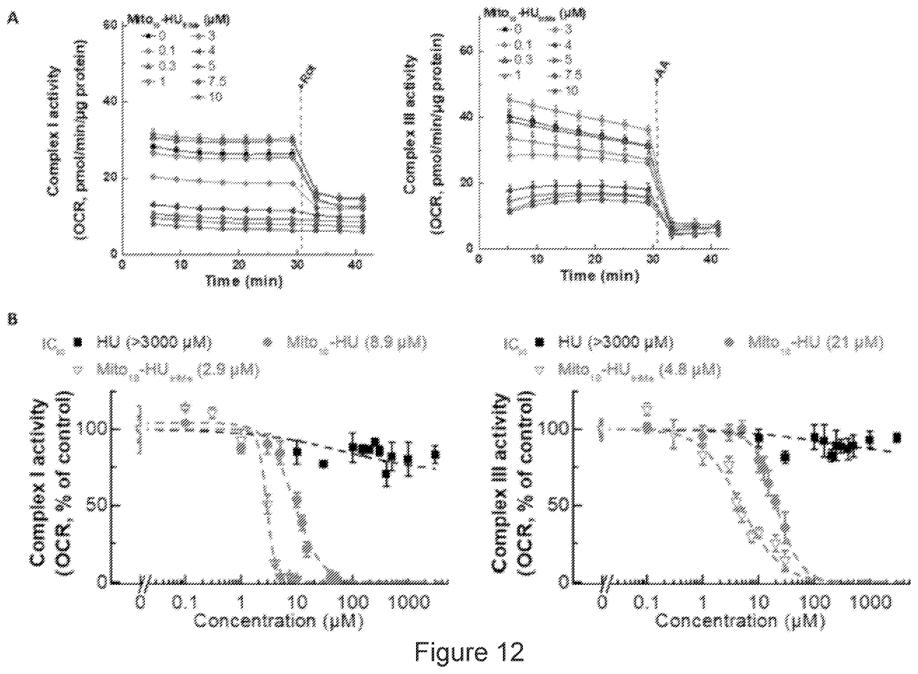

Mitochondrial complex activities were assessed by measuring the oxygen consumption rate (OCR) using the Seahorse technique (Cheng et al., 2013, 2016, 2019; Boyle et al., 2018; Fiorillo et al., 2016). We tested the efficacy of Mito-HUs with varying alkyl side chain lengths on complex I- and complex III-induced oxygen consumption. HCT116 cells were treated with Mito-HUs at different concentrations for 24 hr, and the complex I- or complex III-induced OCR was measured. As shown in FIG. 2A, Mito₁₀-HU effectively inhibited complex III-induced oxygen consumption and, more importantly, Mito₁₀-HU caused a significantly greater inhibition of mitochondrial complex I-driven oxygen consumption while HU had no apparent effects on either complex I- or complex III-driven oxygen consumptions. The IC₅₀ values for Mito₁₀-HU to inhibit oxygen consumption by complex I and complex III are 8.9 mM and 21 mM, respectively (FIG. 2B). Mito₁₀-HU, Mito₁₂-HU, Mito₁₄-HU, Mito₁₆-HU, and Mito₁₀-HU potently inhibited both mitochondrial complex I- and complex III-induced oxygen consumption in HCT116 cells (FIG. 2B). For comparison, MiaPaCa-2 cells also were treated with Mito₁₀-HU at different concentrations for 24 hr, and the complex I- or complex III-induced oxygen consumption was measured. Mito₁₀-HU inhibited complex I- and complex III-induced oxygen consumption in MiaPaCa-2 cells (FIG. 11); the IC₅₀ values to inhibit complex I- and complex III-induced oxygen consumption are slightly lower than those reported for HCT116 cells. Furthermore, Mito₁₀-HU_{triMe} inhibited complex I- and complex III-induced oxygen consumption more potently than Mito₁₀-HU in HCT116 cells (FIG. 12). This result is consistent with Mito₁₀-HU_{triMe} inhibiting HCT116 cell proliferation at a lower concentration, as shown in FIG. 9. Statistical analyses of results for complex I- and complex III-induced oxygen consumption as a function of different Mito-HUs (FIG. 2) are shown in Tables 4 and 5, respectively.

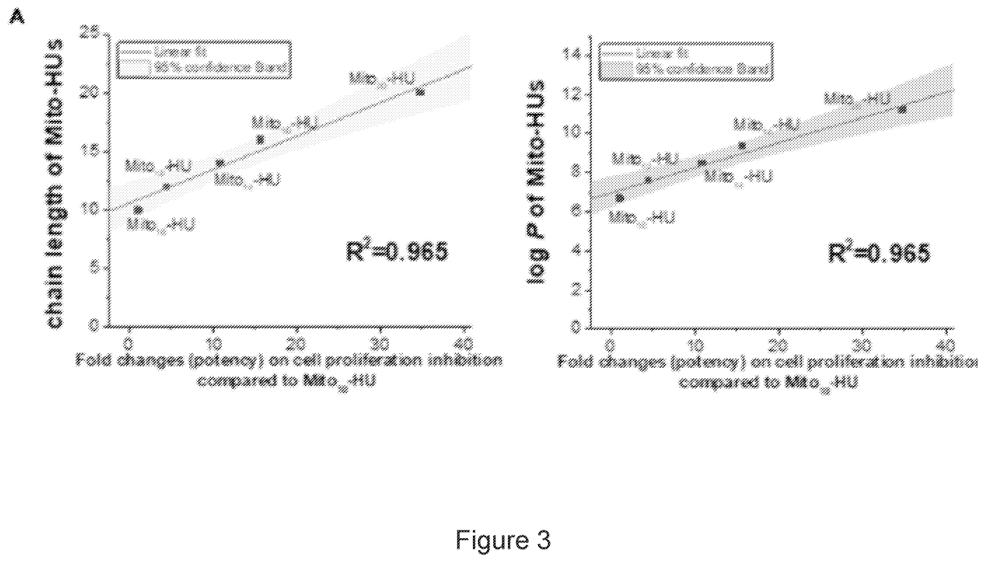

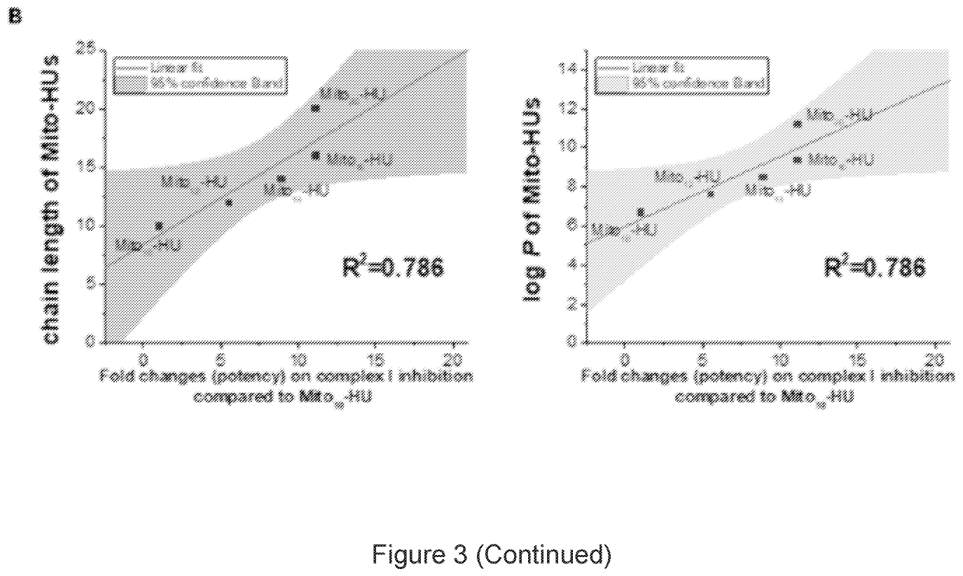

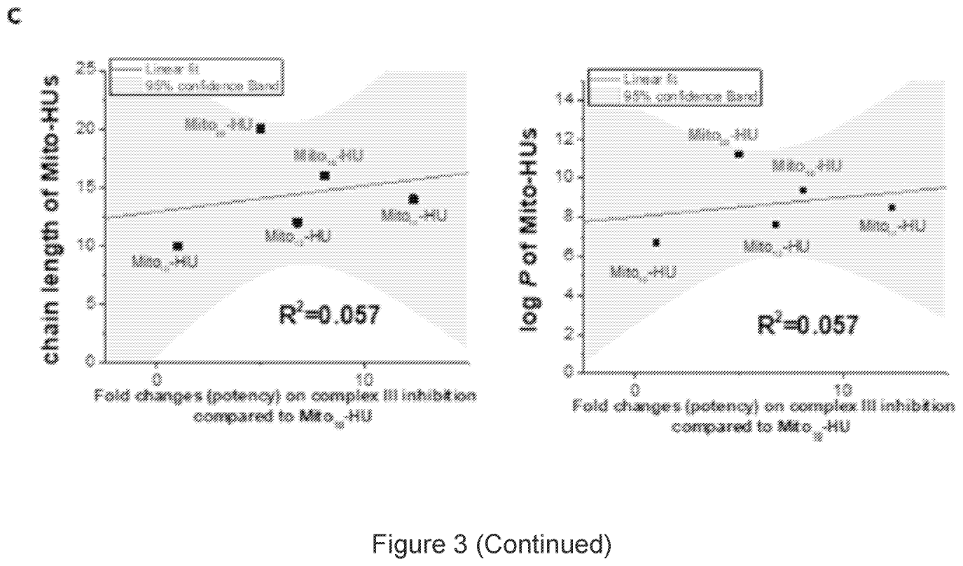

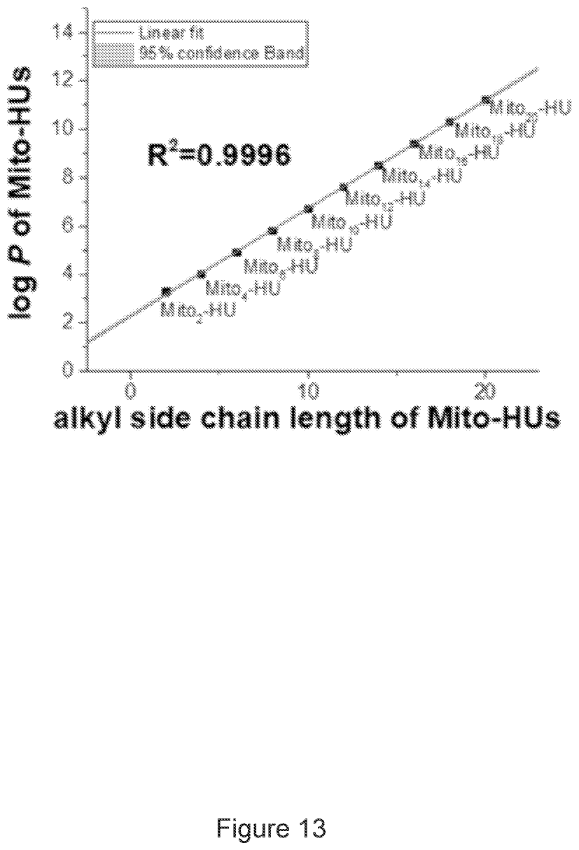

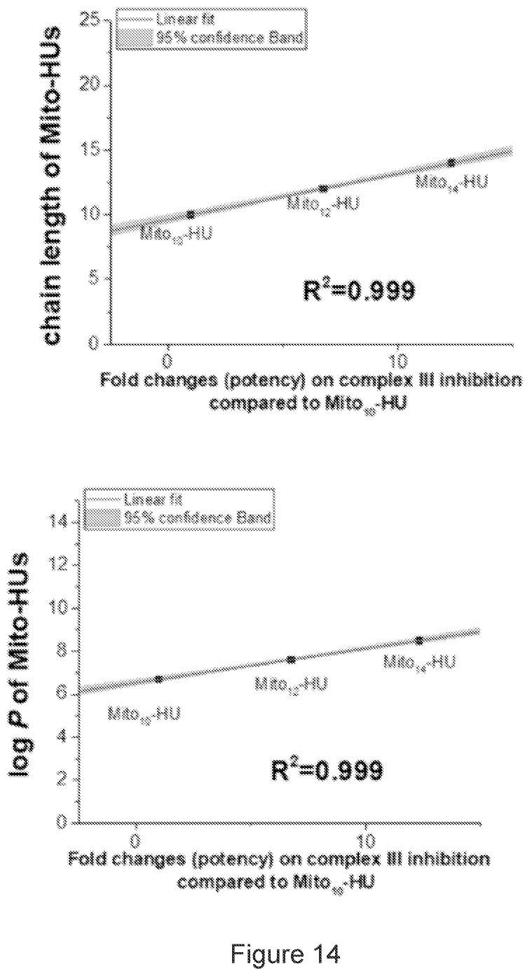

Next, we investigated in detail the relationship between the different Mito-HU analogs (FIG. 33) and the extent of inhibition effects of cell proliferation and mitochondrial complex activities. There is a strong linear fit (R²=0.9996, FIG. 13) between alkyl side chain length and relative hydrophobicity (log P, FIG. 33). First, we analyzed the correlation between the chain length and hydrophobicity of Mito-HUs and their antiproliferative effects (FIG. 3). FIG. 3A shows the relationship between the chain length (left) and hydrophobicity (right) as a function of a fold-increase in antiproliferative potency (compared with Mito₁₀-HU taken as unity). Results indicate an excellent degree of correlation (95% confidence band, R²=0.965) between increasing chain length/hydrophobicity and inhibition of proliferation in HCT116 cells treated with Mito-HUs, and that the antiproliferative effects of the Mito-HUs increase with the increasing hydrophobicity of the Mito-HUs. The correlation between the chain length/hydrophobicity and inhibition of mitochondrial complex I- and complex III-induced oxygen consumption revealed that the extent of inhibition of complex I- but not complex III-induced oxygen consumption is more linearly related to the increasing chain length and hydrophobicity of the Mito-HUs (FIGS. 3B and 3C). However, there is a good linear fit for the chain length of Mito-HUs up to 14 and inhibition of complex III-induced oxygen consumption (FIG. 14). These results indicate that the inhibitory effects of Mito-HUs on complex III are maximal at chain length 14, and further increasing the chain length decreases the effectiveness on complex III inhibition.

Furthermore, we determined the functional changes in mitochondria induced by Mito-HU. Treatment with Mito-HU and another drug that targets the monocarboxylate transporter in mitochondria synergistically inhibited proliferation (FIG. 15). A representative example is shown in FIG. 15A, using a concentration at which Mito₁₀-HU and syrosingopine, an inhibitor of MCT-1/MCT-4-mediated lactate transport, minimally affected cell proliferation. However, treatment with both Mito₁₀-HU and syrosingopine at these concentrations elicited a dramatic decrease in proliferation (FIG. 15A). The combination index values show that the dual inhibition of cell proliferation induced by Mito₁₀-HU and syrosingopine is synergistic (FIG. 15B). These results are consistent with the previous report on dual inhibition induced by metformin, a weak inhibitor of mitochondrial NADH dehydrogenase, and syrosingopine in cancer cells (Benjamin et al., 2018).

### Immunomodulatory Effects of Mito-HU with Varying Hydrophobicities

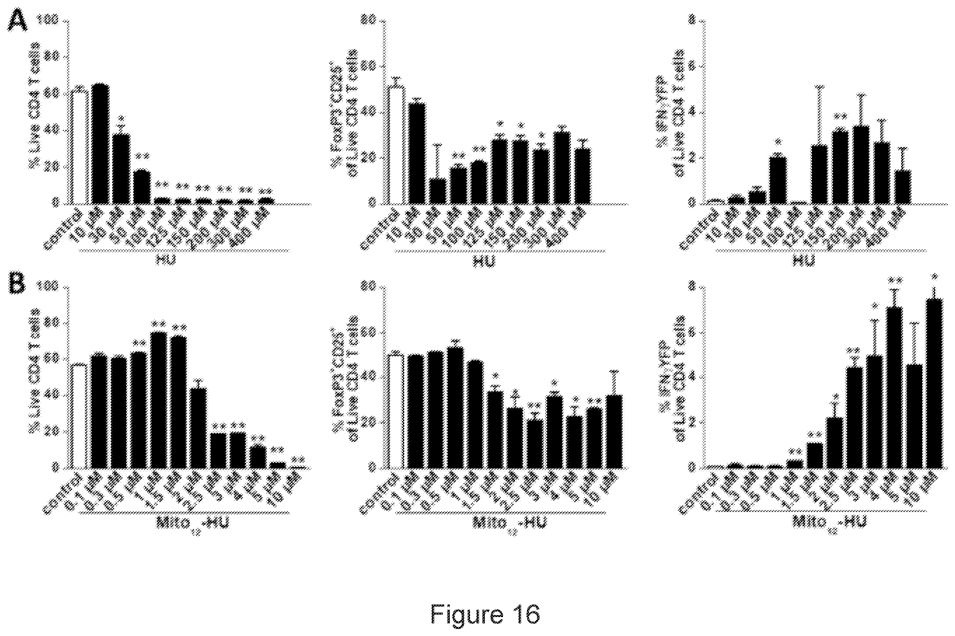

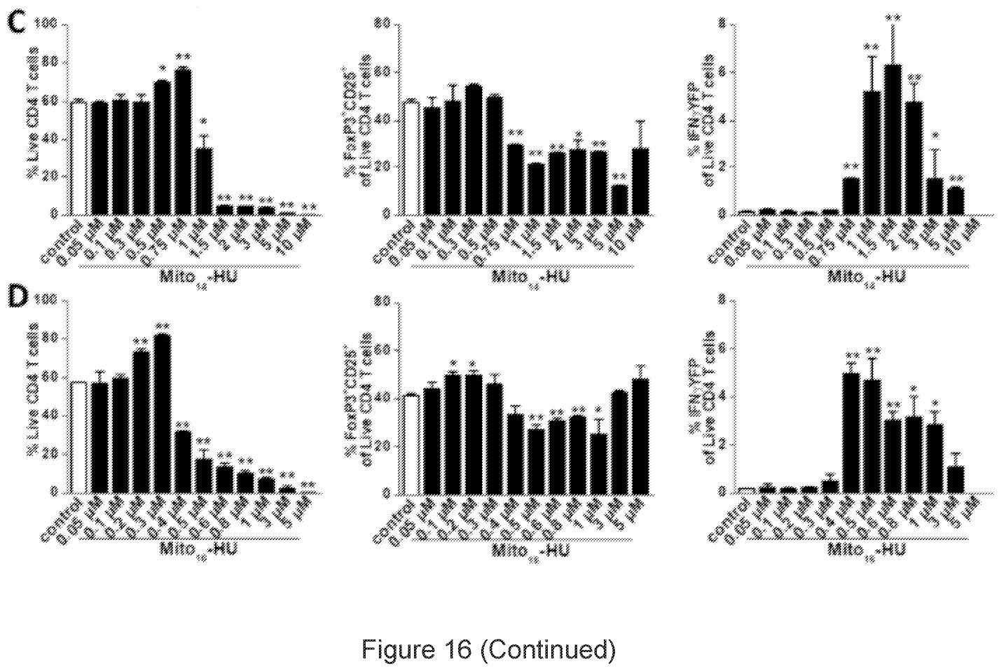

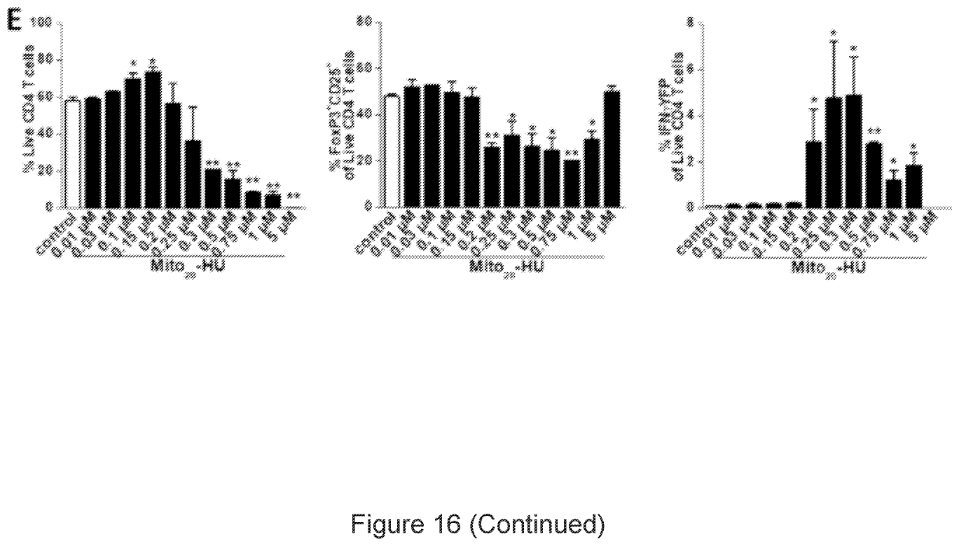

Published reports suggest that selective targeting and inhibition of mitochondrial complex III mitigate and reverse immunosuppression by T_{regs}, promoting the function of T_{eff} cells (Weinberg et al., 2019; Das et al., 2019). To investigate the effects of Mito₁₀-HU on T_{eff} versus T_{regs}, naive CD4⁺ T cells were isolated from SMARTA triple reporter mice and activated in vitro with GP₆₁₋₈₀ peptide (1 mg/mL) in the presence of TGFb (5 ng/mL) and IL-2 (100 ug/mL) to induce T_{reg} differentiation, as described in the STAR Methods section. The CD4⁺ T cells were treated with Mito₁₀-HU at varying concentrations. As shown in FIG. 4A, which depicts the gating strategies to assess these T cells, only live cells were gated out for further functional analysis. After six days, cells were stained to assess viability, phenotype, and function using flow cytometry. Results demonstrate that Mito₁₀-HU (FIGS. 4A-4C) inhibited FOXP3⁺ T_{reg} differentiation and/or survival and promoted T_{eff} IFNg production. Next, we investigated the effect of different Mito-HUs with varying hydrophobicities (Mito₁₂-HU, Mito₁₄-HU, Mito₁₆-HU, and Mito₂₀-HU (FIG. 16). Table 6 lists the concentrations of Mito-HUs presented in FIGS. 4B and 16 that have the maximum effect on IFNg-YFP. As shown in Table 6, the most hydrophobic Mito-HU (e.g., Mito₂₀-HU) elicited the maximal effect on T_{eff} activation at the lowest concentration (0.3 mM).

Next, we investigated the effects of different Mito-HUs on immunosuppressive cells found in the TME. MDSCs have been shown to suppress CD8 T cells using several mechanisms including elevated generation of reactive oxygen species (Kawano et al., 2015). MDSCs consist of two distinct subsets of cells: M-MDSCs, which are characterized by the surface markers CD11b⁺, F4/80⁻, Ly6C⁺, and Ly6G⁻, and suppressive neutrophils or polymorphonuclear MDSCs (PMN-MDSCs), which express the surface markers CD11b⁺, F4/80⁻, Ly6C⁺, and Ly6G⁺ in mice (Veglia et al., 2018). Intriguingly, suppressive neutrophils differentiate from a Ly6C⁺, Ly6G⁻, and cKit⁺ monocytic-like precursor of granulocytes primarily in the spleen (Mastio et al., 2019). A similar gating strategy employed for T cells was used in all MDSC differentiation assays, which were tested only on live cells after treatments. The results indicate the effects of Mito-HUs on the distributions/percentage populations of live cells at different stages of MDSC differentiation. The schematic representation of MDSC differentiation is shown in FIG. 5A. Mito₁₄-HU reduced the frequency of M-MDSCs (FIG. 5B). FIGS. 5B and 5C show that Mito₁₄-HU dose-dependently inhibited M-MDSC and suppressive neutrophils. Next, we investigated the effect of different Mito-HUs with varying hydrophobicities. FIG. 5D shows the dose-dependent effects of Mito₁₀-HU, Mito₁₂-HU, Mito₁₄-HU, Mito₁₆-HU, and Mito₂₀-HU on the frequency of M-MDSCs. The IC₅₀ values for the different Mito-HUs are 9.6 mM, 2.6 mM, 0.89 mM, 0.44 mM, and 0.44 mM, respectively (FIG. 5D). FIG. 5E shows the effect of Mito-HUs on suppressive neutrophils. More hydrophobic Mito-HUs (Mito₁₄-HU, Mito₁₆-HU) demonstrated greater inhibitory effects on suppressive neutrophils (FIG. 5E). These results suggest that more hydrophobic Mito-HUs inhibit both M-MDSCs and suppressive neutrophils.

### Discussion

In this study, we show that TPP⁺-conjugated Mito-HUs with varying alkyl side chain lengths potently increases their antiproliferative effects in colon cancer and other cancer cells. We also report that fine-tuning the hydrophobicity of mitochondria-targeted TPP⁺-conjugated compounds (Asin-Cayuela et al., 2004) could enhance their antiproliferative efficacy in tumor cells and modulate immune function. Results show that increasing the alkyl side chain to a 20-carbon analog (Mito₂₀-HU) inhibits the proliferation of HCT116 human colon cancer cells (IC₅₀=0.23 mM; log P=11.2) much more effectively than Mito₁₀-HU (IC₅₀=8.0 mM; log P=6.7). As shown in Table 2, substitution of the TPP⁺ group with methyl (—CH₃), trifluoromethyl (—CF₃), and chlorine (—Cl) substituents increases the hydrophobicity of Mito₁₀-HU. In future studies, it will be of interest to investigate the hydrophobic effect of the substituted TPP⁺ group on the mitochondrial targeting of complex I and complex III.

Reports indicate that mitochondria play an important role in modulating immune function (Weinberg et al., 2019). In the TME, cytotoxic T cells and antitumor M1 macrophages typically depend on glycolysis.

However, other cells in the TME—such as the immunosuppressive M2 macrophages, MDSCs, or T_{regs}—depend on mitochondrial functions including OXPHOS (Weinberg et al., 2019). Molecular profiling studies in melanoma patients revealed considerable immunosuppression and increased expression of OXPHOS genes in melanoma brain metastases compared with extracranial metastases (Fischer et al., 2019). Under these conditions, the OXPHOS inhibitor IACS-010759 effectively blocked the formation of metastases. In the present study, we show that Mito-HUs inhibited MDSCs and that increasing the hydrophobicity of Mito-HU dose-dependently enhanced the inhibition of MDSCs (FIG. 5). The IC₅₀ value for Mito₂₀-HU to inhibit M-MDSCs is 0.44 mM as compared with 9.6 mM for Mito₁₀-HU (FIG. 5). These findings show that TPP⁺ containing mitochondria-targeted compounds exhibits similar dose-response effects in cancer cells and in immunosuppressive cells in the TME.

The TME provides a proinflammatory environment that facilitates tumor growth. High levels of reactive oxygen and nitrogen species generated potentially by MDSCs in the TME are cytotoxic to T cells and, thus, cripple their ability to destroy tumor cells (Corzo et al., 2009; Groth et al., 2019). T_{regs} suppress antitumor immunity that essentially hampers immunotherapy. Drugs inhibiting mitochondrial complex III have been shown to reverse the immunosuppressive function of T_{regs} (Weinberg et al., 2019). Because hydrophobic Mito-HUs can inhibit both complex I- and complex III-induced mitochondrial oxygen consumption, we tested whether Mito-HU can inhibit the T_{regs} population while increasing the population of T_{effs}. As shown in FIG. 4, Mito₁₀-HU inhibited T_{reg} differentiation and/or survival while stimulating T_{effs}. Reactive oxygen and nitrogen species generated by MDSCs have been shown to suppress the function and proliferation of T cells (Feng et al., 2018). Both NADPH oxidase 2, a source of superoxide, and inducible nitric oxide synthase, a source of nitric oxide, have been implicated in T cell suppression. Previous studies showed that administration of mitochondria-targeted drugs (e.g., Mito-apocynin) in MitoPARK mice inhibited the expression of NADPH oxidase 2 and inducible nitric oxide synthase, as well as the oxidative and nitrative damage in mitochondrial proteins and inflammation in glial cells (Langley et al., 2017). Mitochondria-targeted honokiol suppressed the phosphorylation of mitoSTAT3 (Pan et al., 2018). STAT3 (signal transducer and activator of transcription 3) is involved in the immunosuppression observed in pancreatic cancer patients (Trovato et al., 2019). Future studies will focus on testing these mechanisms for Mito-HUs. It is likely that modulating the hydrophobicity of Mito-HUs may play a key role in activating the antitumor immune response.

Depending on the alkyl side chain length, the calculated hydrophobicity, or the lipid-to-water partition coefficients (log P), of Mito-HU varies (FIG. 33). Because HU is very water-soluble (log P=−1.8), Mito₄-HU containing a four-carbon alkyl side chain is still hydrophilic (log P=4.0) and a substantial increase in hydrophobicity was noticeable only in Mito-HUs with a much longer alkyl chain with n R 10 (e.g., Mito₁₀-HU, Mito₁₆-HU, Mito₂₀-HU) (FIG. 33). Results show that with increasing hydrophobicity, the antiproliferative potency of Mito-HUs dramatically increased (FIG. 1 and Table 3). In contrast to HU, which had a negligible effect on mitochondrial oxygen consumption, Mito-HUs (Mito₁₀-HU, Mito₁₂-HU, Mito₁₆-HU, and Mito₂₀-HU) inhibited both mitochondrial complex I- and complex III-induced oxygen consumption at low micromolar concentrations (FIG. 2 and Tables 4 and 5).

Mito₄-HU, with a TPP⁺ moiety containing a four-carbon side chain, is anomalous in that its antiproliferative efficacy is lower than that of HU. In contrast, attaching a four-carbon alkyl side chain to a hydrophobic molecule has previously been shown to substantially increase its hydrophobicity and antiproliferative efficacy (Cheng et al., 2020a). HU exerts its cytostatic mechanism of action primarily through inhibition of RDR in proliferating cells. HU inhibits the tyrosyl radical that is required for RDR activity (Lassmann et al., 1992). The tyrosyl radical in RDR also participates in a long-range electron transfer to a cysteinyl residue located on the surface of RDR, forming a cysteinyl thiyl radical (Zhang et al., 2005; Chang et al., 2004), and it is likely that HU reacts with the radical on the surface and inhibits the activity.

The free radical mechanism is not viable once the ⁻OH group is derivatized. Thus, the antiproliferative mechanism of action of Mito-HUs differs from that of HU. Because HU is intrinsically very hydrophilic, it is necessary to conjugate HU with TPP⁺ containing much longer alkyl side chain lengths to increase the hydrophobicity of Mito-HUs as shown: Mito₁₀-HU (log P=6.7), Mito₁₂-HU (log P=7.6), Mito₁₆-HU (log P=9.4), and Mito₂₀-HU (log P=11.2).

Previous reports suggest that replacing the ⁻OH group in HU with an alkyl group inhibits its antiproliferative effect due to the lack of a tyrosyl or cysteinyl radical scavenging mechanism (Singh and Xu, 2016). However, replacing the ⁻OH group with alkyl-substituted TPP⁺ potently stimulates the antiproliferative mechanism that is dependent on the alkyl side chain length. Results show that Mito₄-HU (log P=4.0) did not inhibit the proliferation of MiaPaCa-2 cells, whereas Mito₁₀-HU (log P=6.7), Mito₁₂-HU (log P=7.6), Mito₁₆-HU (log P=9.4), and Mito₂₀-HU (log P=11.2) with increasing hydrophobicity exhibited increasing antiproliferative potencies. This suggests that replacement of the ⁻OH group with the hydrophobic cationic substituent alters their mechanism of action in cancer cells.

The radiation sensitizing property of HU is well established (Leyden et al., 2000). HU arrests cells in the S-phase by inhibiting the R2 subunit of the ribonucleotide reductase and DNA synthesis (Zhou et al., 2013; Yarbro, 1992). The activity of topoisomerases is maximal during DNA replication. Radiation inhibits DNA replication, inhibits DNA repair, and is more effective in tumor cells treated with a cytostatic agent.

Mitochondria-targeted drugs are also effective hypoxic radiation sensitizers (Chang et al., 2004). This effect, however, arises from inhibiting mitochondrial respiration. Radiation is not very effective under hypoxic conditions in eradicating tumor cells. Inhibiting mitochondrial respiration can effectively increase the oxygen concentration in hypoxic regions of tumor cells. As a result, inhibition of mitochondrial respiration has been shown to augment the efficacy of radiation therapy (Cheng et al., 2016; Ashton et al., 2018). Thus, combining Mito-HUs with radiation may be more effective in killing hypoxic tumor cells.

The immunomodulatory effects of Mito-HU are dependent on the alkyl side chain length. More hydrophobic Mito-HUs inhibited M-MDSCs and suppressive neutrophils at submicromolar concentrations (FIG. 5). The potency of inhibition increased with increasing side chain lengths. Mito-HUs inhibited proliferation of the colon cancer cells and M-MDSCs with a similar dose dependency. This class of mitochondria-targeted and structurally modified FDA-approved antitumor drugs could also play an important role in inhibiting metastatic tumors exhibiting enhanced immunosuppression and OXPHOS.

### Materials and Methods

### Cell Lines

The following cell lines were obtained from the American Type Culture Collection (Manassas, VA), where they were regularly authenticated: HCT116 (ATCC Cat #CRL-247, human colon cancer cells) and MiaPaCa-2 (ATCC Cat #CRL-1420, human pancreatic cancer cells). All cells were cultured in a humidified incubator at 37° C. and 5% carbon dioxide. HCT116 cells were maintained in RPMI 1640 medium (Thermo Fisher Scientific, Cat #11875), supplemented with 10% fetal bovine serum. MiaPaCa-2 cells were maintained in Dulbecco's Modified Eagle Medium (Thermo Fisher Scientific, Waltham, MA; Cat #11965) supplemented with 10% fetal bovine serum. All cells were stored in liquid nitrogen and used within 20 passages after thawing.

### Animals

SMARTA triple reporter mice were generated in the following manner. First, IL-10 and IL-21 double-reporter mice (Xin et al., 2018) were generated by cross-breeding IL-21-tRFP mice (kindly provided by Dr. Joseph Craft, Yale University) (Xin et al., 2015; Weinstein et al., 2016; Shulman et al., 2014) with 10 BiT mice (kindly provided by Dr. Casey Weaver, University of Alabama at Birmingham). Double reporter mice were crossed with GREAT (interferon-gamma reporter with endogenous polyA transcript) mice (Reinhardt et al., 2009) from Jackson Laboratory (Bar Harbor, ME; Cat #017581). These triple-reporter mice were then crossed with SMARTA mice (kindly provided by Dr. Dorian McGavern, National Institutes of Health) (Oxenius et al., 1998). Eight to 12-week-old mice were used to generate T_{reg} cultures. Mice were bred and maintained in a closed breeding facility, and mouse handling conformed to the requirements of the Medical College of Wisconsin Institutional Animal Care and Use Committee guidelines. All experimental protocols were approved by the Medical College of Wisconsin Institutional Animal Care and Use Committee.

### Syntheses of Mito-HUs

The Mito-HU derivatives were prepared by reacting HU with different halogeno-alkyltriphenylphosphonium salts in the presence of potassium carbonate at 70° C. in N,N-dimethylformamide (DMF). In addition, the negative control, decyl-HU, was prepared by reacting bromodecane with HU using the same experimental conditions. To assess the relative hydrophobicities of HU and Mito-HUs, the octanol/water partition coefficients (log P) were calculated using a QSAR (quantitative structure-activity relationship) analysis and rational drug design (FIG. 33). This method also uses a consensus model built with ChemAxon software (San Diego, CA) (Viswanadhan et al., 1989; Klopman et al., 1994). FIG. 33 lists the log P values along with the calculated regions of the relative hydrophilic and hydrophobic regions. As shown in FIG. 33, there was a significant increase in the hydrophobicity of Mito-HU (as compared with HU) with the increasing alkyl side chain length (from C-4 to C-20).

General. All chemicals and organic solvents were commercially available and were used as supplied. The reactions were monitored by TLC using silica gel Merck ⁶⁰F254. Crude materials were purified by flash chromatography on Merck Silica gel 60 (0.040-0.063 mm) or on a RediSep reverse phase C18 column. ³¹P nuclear magnetic resonance (NMR), ¹H NMR and ¹³C NMR spectra were recorded at 400.13 MHz spectrometers and 75.54 MHz, respectively. 1H NMR spectra were recorded using a Bruker DPX AVANCE 400 spectrometer (Marseille, PACA, France) equipped with a quattro nucleus probe. Chemical shifts (d) are
reported in ppm and J values in Hertz.

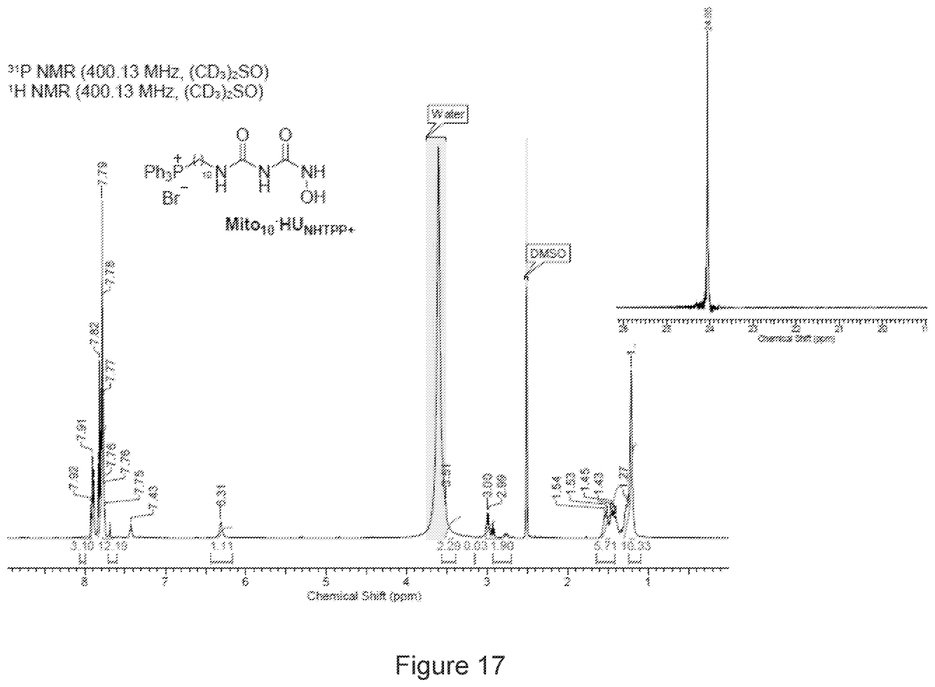

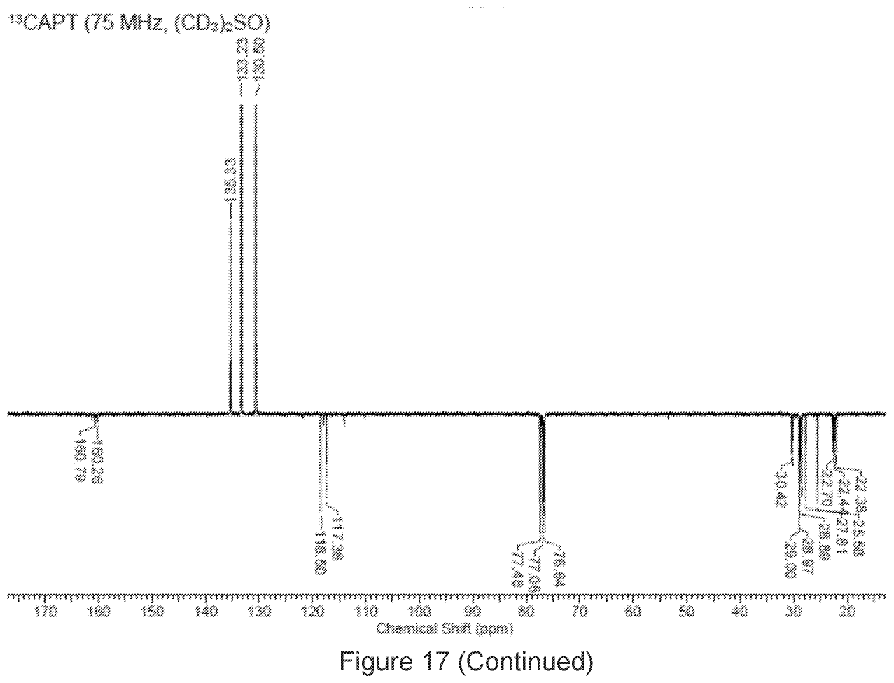

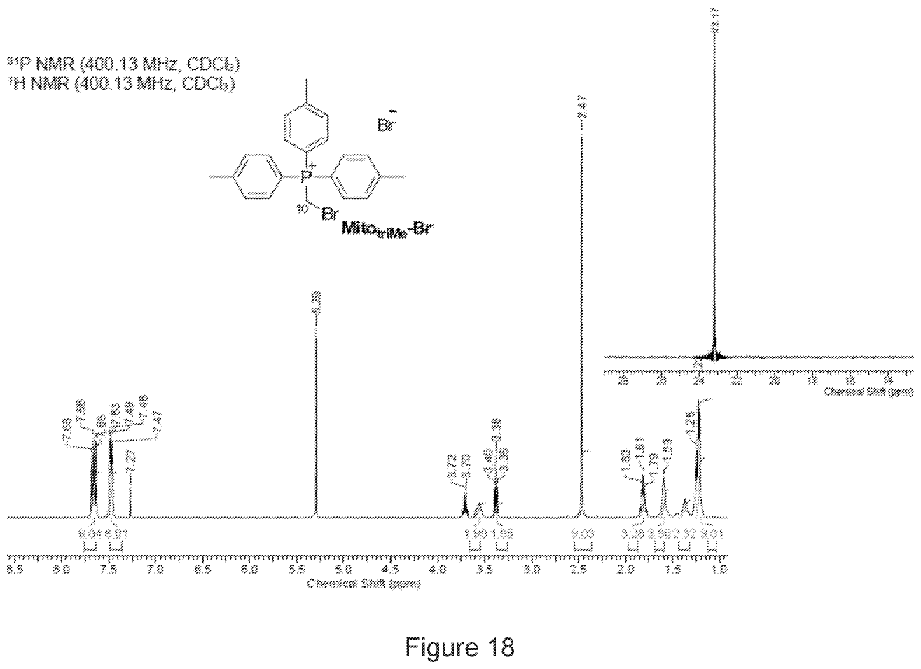

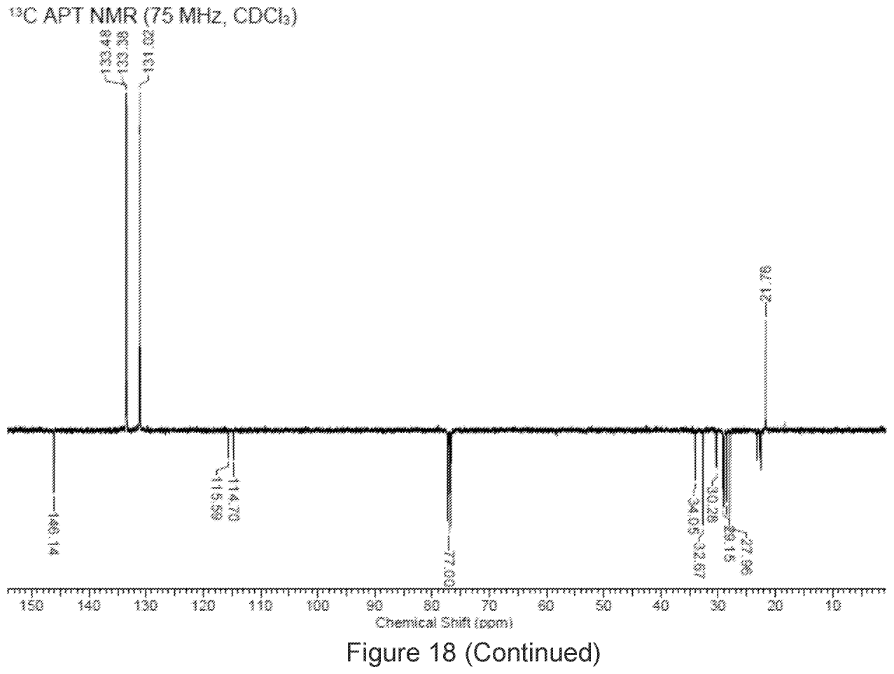

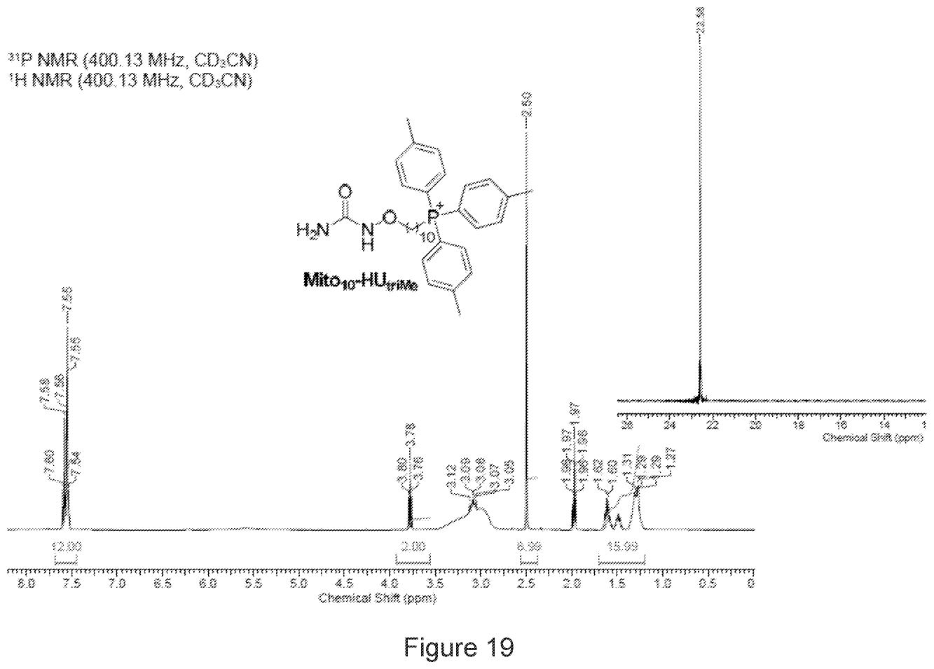

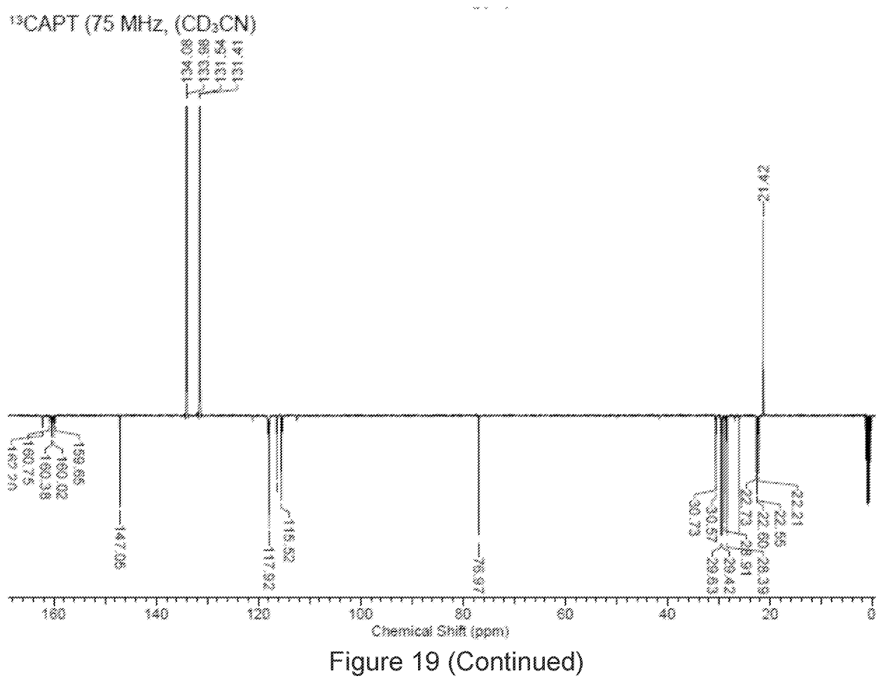

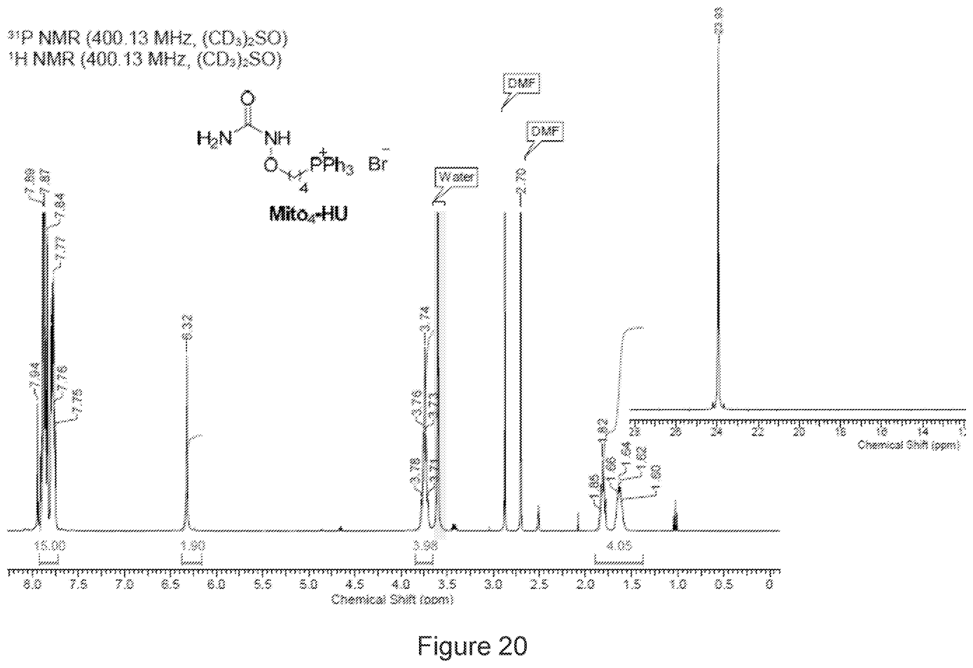

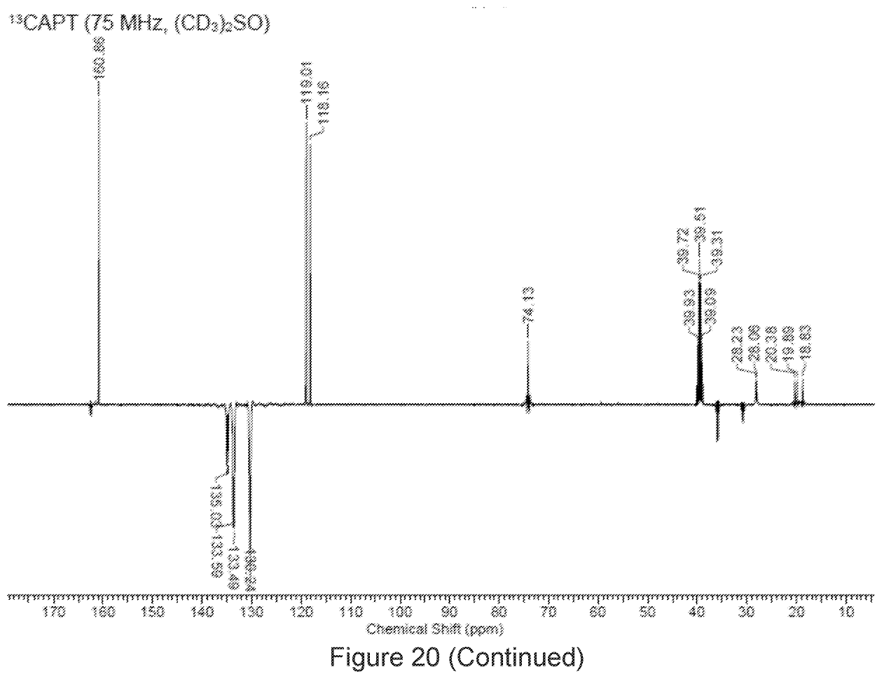

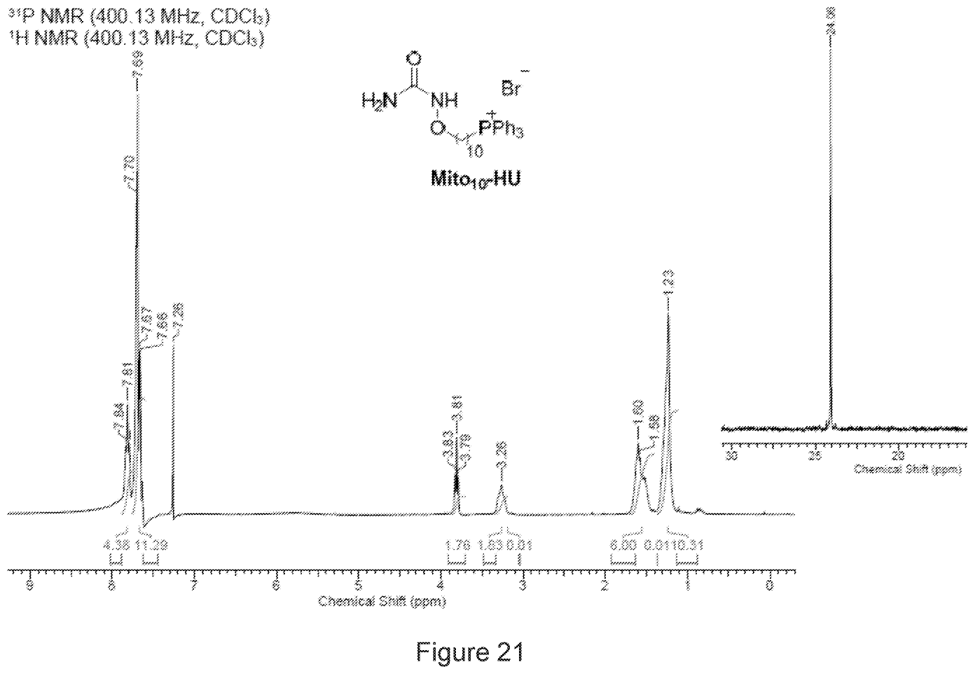

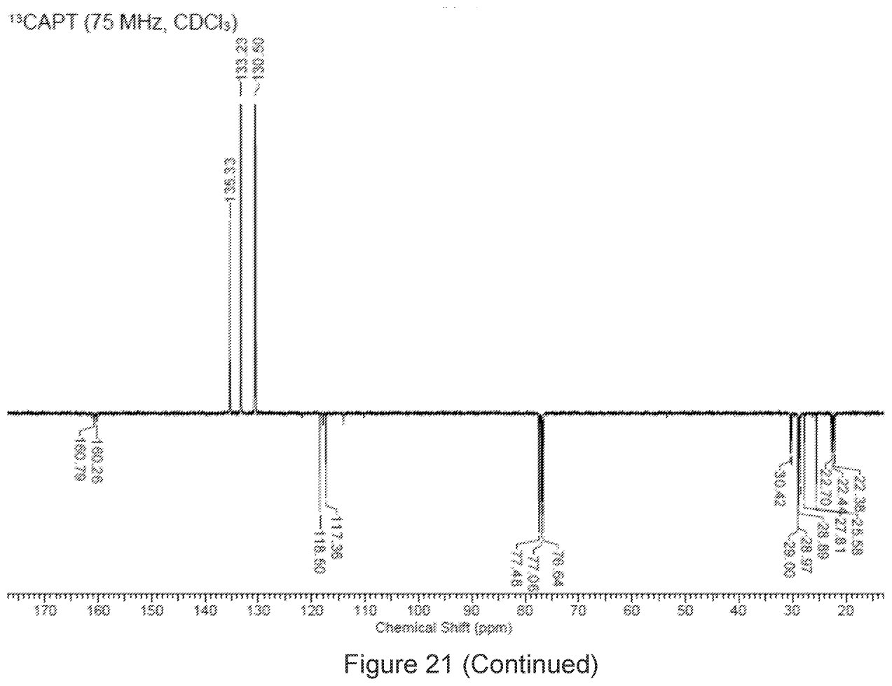

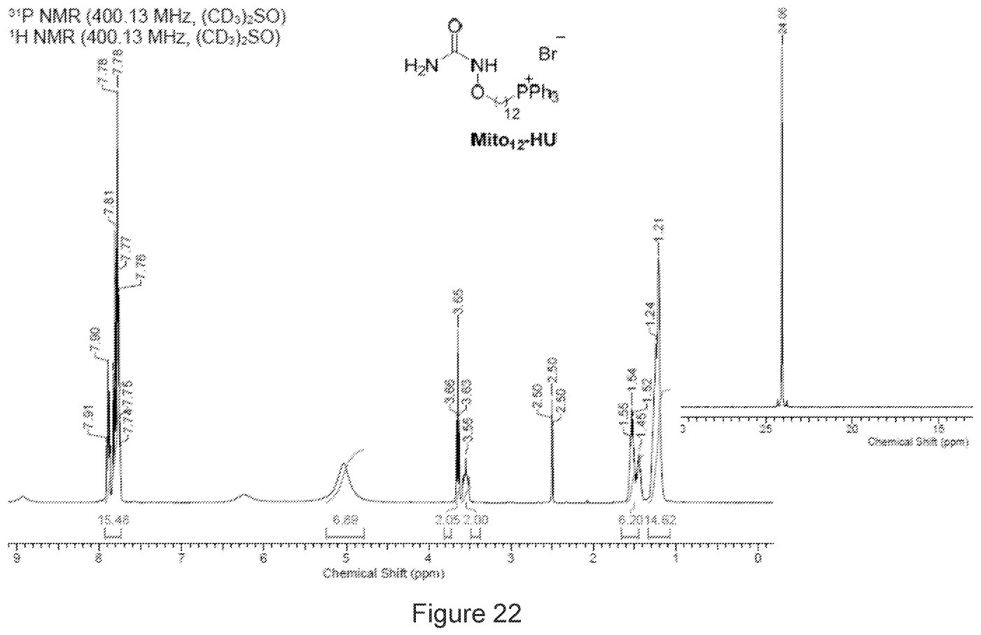

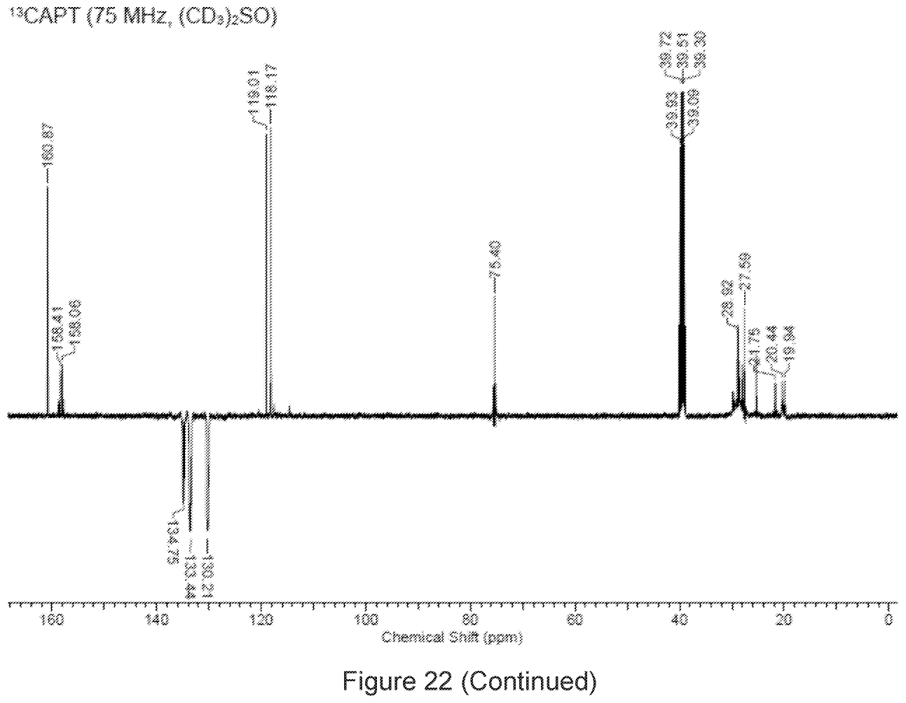

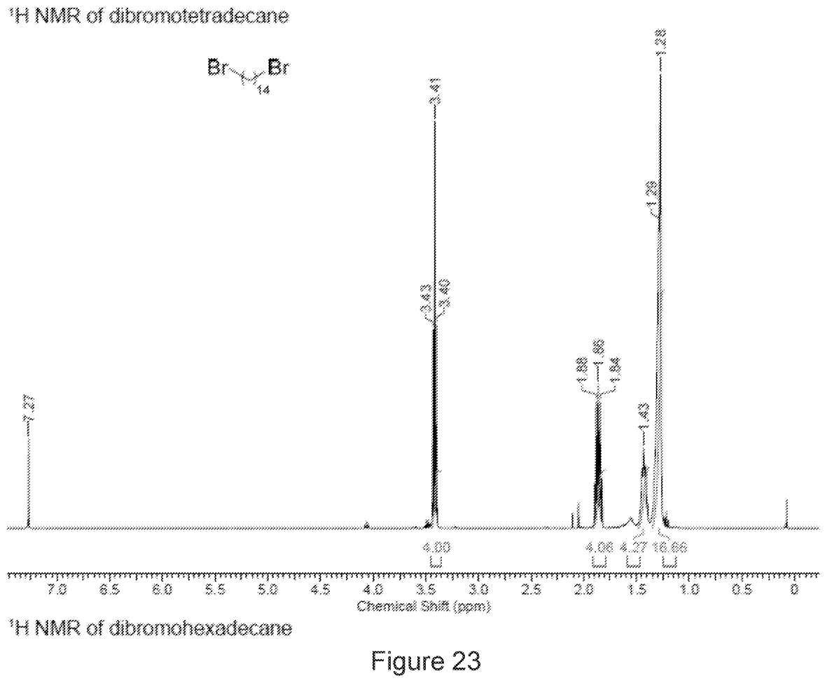

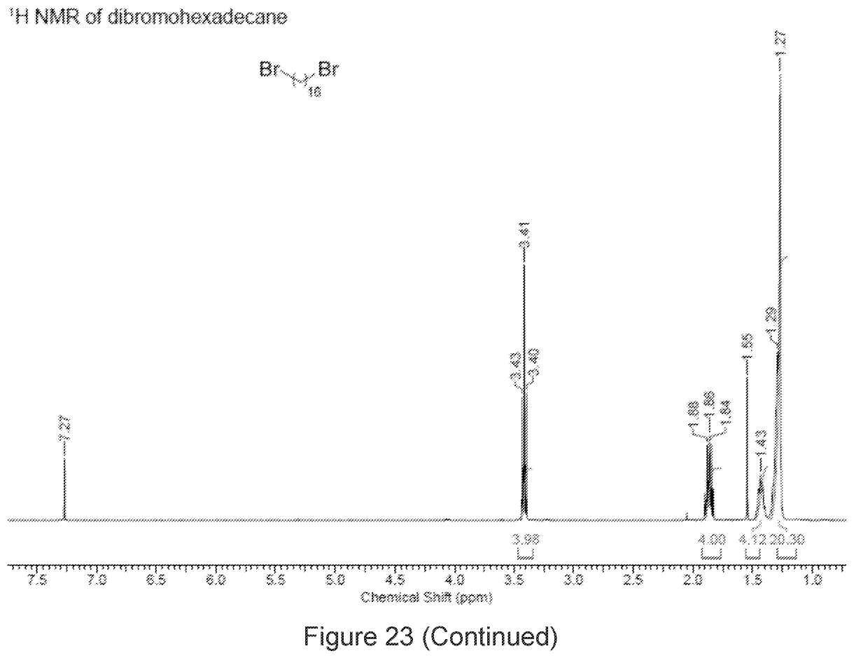

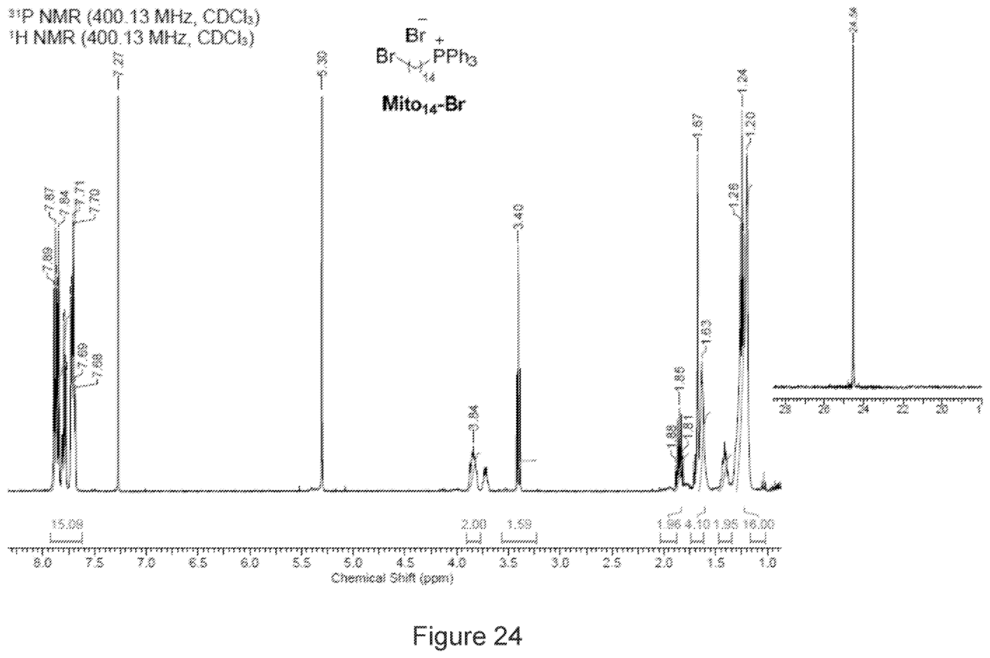

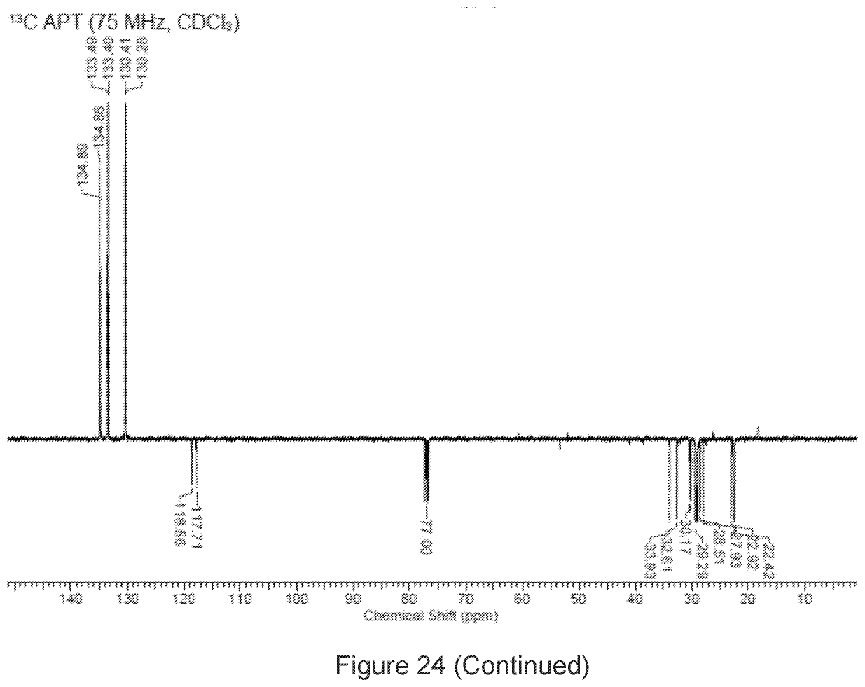

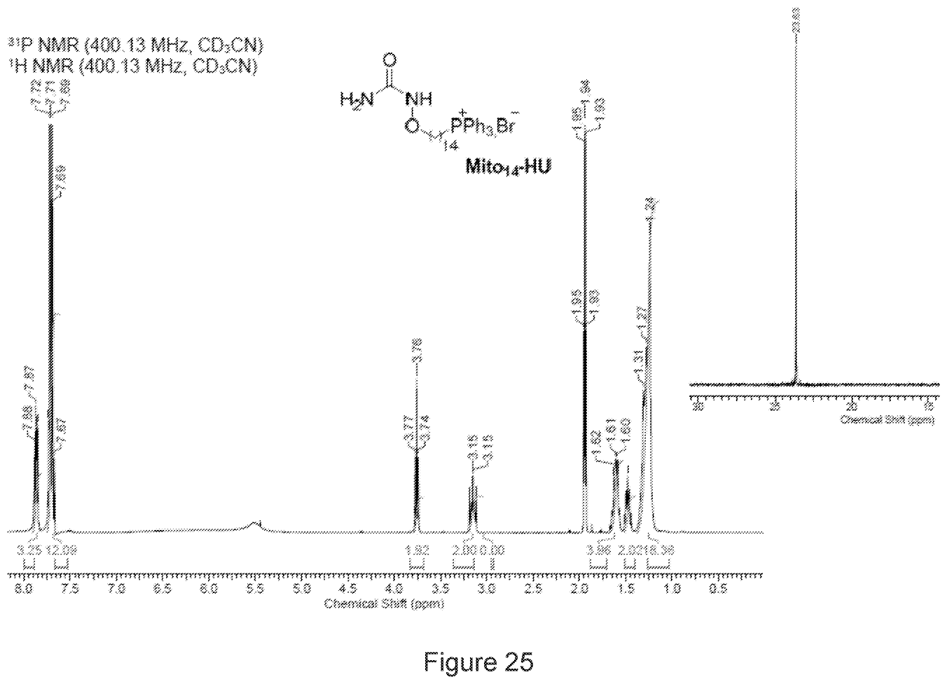

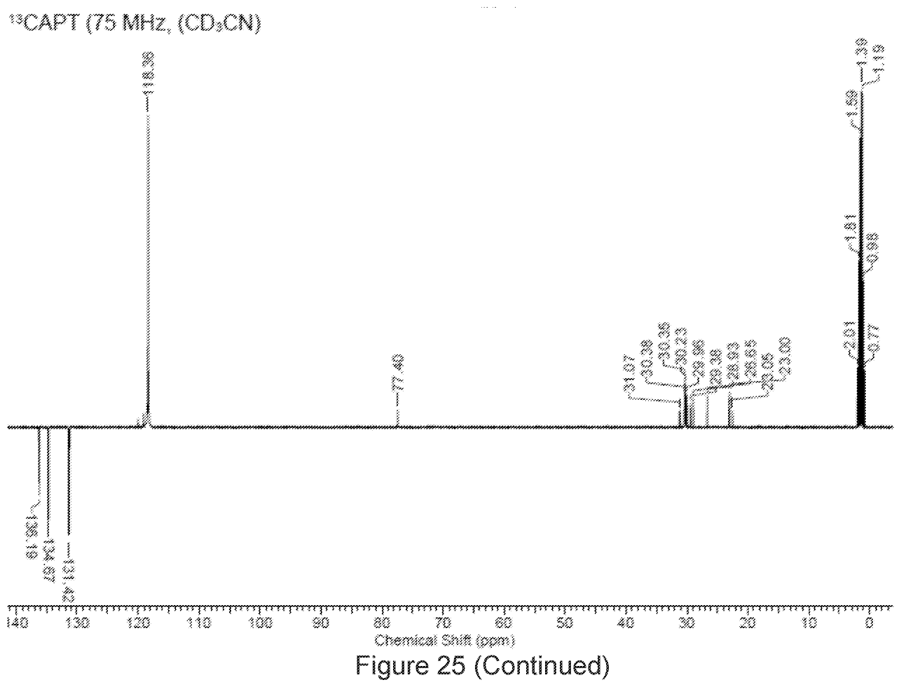

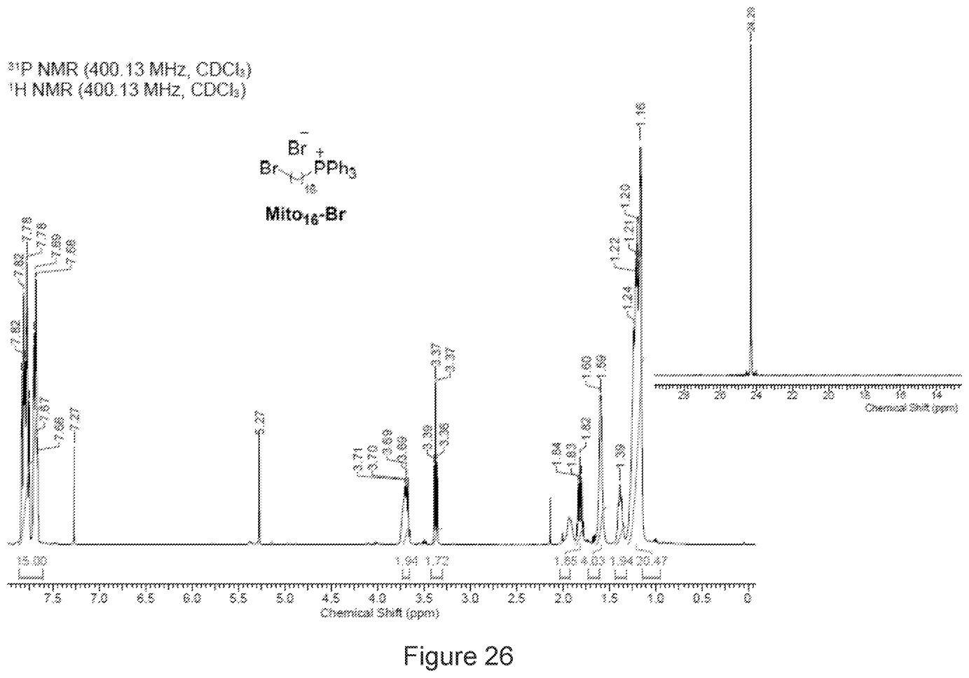

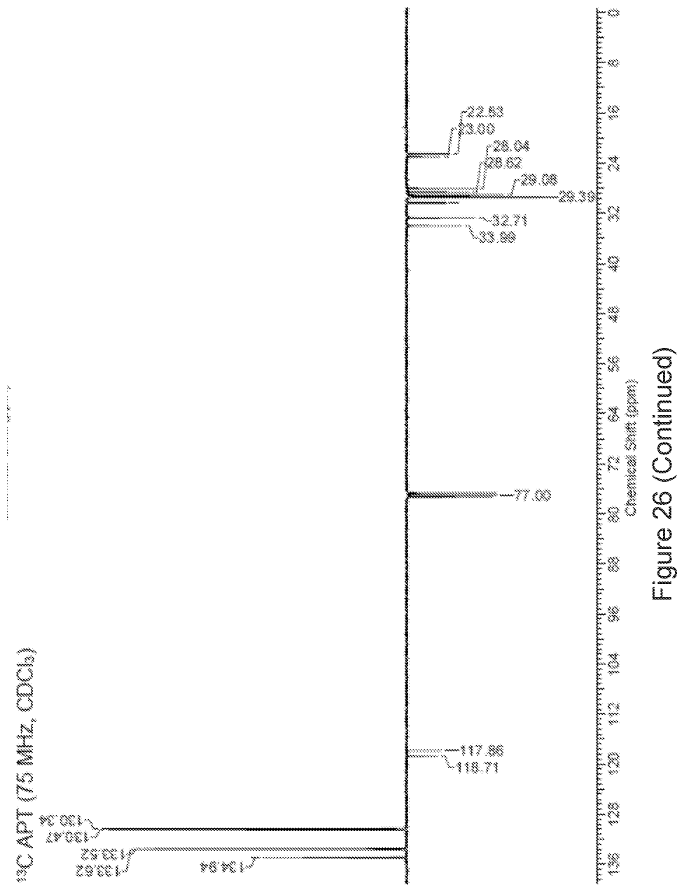

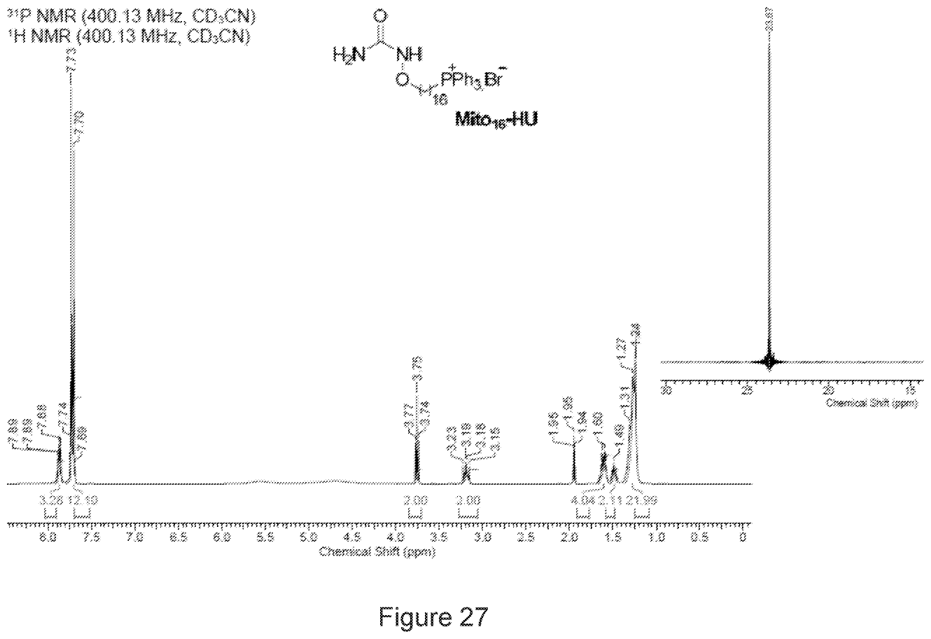

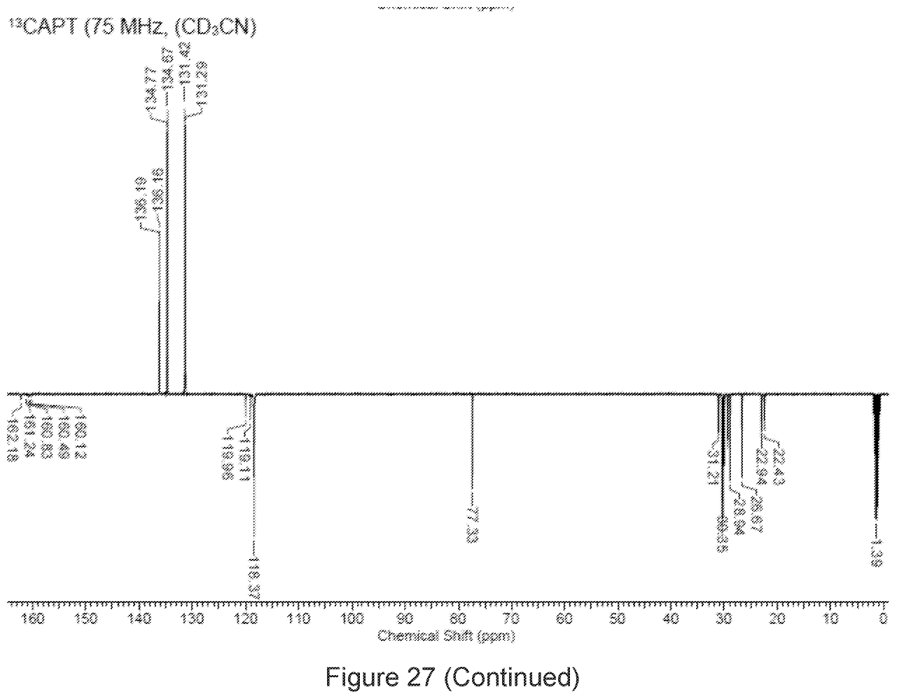

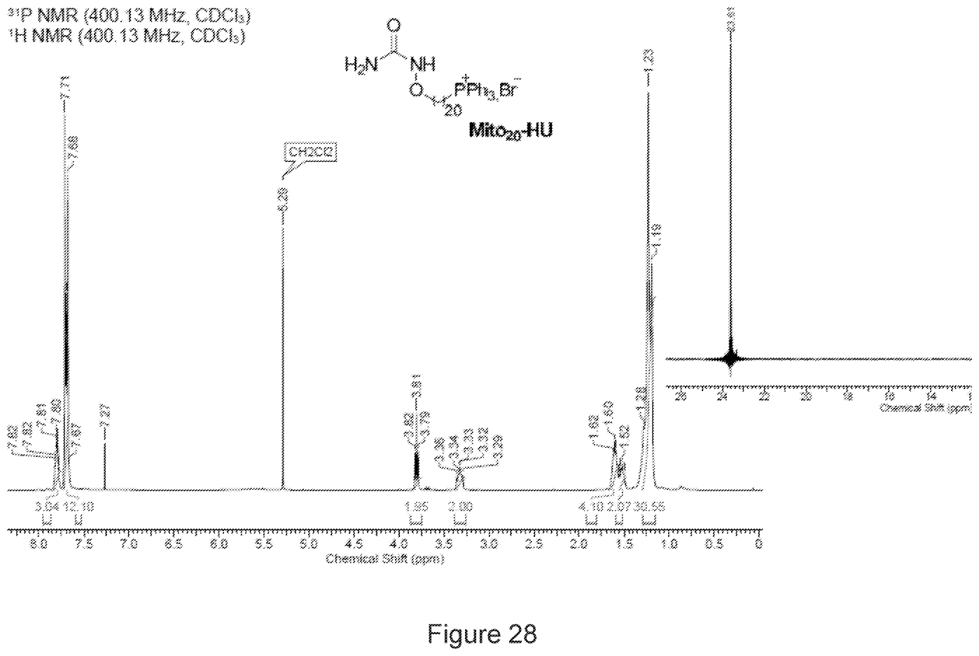

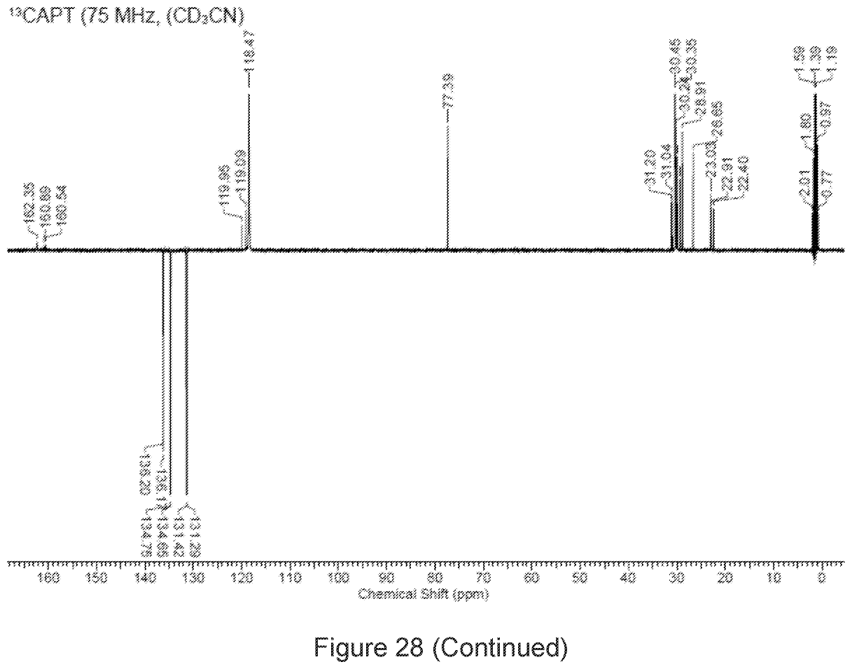

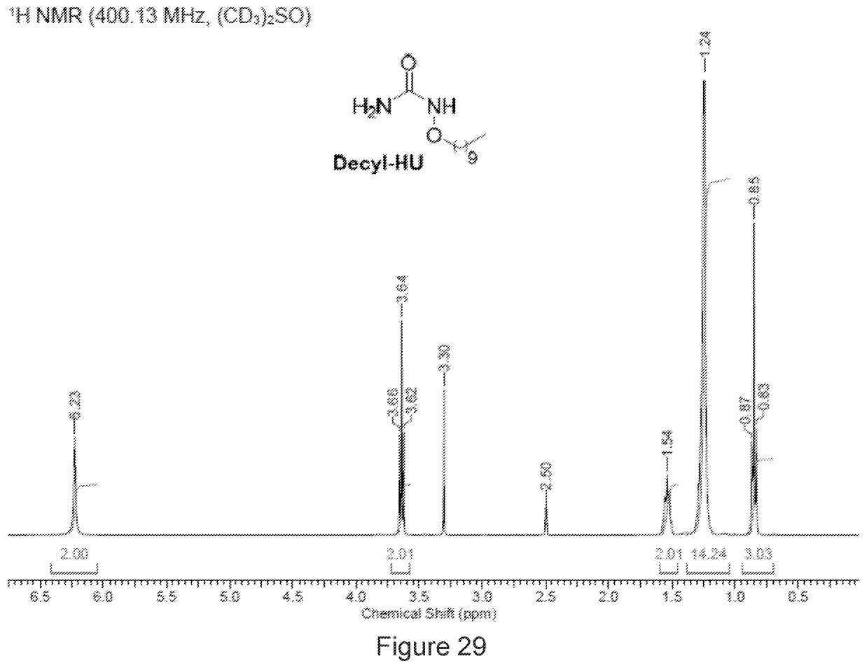

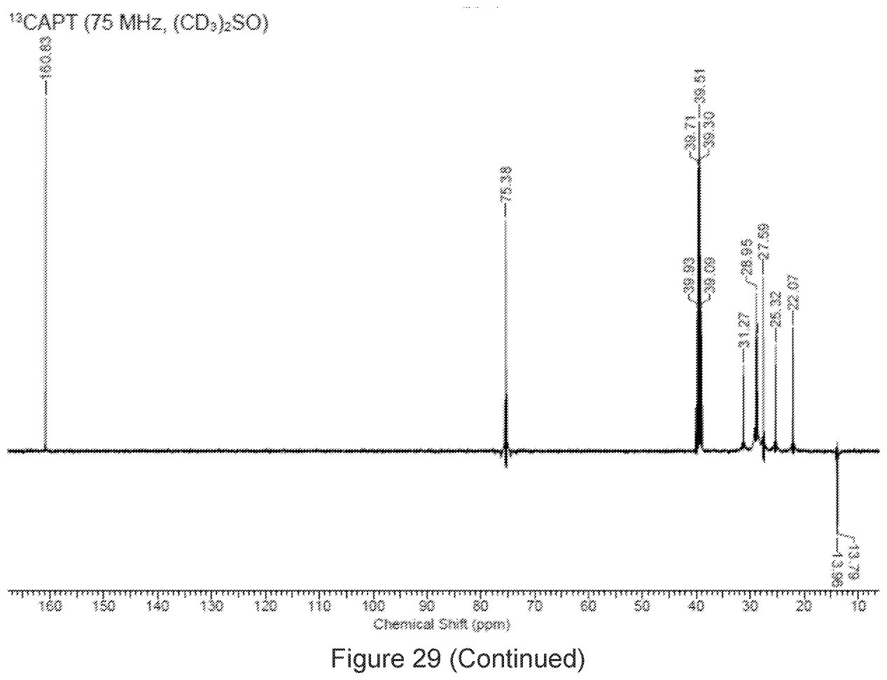

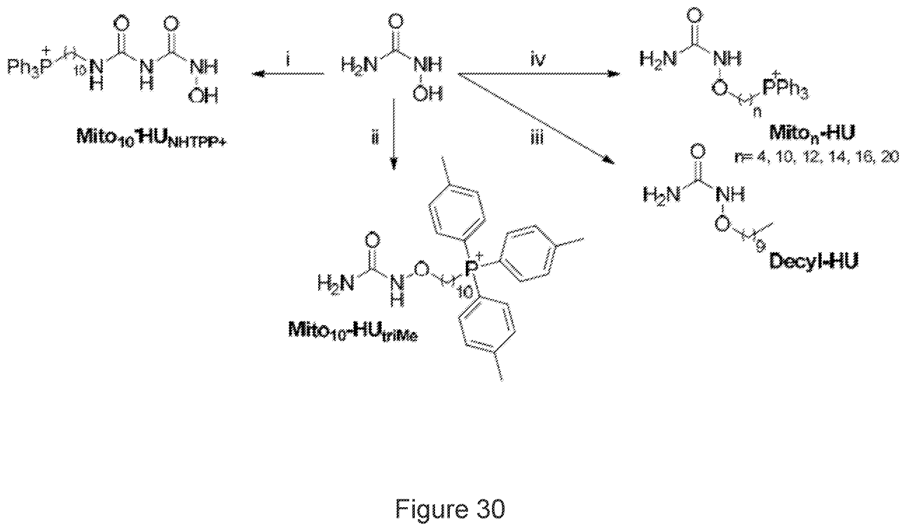

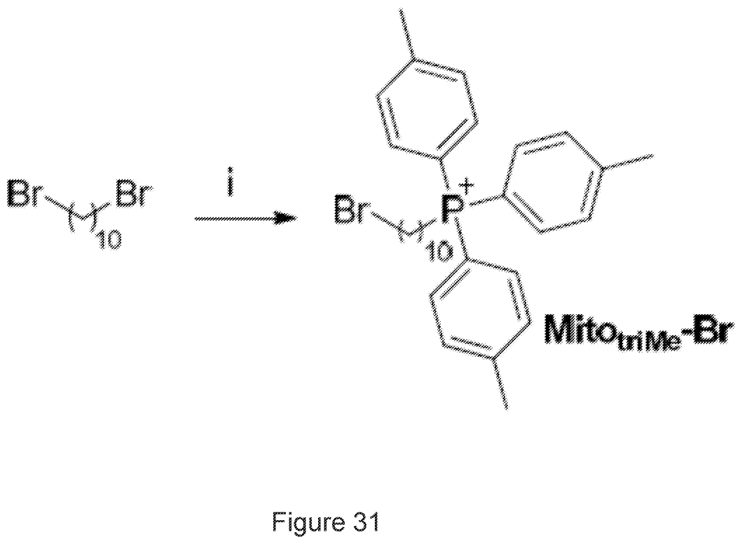

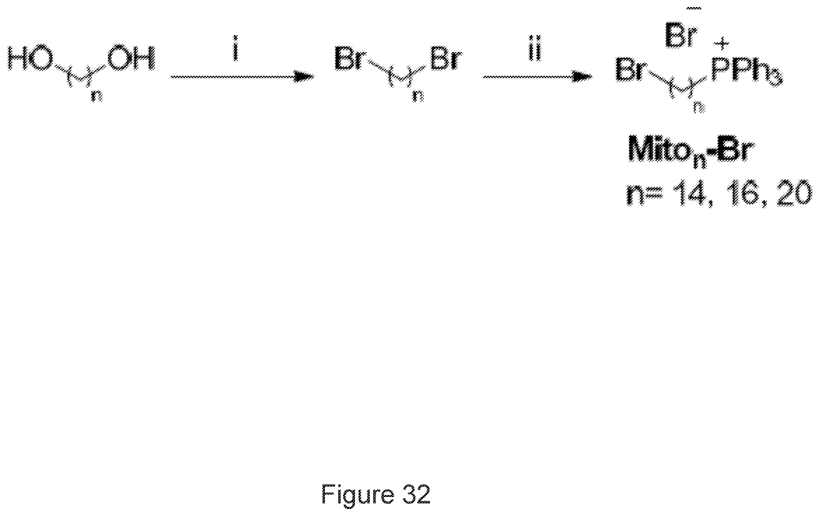

A scheme of the synthesis of HUs is presented in the supplementary materials (Schemes S1-S3). NMR spectra and related parameters are given in FIGS. 17-29.

Synthesis of 10-[(hydroxycarbamoyl)carbamoylamino]-decyltriphenylphosphonium trifluoroacetate (Mito₁₀-HU_{NHTPP+}). To a mixture of (10-aminodecyl)-triphenylphosphonium bromide (Cheng et al., 2016) (0.2 g, 0.37 mmol) and triethylamine (162 mL, 1.13 mmol) in dichloromethane (CH₂Cl₂), was added carbonyldiimidazole (CDI) (67 mg, 0.41 mmol) at 0° C. The mixture was stirred at room temperature for 4 hr. Then, HU (42 mg, 0.56 mmol) was added to the mixture with a few drops of DMF. The mixture was stirred at 50° C. for 12 hr. Then, the precipitated product was dissolved in water (H₂O) and purified on chromatography reverse phase to deliver the corresponding Mito₁₀-HU_{NHTPP+} (51 mg, 23% yield).

High-resolution mass spectrometry (HRMS) calculated for Mito-HU_{NHTPP+}C₃₀H₃₉N₃O₃P⁺ [M]⁺ 520.2724, found, 520.2723.

31P NMR (400.13 MHz, (CD₃)₂SO) d 24.05. ¹H NMR (400.13 MHz, (CD₃)₂SO), d 7.96-7.73 (15H, m), 7.43 (1H, bs),
6.31 (1H, bs), 3.51-3.34 (2H, m), 3.15-3.95 (2H, m), 1.60-1.35 (6H, m), 1.33-1.14 (10H, m). ¹³C NMR (75 MHz, (CD₃)₂SO) d 159.8, 155.9, 134.87, 134.84, 133.6, 133.5, 130.3, 130.1, 118.9, 118.1, 40.6, 29.9, 29.8, 29.6, 29.1, 28.8, 28.7, 28.6, 28.0, 26.1, 21.7 (d, J=3.7), 20.2 (d, J=49.9).

Synthesis of 10-[(carbamoylamino)oxy]-decyl-tris-p-tolylphosphonium trifluoroacetate (Mito_{triMe}-HU). A mixture of 1,10-dibromodecane (5 g, 16.6 mmol) and tris-p-tolylphosphine (1 g, 3.2 mmol) was stirred at 80° C. overnight. The reaction mixture was cooled. The residue was washed with diethyl ether (Et₂O). Purification by flash chromatography using pentane, Et₂O, and CH₂Cl₂/ethanol (EtOH; 9/1) as eluents successively delivered the corresponding (10-bromodecyl)-tris-p-tolylphosphonium (Mito_{triMe}-Br) (1.2 g, 60% yield).

HRMS calculated for Mito_{triMe}-Br C₃₁H₄₁BrP [M]⁺ 525.2108, found, 525.2113
31P (400.13 MHz, CDCl₃) d 23.17. 1H NMR (400.13 MHz, CDCl₃) d 7.70-7.61 (6H, m), 7.50-7.44 (6H, m), 3.63-3.50 (2H, m), 3.38 (2H, t, J=6.7 Hz), 2.47 (9H, s), 1.88-1.74 (3H, m), 1.64-1.52 (3H, m), 1.41-1.33 (2H, m), 1.28-1.17 (8H, m). ¹³C NMR (75 MHz, CDCl₃) d 146.14, 146.11, 133.4, 133.3, 131.0, 131.0, 115.6, 114.7, 34.0, 32.7, 30.3, 30.3, 29.1, 29.0, 28.9, 28.5, 27.9, 23.0 (J_{CP}=51.3), 22.5 (J_{CP}=4.4), 21.7.

To a mixture of HU (0.19 g, 2.4 mmol) and potassium carbonate (68 mg, 0.5 mmol) in DMF (4 mL) was added Mito_{triMe}-Br (0.3 g, 0.5 mmol). The mixture was stirred at 45° C. for 4 hr. Then, 100 mL of CH₂Cl₂ was added to the mixture. The organic layer was washed by 2 3 35 mL of H₂O. The organic layer was dried over sodium sulfate (Na₂SO₄), and the solvent was removed under reduced pressure. Then, Et₂O was added to the mixture to precipitate the compound. Purification by preparative high-performance liquid chromatography (HPLC) on C18 column (H₂O/CH₃CN containing 0.1% TFA as a gradient) was performed to deliver the corresponding Mito₁₀-HU_{triMe} (0.18 g, 57% yield).

HRMS calculated for Mito₁₀-HU_{triMe} C₃₂H₄₄N₂O₂P⁺ [M]⁺ 519.3135, found, 519.3134.

31P NMR (400.13 MHz, CD₃CN) d 22.58. ¹H NMR (400.13 MHz, CD₃CN), d 7.60-7.53 (12H, m), 3.78 (2H, t, J=6.7), 3.12-3.05 (2H, m), 2.5 (9H, s), 1.66-1.20 (16H, m). ¹³C NMR (75 MHz, CD₃CN) d 162.2, 160.7, 160.4, 160.0, 159.7, 147.1, 134.1, 133.9, 131.5, 131.4, 117.9, 116.4, 115.5, 77.0, 33.7, 33.6, 29.6, 29.4, 28.9, 28.4, 26.1, 22.5 (d, J=52.1), 22.6 (d, J=4.4), 21.4 (d, J=1.5).

Synthesis of 4-[(carbamoylamino)oxy]-butyltriphenylphosphonium trifluoroacetate (Mito₄-HU). To a mixture of HU (0.8 g, 10 mmol) and potassium carbonate (0.28 g, 2 mmol) in DMF (4 mL) was added (4-bromobutyl)-triphenylphosphonium bromide (1 g, 2 mmol). The mixture was stirred at 45° C. for 4 hr. 100 mL of CH₂Cl₂ was then added to the mixture. The organic layer was washed by 2 3 35 mL of H₂O. The aqueous layer is saturated in sodium chloride (NaCl) and extracted by 100 mL of chloroform (CHCl₃). The organic layer was dried over Na₂SO₄, and the solvent was removed under reduced pressure. Et₂O was then added to the mixture to precipitate the compound. Purification on C18 column (H₂O/CH₃CN containing 0.1% TFA as a gradient) was performed to deliver the corresponding Mito₄-HU (0.47 g, 48% yield).

HRMS calculated for Mito₄-HU C₂₃H₂₆N₂O₂P⁺ [M]⁺ 393.1726, found, 393.1728.

31P NMR (400.13 MHz, (CD₃)₂SO) d 23.93. ¹H NMR (400.13 MHz, (CD₃)₂SO), d 7.97-7.69 (15H, m), 6.31 (2H, s), 3.78-3.68 (4H, m), 1.86-1.76 (2H, m), 1.68-1.57 (2H, m). ¹³C NMR (75 MHz, (CD₃)₂SO) d 160.9, 135.0, 134.8, 133.8, 133.7, 133.6, 133.5, 130.4, 130.3, 130.2, 119.0, 118.2, 74.1, 28.1 (d, J=16.9), 20.2 (d, J=49.9), 18.8 (d, J=2.9).

Synthesis of 10-[(carbamoylamino)oxy]-decyltriphenylphosphonium trifluoroacetate (Mito₁₀-HU). To a mixture of HU (0.4 g, 5.0 mmol) and potassium carbonate (0.13 g, 0.9 mmol) in DMF (4 mL) was added (10-bromodecyl)-triphenylphosphonium bromide (Pan et al., 2018) (0.5 g, 0.9 mmol). The mixture was stirred at 45° C. for 4 hr. 100 mL of CH₂Cl₂ was then added to the mixture. The organic layer was washed by 2 3 35 mL of H₂O. The organic layer was dried over Na₂SO₄ and the solvent was removed under reduced pressure. Et₂O was then added to the mixture to precipitate the compound. Purification by preparative HPLC on C18 column (H₂O/CH₃CN containing 0.1% TFA as a gradient) was performed to deliver the corresponding Mito₁₀-HU (0.22 g, 44% yield).

HRMS calculated for Mito₁₀-HU C₂₉H₃₈N₂O₂P⁺ [M]⁺ 477.2665, found, 477.2667.

31P NMR (400.13 MHz, CDCl₃) d 24.06. ¹H NMR (400.13 MHz, CDCl₃), d 7.86-7.60 (15H, m), 3.81 (2H, t, J=6.5), 3.35-3.19 (2H, m), 1.70-1.40 (6H, m), 1.36-1.11 (10H, m). ¹³C NMR (75 MHz, CDCl₃) d 160.7, 160.3, 135.3, 135.2, 133.3, 133.2, 130.7, 130.5, 118.5, 117.4, 76.8, 30.4, 30.2, 29.0, 28.9, 28.8, 28.6, 27.8, 25.6, 22.4 (d, J=50.1), 22.4 (d, J=4.4).

Synthesis of 12-[(carbamoylamino)oxy]-dodecyltriphenylphosphonium trifluoroacetate (Mito₁₂-HU). To a mixture of HU (0.35 g, 4.60 mmol) and potassium carbonate (0.13 g, 0.9 mmol) in DMF (3 mL) was added (12-bromododecyl)-triphenylphosphonium bromide (Cheng et al., 2020a) (0.5 g, 0.85 mmol). The mixture was stirred at 45° C. for 4 hr. 100 mL of CH₂Cl₂ was then added to the mixture. The organic layer was washed by 2 3 35 mL of H₂O. The organic layer was dried over Na₂SO₄ and the solvent was removed under reduced pressure. Et₂O was then added to the mixture to precipitate the compound. Purification by low-pressure chromatography on C18 column (H₂O/CH₃CN containing 0.1% TFA as a gradient) was performed to deliver the corresponding Mito₁₂-HU (0.2 g, 38% yield).

HRMS calculated for Mito12-HU C₃₁H₄₂N₂O₂P⁺ [M]⁺505.2978, found, 505.2980.

31P NMR (400.13 MHz, (CD₃)₂SO) d 24.06. ¹H NMR (400.13 MHz, (CD₃)₂SO), d 7.91-7.73 (15H, m), 3.65 (2H, t, J=6.9), 3.61-3.49 (2H, m), 1.59-1.39 (6H, m), 1.33-1.14 (14H, m). ¹³C NMR (75 MHz, (CD₃)₂SO) d 160.9, 134.9, 134.8, 133.7, 133.5, 133.4, 133.3, 130.3, 130.2, 130.1, 119.0, 118.2, 75.4, 28.9, 28.1, 28.7, 27.6, 21.7 (d, J=4.4), 20.2 (d, J=49.9).

Synthesis of 14-[(carbamoylamino)oxy]-tetradecyltriphenylphosphonium trifluoroacetate (Mito₁₄-HU). Aqueous hydrogen bromide (48%, 70 mL, 4.2 mmol) was added dropwise to acetic anhydride (120 mL, 1.27 mol) at 0° C. To this solution, 14-hydroxytetradecanol (5 g, 0.02 mol) was added and the mixture was brought to reflux for 24 hr. Then, the dihalide was extracted with pentane and washed with an excess of H₂O (5 3 50 mL) and sodium bicarbonate (50 mL). The organic layer was dried over Na₂SO₄. The solvent was removed under reduced pressure. 1,14-dibromotetradecane was obtained in quantitative yield.

1,14-Dibromotetradecane: ¹H NMR (400.13 MHz, CDCl₃) d 3.41 (t, 4H, J=6.9), 1.86 (quint., 4H, J=6.9, 14.1), 1.49-1.37 (4H, m), 1.36-1.24 (16H, m).

A mixture of 1,14-dibromotetradecane (6.7 g, 0.018 mol) and triphenylphosphine (1 g, 3.8 mmol) was stirred at 90° C. for 6 hr. The resulting mixture was washed with Et₂O. Purification by flash chromatography using pentane, Et₂O, and CH₂Cl₂/EtOH (9/1) successively delivered the corresponding Mito₁₄-Br (1.9 g, 80% yield).

(14-Bromotetradecyl)-triphenylphosphonium (Mito₁₄-Br):³¹P NMR (400.13 MHz, CDCl₃) d 24.54. ¹H NMR (400.13 MHz, CDCl₃), d 7.94-7.63 (15H, m), 3.90-3.78 (2H, m), 3.40 (t, 2H, J=6.9), 1.85 (quint., 2H, J=7.0, 14.0), 1.66-1.57 (4H, m), 1.46-1.37 (2H, m), 1.30-1.14 (16H, m). ¹³C NMR (75 MHz, CDCl₃) d 134.9, 134.8, 133.5, 133.4, 130.4, 130.3, 118.5, 117.7, 33.4, 32.6, 30.3, 30.1, 29.3, 29.2, 29.1, 28.9, 28.5, 27.9, 22.9, 22.5, 22.4.

To a mixture of HU (0.4 g, 5.2 mmol) and potassium carbonate (0.13 g, 0.9 mmol) in DMF (3 mL) was added (14-bromotetradecyl)-triphenylphosphonium bromide, (0.5 g, 0.85 mmol). The mixture was stirred at 45° C. for 4 hr. 100 mL of CH₂Cl₂ was then added to the mixture. The organic layer was washed by 2 3 35 mL of H₂O. The organic layer was dried over Na₂SO₄ and the solvent was removed under reduced pressure. Et₂O was then added to the mixture to precipitate the compound. Purification by low-pressure chromatography on C18 column (H₂O/CH₃CN containing 0.1% TFA as a gradient) was performed to deliver the corresponding Mito₁₄-HU (0.23 g, 46% yield).

HRMS calculated for Mito₁₄-HU C₃₃H₄₆N₂O₂P⁺ [M]⁺ 533.3291, found, 533.3290.

31P NMR (400.13 MHz, CD₃CN) d 23.63. ¹H NMR (400.13 MHz, CD₃CN), d 7.92-7.63 (15H, m), 5.71-5.23 (2H, m), 3.76 (2H, t, J=6.9), 3.21-3.10 (2H, m), 1.68-1.56 (4H, m), 1.52-1.42 (2H, m), 1.38-1.18 (18H, m). ¹³C NMR (75 MHz, CD₃CN) d 136.2, 136.1, 134.7, 133.6, 131.4, 131.3, 119.9, 119.1, 118.4, 77.4, 31.2, 31.1, 30.4, 30.33, 30.32, 30.2, 29.9, 29.4, 28.9, 26.7, 23.0 (d, J=4.4), 22.7 (d, J=51.3).

Synthesis of 16-[(carbamoylamino)oxy]-hexadecyltriphenylphosphonium trifluoroacetate (Mito₁₆-HU). Aqueous hydrogen bromide (48%, 70 mL, 4.2 mmol) was added dropwise to acetic anhydride (120 mL, 1.27 mol) at 0° C. To this solution, 16-hydroxyhexadecanol (5 g, 0.02 mol) was added and the mixture was brought to reflux for 24 hr. The dihalide was then extracted with pentane and washed with excess of H₂O (5 3 50 mL) and sodium bicarbonate (50 mL). The organic layer was dried over Na₂SO₄. The solvent was removed under reduced pressure. 1,16-dibromohexadecane was obtained in quantitative yield.

1,16-Dibromohexadecane: ¹H NMR (400.13 MHz, CDCl₃) d 3.41 (4H, t, J=6.8), 1.86 (4H, quint., J=7.0, 14.0), 1.48-1.38 (4H, m), 1.35-1.25 (20H, m).

A mixture of 1,16-dibromohexadecane (6 g, 15 mmol) and triphenylphosphine (1 g, 3.8 mmol) was stirred at 90° C. for 6 hr. The resulting mixture was washed with Et₂O. Purification by flash chromatography using pentane, Et₂O, and CH₂Cl₂/EtOH (9/1) as eluents successively delivered the corresponding (16-Bromohex-adecyl)-triphenylphosphonium, Mito₁₆-Br (1.3 g, 53% yield).

HRMS calculated for Mito₁₆-Br C₃₄H₄₇BrP⁺ [M]⁺ 565.2593, found, 565.2593.

(16-Bromohexadecyl)-triphenylphosphonium (Mito₁₆-Br): ³¹P NMR (400.13 MHz, CDCl₃d 24.29. ¹H NMR (400.13 MHz, CDCl₃), d 7.87-7.64 (15H, m), 3.76-3.65 (2H, m), 3.37 (2H, t, J=6.9), 1.86-1.75 (2H, m), 1.65-1.54 (4H, m), 1.43-1.31 (2H, m), 1.32-1.12 (20H, m). ¹³C NMR (75 MHz, CDCl₃) d 134.94, 134.91, 133.6, 133.5, 130.4, 130.3, 118.7, 117.8, 33.9, 32.7, 30.4, 30.2, 29.4, 29.3, 29.2, 29.0, 28.6, 28.0, 22.8 (d, J=49.9), 22.5 (d, J=4.4).

To a mixture of HU (0.2 g, 2.6 mmol) and potassium carbonate (66 mg, 0.48 mmol) in DMF (3 mL) was added (16-bromohexadecyl)-triphenylphosphonium bromide (0.31 g, 0.48 mmol). The mixture was stirred at 45° C. for 4 hr. 100 mL of CH₂Cl₂ was then added to the mixture. The organic layer was washed by 2 3 35 mL of H₂O. The organic layer was dried over Na₂SO₄ and the solvent was removed under reduced pressure. Et₂O was then added to the mixture to precipitate the compound. Purification by low-pressure chromatography on C18 column (H₂O/CH₃CN containing 0.1% TFA as a gradient) was performed to deliver the corresponding Mito₁₆-HU (0.27 g, 82% yield).

HRMS calculated for Mito₁₆-HU C₃₅H₅₀N₂O₂P⁺ [M]⁺ 561.3604, found, 561.3611.

31P NMR (400.13 MHz, CD₃CN) d 23.67. ¹H NMR (400.13 MHz, CD₃CN), d 7.92-7.82 (3H, m), 7.75-7.68 (12H, m), 5.75-5.35 (2H, m), 3.75 (2H, t, J=6.6), 3.23-3.14 (2H, m), 1.68-1.55 (4H, m), 1.53-1.44 (2H, m), 1.36-1.19 (22H, m). ¹³C NMR (75 MHz, CD₃CN) d 61.7, 160.7, 160.0, 159.6, 136.2, 136.1, 134.8, 133.7, 131.4, 131.3, 119.9, 119.1, 118.4, 77.3, 31.2, 31.1, 30.42, 30.40, 30.37, 30.35, 30.34, 30.2, 29.9, 29.4, 28.9, 26.7, 23.0 (d, J=4.4), 22.7 (d, J=51.4).

Synthesis of 20-[(carbamoylamino)oxy]-eicosanyltriphenylphosphonium trifluoroacetate (Mito₂₀-HU). 20-Hydroxyeicosanol was obtained by adapting the procedures from Chanda et al. (Chanda and Ramakrishnan, 2015).

Aqueous hydrobromic acid (48%, 70 mL, 4.2 mmol) was added dropwise to acetic anhydride (120 mL, 1.27 mol) at 0° C. To this solution, 20-hydroxyeicosanol (6 g, 0.02 mol) was added and the mixture was brought to reflux for 24 hr. Then, the dihalide was extracted with pentane (500 mL) and washed with excess of H₂O (5 3 100 mL) and saturated solution of sodium bicarbonate (2 3 100 mL). The organic layer was dried over sodium sulfate. The solvent was removed under reduced pressure. 1,20-dibromoeicosane was obtained in 89% yield (7.5 g).

A mixture of 1,20-dibromoeicosane (6.6 g, 15 mmol) and triphenylphosphine (1 g, 3.8 mmol) was stirred at 90° C. for 12 hr. The resulting mixture was washed with Et₂O. Purification by flash chromatography using pentane, Et₂O, and CH₂Cl₂/EtOH (9/1) as eluents successively delivered the corresponding (20-bromoei-cosanyl)-triphenylphosphonium bromide, Mito-Br-C₂₀ (1.5 g, 56% yield).

ESI-MS for Mito-Br-C₂₀ C₃₈H₅₅BrP⁺ [M]⁺ 624.4.

To a mixture of HU (0.35 g, 4.6 mmol) and potassium carbonate (0.13 g, 0.94 mmol) in DMF (3 mL) was added (20-bromoeicosanyl)-triphenylphosphonium bromide, (0.50 g, 0.71 mmol). The mixture was stirred at 45° C. for 4 hr. Then, 100 mL of CH₂Cl₂ was added to the mixture. The organic layer was washed by 2 3 35 mL of H₂O. The organic layer was dried over Na₂SO₄ and the solvent was removed under reduced pressure. Then, sodium sulfate was added to the mixture to precipitate the compound. Purification by C18 column (using H₂O/MeCN [9/1 to 100%] containing 0.1% TFA) was performed to deliver the corresponding Mito₂₀-HU (0.26 g, 50% yield).

HRMS calculated for Mito₂₀-HU C₃₉H₅₈N₂O₂P⁺ [M]⁺ 617.4230, found, 617.4231. 31P NMR (400.13 MHz, CDCl₃) d 23.61. ¹H NMR (400.13 MHz, CDCl₃), d 7.83-7.76 (3H, m), 7.72-7.65 (12H, m), 3.81 (2H, t, J=6.7), 3.37-3.26 (2H, m), 1.66-1.56 (4H, m), 1.56-1.48 (2H, m), 1.30-1.15 (30H, m). ¹³C NMR (75 MHz, CD₃CN) d 162.4, 160.9, 160.5, 136.2, 136.1, 134.7, 133.6, 131.4, 131.3, 119.9, 119.1, 118.5, 77.4, 31.2, 31.0, 30.5, 30.4, 30.39, 30.35, 30.24, 30.23, 29.9, 29.4, 28.9, 26.7, 23.0 (d, J=4.4), 22.7 (d, J=50.6).

Synthesis of decyloxyurea(Decyl-HU). To a mixture of HU (1.7 g, 22.3 mmol) and potassium carbonate (0.62 g, 4.5 mmol) in DMF (4 mL) was added bromodecane (1 g, 4.5 mmol). The mixture was stirred at 45° C. for 4 hr. 100 mL of CH₂Cl₂ was then added to the mixture. The organic layer was washed by 2 3 35 mL of H₂O. The organic layer was dried over Na₂SO₄ and the solvent removed under reduced pressure. Et₂O was then added to the mixture to precipitate the corresponding to decyl-HU (0.3 g, 31% yield). HRMS calculated for decyl-HU C₁₁H₂₄N₂O₂ [MH]⁺ 217.1911, found, 217.1911. ¹H NMR (400.13 MHz, (CD₃)₂SO), d¹H NMR (400.13 MHz, (CD₃)₂SO), d6.23 (2H, s), 3.64 (2H, t, J=6.5), 1.60-1.46 (2H, m), 1.34-1.17 (14H, m), 0.85 (3H, t, 6.7). ¹³C NMR (75 MHz, CDCl₃) d 0.8, 75.4, 31.3, 28.95, 28.90, 28.8, 27.6, 25.3, 22.1, 13.9, 13.8.

### Cell Proliferation Measurements

The IncuCyte Live-Cell Analysis system (Essen Bioscience Inc., Ann Arbor, MI) was used to monitor cell proliferation (Cheng et al., 2013, 2016; Boyle et al., 2018). As shown in previous publications (Cheng et al., 2013, 2016), this imaging system is probe-free and noninvasive, and enables continuous monitoring of cell confluence over several days. The increase in the percentage of cell confluence was used as a surrogate marker of cell proliferation. In a 96-well plate, cells were plated at 1,000 cells per well in triplicates and left to adhere overnight. Cells were then treated with HU and Mito-HUs, and the cell confluency was recorded over several days in the IncuCyte Live-Cell Analysis system.

### Cytotoxicity Assay

To determine the cytotoxicity of Mito-HUs, we used the SYTOX Green-based assay (Cheng et al., 2012). HCT116 cells were treated for up to 48 hr, and dead cells were monitored in real time in the presence of 200 nM SYTOX Green (Invitrogen, Carlsbad, CA) under an atmosphere of 5% CO₂:95% air at 37° C. Cells were then permeabilized with digitonin (120 mM) in the presence of SYTOX Green to determine the total cell number.

### Mitochondrial Oxygen Consumption Measurements

Mitochondrial oxygen consumption was measured using the Seahorse XF-96 Extracellular Flux Analyzer (Agilent, North Billerica, MA) (Cheng et al., 2013, 2016; Boyle et al., 2018; Weinberg and Chandel, 2015). After cells were treated with HU or Mito-HUs for 24 hr, the OCR-based assessment of mitochondrial complex activities was carried out on acutely permeabilized cells in the presence of different mitochondrial substrates, i.e., pyruvate/malate for complex I and duroquinol for complex III (Cheng et al., 2016, 2019; Salabei et al., 2014; Wheaton et al., 2014). Rotenone, malonate, and antimycin A (Sigma-Aldrich, St. Louis, MO) were used as specific inhibitors of mitochondrial complexes I, II, and III, respectively. Briefly, cells that were intact after treatments were immediately permeabilized using the Seahorse XF Plasma Membrane Permeabilizer (Agilent). The mitochondrial complex I-induced OCR was assayed in mannitol and sucrose buffer (Salabei et al., 2014) containing 10 mM pyruvate and 1.5 mM malate (substrates for complex I) and 10 mM malonate (which inhibits complex II activities). The mitochondrial complex III-driven OCR was assayed in a mannitol and sucrose buffer containing 0.5 mM duroquinol (substrate for complex III) as well as 1 mM rotenone and 10 mM malonate (which inhibit both complex I and II activities). The IC₅₀ values were determined as previously reported (Cheng et al., 2019).

### Immunoregulatory Measurements

To differentiate CD4⁺ T cells into a T_{reg} phenotype, splenocytes from SMARTA triple-reporter mice were processed and the red blood cells were lysed using an ACK (ammonium-chloride-potassium) lysis buffer. The cells were then activated with 1 mg/mL GP₆₁₋₈₀ peptide (GenScript, Piscataway, NJ) and 5 ng/mL TGF-b1 (Shenandoah Biotechnology, Inc., Warwick, PA). After one day of initial skewing, 100 mg/mL IL-2 along with HU or Mito-HUs (n=4-20) of varying concentrations were added to the culture. Cells were cultured for six days and split once cells reached confluency; cells were replenished with IL-2 and compound (i.e., MitoHUs) accordingly. After six days in culture, cells were stained to assess the viability and phenotypic analysis via flow cytometry. LIVE/DEAD fixable violet or aqua dead cell stain (Invitrogen) was used to assess cell viability. Only live cells were gated out for further functional analysis by using a fixable LIVE/DEAD stain. First, total population of cells was gated (FSC-A vs SSC-A). Then, single cells were gated (SSC-W vs SSC-H). Within the single cell population, we gated on the live CD4 T cells using a fixable Live/Dead stain (CD4 vs DEAD). Within the live CD4 T cells, we gated on CD25⁺FOXP3⁺ T_{regs} (FOXP3 vs CD25), as well as IFNg-YFP⁺ CD4 T cells (IFNg-YFP vs CD4).

The following antibodies were used for flow cytometry staining: APC-Cy7 anti-mouse CD4 (clone: GK1.5; BioLegend, San Diego, CA), APC anti-mouse CD25 (clone: PC61; BioLegend), and PE anti-mouse FOXP3 (clone: FJK-165; eBioscience, San Diego, CA). Flow cytometry data were acquired using a BD Celesta flow cytometer (BD Biosciences, San Jose, CA) flow cytometer and analyzed using FlowJo (Treestar, Inc., Ashland, OR) (FlowJo Software, 2019).

MDSCs were generated from bone marrow cells isolated from C57b1/6 mice and cultured at a density of 1 3 10⁶ cells/ml in RPMI with 10% fetal bovine serum and 25 ng/ml recombinant mouse GM-CSF (Shenandoah Biotechnology, Inc.) and 25 ng/ml recombinant mouse IL-6 (Shenandoah Biotechnology, Inc.) for three days. Then, cells were re-cultured in fresh media with inhibitors for an additional three days. Cells were scraped and examined by flow cytometry using the following markers: FITC-anti Ly6C (Biolegend, San Diego, CA), PE-Cy7 anti F4/80 (Biolegend), APC-Cy7 anti Ly6G (Biolegend), and Pacific Blue anti-CD11b (Biolegend). A gating strategy similar to that employed for the T cells was used in all MDSC differentiation assays, which were tested only on live cells after treatments. Flow cytometry data were acquired using a BD Celesta flow cytometer (BD Biosciences), and data were analyzed using FlowJo software (Treestar, Inc.).

### Quantification and Statistical Analysis

Comparisons between the control group and treatment group were made using an unpaired Student's ttest analysis. p values of less than 0.05 were considered to be statistically significant. All values provided represent mean G standard deviation. The numbers of replicates per treatment group are shown as n. IC₅₀ values and fitting curves were calculated using OriginPro 2016 (OriginLab Corporation, Northampton, MA).

### REFERENCES

American Association for Cancer Research (2019). Drugging OXPHOS dependency in cancer. Cancer Discov. 9, Of 10.

Ashton, T. M., Mckenna, W. G., Kunz-Schughart, L. A., and Higgins, G. S. (2018). Oxidative phosphorylation as an emerging target in cancer therapy. Clin. Cancer Res. 24, 2482-2490.

Asin-Cayuela, J., Manas, A.-R. B., James, A. M., Smith, R. A. J., and Murphy, M. P. (2004). Fine-tuning the hydrophobicity of a mitochondria-targeted antioxidant. FEB S Lett. 571, 9-16.

Benjamin, D., Robay, D., Hindupur, S. K., Pohlmann, J., Colombi, M., El-Shemerly, M. Y., Maira, S. M., Moroni, C., Lane, H. A., and Hall, M. N. (2018). Dual inhibition of the lactate transporters MCT1 and MCT4 is synthetic lethal with metformin due to NAD+ depletion in cancer cells. Cell Rep. 25, 3047-3058.e4.

Boyle, K. A., Van Wickle, J., Hill, R. B., Marchese, A., Kalyanaraman, B., and Dwinell, M. B. (2018). Mitochondria-targeted drugs stimulate mitophagy and abrogate colon cancer cell proliferation. J. Biol. Chem. 293, 14891-14904.

Chanda, S., and Ramakrishnan, S. (2015). Poly(alkylene itaconate)s—an interesting class of polyesters with periodically located exo-chain double bonds susceptible to Michael addition. Polym. Chem. 6, 2108-2114.

Chang, M. C. Y., Yee, C. S., Stubbe, J., and Nocera, D. G. (2004). Turning on ribonucleotide reductase by light-initiated amino acid radical generation. Proc. Natl. Acad. Sci. U S A 101, 6882.

Cheng, G., Hardy, M., Topchyan, P., Zander, R., Volberding, P., Cui, W., and Kalyanaraman, B. (2020a). Potent inhibition of tumour cell proliferation and immunoregulatory function by mitochondria-targeted atovaquone. Sci. Rep. 10, 17872.

Cheng, G., Hardy, M., Zielonka, J., Weh, K. M., Zielonka, M., Boyle, K., Abu Eid, M., Mcallister, D., Bennett, B., Kresty, L. A., et al. (2020b). Mitochondria-targeted magnolol inhibits OXPHOS, proliferation, and tumor growth via modulation of energetics and autophagy in melanoma cells. Cancer Res. Treat.Commun. 25, 100210.

Cheng, G., Zhang, Q., Pan, J., Lee, Y., Ouari, O., Hardy, M., Zielonka, M., Myers, C. R., Zielonka, J., Weh, K., et al. (2019). Targeting lonidamine to mitochondria mitigates lung tumorigenesis and brain metastasis. Nat. Commun. 10, 2205.

Cheng, G., Zielonka, J., Dranka, B. P., Mcallister, D., Mackinnon, A. C., Jr., Joseph, J., and Kalyanaraman, B. (2012). Mitochondria-targeted drugs synergize with 2-deoxyglucose to trigger breast cancer cell death. Cancer Res. 72, 2634-2644.

Cheng, G., Zielonka, J., Mcallister, D. M., Mackinnon, A. C., Jr., Joseph, J., Dwinell, M. B., and Kalyanaraman, B. (2013). Mitochondria-targeted vitamin E analogs inhibit breast cancer cell energy metabolism and promote cell death. BMC Cancer 13, 285.

Cheng, G., Zielonka, J., Ouari, O., Lopez, M., Mcallister, D., Boyle, K., Barrios, C. S., Weber, J. J., Johnson, B. D., Hardy, M., et al. (2016). Mitochondria-targeted analogues of metformin exhibit enhanced antiproliferative and radiosensitizing effects in pancreatic cancer cells. Cancer Res. 76, 3904-3915.

Corzo, C. A., Cotter, M. J., Cheng, P., Cheng, F., Kusmartsev, S., Sotomayor, E., Padhya, T., Mccaffrey, T. V., Mccaffrey, J. C., and Gabrilovich, D. I. (2009). Mechanism regulating reactive oxygen species in tumor-induced myeloid-derived suppressor cells. J. Immunol. 182, 5693— 5701.

Das, M., Alzaid, F., and Bayry, J. (2019). Regulatory T cells under the mercy of mitochondria. Cell Metab. 29, 243-245.

Feng, S., Cheng, X., Zhang, L., Lu, X., Chaudhary, S., Teng, R., Frederickson, C., Champion, M. M., Zhao, R., Cheng, L., et al. (2018). Myeloid-derived suppressor cells inhibit T cell activation through nitrating LCK in mouse cancers. Proc. Natl. Acad. Sci. U S A 115, 10094-10099.

Fiorillo, M., Lamb, R., Tanowitz, H. B., Mutti, L., Krstic-Demonacos, M., Cappello, A. R., Martinez-Outschoorn, U. E., Sotgia, F., and Lisanti, M. P. (2016). Repurposing atovaquone: targeting mitochondrial complex III and OXPHOS to eradicate cancer stem cells. Oncotarget 7, 34084-34099.

Fischer, G. M., Jalali, A., Kircher, D. A., Lee, W. C., Mcquade, J. L., Haydu, L. E., Joon, A. Y., Reuben, A., De Macedo, M. P., Carapeto, F. C. L., et al. (2019). Molecular profiling reveals unique immune and metabolic features of melanoma brain metastases. Cancer Discov. 9, 628-645.

FlowJo Software (2019). FlowJo Software, Tenth Edition (Becton, Dickinson and Company).

Groth, C., Hu, X., Weber, R., Fleming, V., Altevogt, P., Utikal, J., and Umansky, V. (2019). Immunosuppression mediated by myeloid- derived suppressor cells (MDSCs) during tumour progression. Br. J. Cancer 120, 16-25.

Grund, F. M., Armitage, J. O., and Burns, C. P. (1977). Hydroxyurea in the prevention of the effects of leukostasis in acute leukemia. Arch. Intern. Med. 137, 1246-1247.

Hossain, F., Al-Khami, A. A., Wyczechowska, D., Hernandez, C., Zheng, L., Reiss, K., Valle, L. D., Trillo-Tinoco, J., Maj, T., Zou, W., et al. (2015). Inhibition of fatty acid oxidation modulates immunosuppressive functions of myeloid-derived suppressor cells and enhances cancer therapies. Cancer Immunol. Res. 3, 1236-1247.

Kawano, Y., Moschetta, M., Manier, S., Glavey, S., Go\ddot{ } rgu\ddot{ } n, G. T., Roccaro, A. M., Anderson, K. C., and Ghobrial, I. M. (2015). Targeting the bone marrow microenvironment in multiple myeloma. Immunol. Rev. 263, 160-172.

Klopman, G., Li, J.-Y., Wang, S., and Dimayuga, M. (1994). Computer automated log P calculations based on an extended group contribution approach. J. Chem. Inf. Comput. Sci. 34, 752-781.

Koc, A., Wheeler, L. J., Mathews, C. K., and Merrill, G. F. (2004). Hydroxyurea arrests DNA replication by a mechanism that preserves basal dNTP pools. J. Biol. Chem. 279,223-230.

Langley, M., Ghosh, A., Charli, A., Sarkar, S., Ay, M., Luo, J., Zielonka, J., Brenza, T., Bennett, B., Jin, H., et al. (2017). Mito-apocynin prevents mitochondrial dysfunction, microglial activation, oxidative damage, and progressive neurodegeneration in MitoPark transgenic mice. Antioxid. Redox Signal. 27, 1048-1066.

Lassmann, G., Thelander, L., and Gra\ddot{ } slund, A. (1992). EPR stopped-flow studies of the reaction of the tyrosyl radical of protein R2 from ribonucleotide reductase with hydroxyurea. Biochem.Biophys. Res. Commun. 188, 879-887.

Law, A. M. K., Valdes-Mora, F., and Gallego-Ortega, D. (2020). Myeloid-derived suppressor cells as a therapeutic target for cancer. Cells 9,561.

Leyden, D., Ahmed, N., and Hassan, H. T. (2000). Hydroxyurea and trimidox enhance the radiation effect in human pancreatic adenocarcinoma cells. Anticancer Res. 20, 133-138.

Madaan, K., Kaushik, D., and Verma, T. (2012). Hydroxyurea: a key player in cancer chemotherapy. Expert Rev. Anticancer Ther. 12, 19-29.

Mai, X., Lu, X., Xia, H., Cao, Y., Liao, Y., and Lv, X. (2010). Synthesis, antitumor evaluation and crystal structure of hydroxyurea derivatives. Chem. Pharm.Bull. 58, 94-97.

Mastio, J., Condamine, T., Dominguez, G., Kossenkov, A. V., Donthireddy, L., Veglia, F., Lin, C., Wang, F., Fu, S., Zhou, J., et al. (2019). Identification of monocyte-like precursors of granulocytes in cancer as a mechanism for accumulation of PMN-MDSCs. J. Exp. Med. 216, 2150-2169.

Nagaraj, S., Youn, J. I., and Gabrilovich, D. I. (2013). Reciprocal relationship between myeloid-derived suppressor cells and T cells. J. Immunol. 191, 17-23.

Oo, Z. Y., Proctor, M., Stevenson, A. J., Nazareth, D., Fernando, M., Daignault, S. M., Lanagan, C., Walpole, S., Bonazzi, V., S˜kalamera, D., et al. (2019). Combined use of subclinical hydroxyurea and CHK1 inhibitor effectively controls melanoma and lung cancer progression, with reduced normal tissue toxicity compared to gemcitabine. Mol. Oncol. 13, 1503-1518.

Oxenius, A., Bachmann, M. F., Zinkernagel, R. M., and Hengartner, H. (1998). Virus-specific MHC-class II-restricted TCR-transgenic mice: effects on humoral and cellular immune responses after viral infection. Eur. J. Immunol. 28, 390-400.

Pan, J., Lee, Y., Cheng, G., Zielonka, J., Zhang, Q., Bajzikova, M., Xiong, D., Tsaih, S.-W., Hardy, M., Flister, M., et al. (2018). Mitochondria-targeted honokiol confers a striking inhibitory effect on lung cancer via inhibiting complex I activity. iScience 3, 192-207.

Reinhardt, R. L., Liang, H.-E., and Locksley, R. M. (2009). Cytokine-secreting follicular T cells shape the antibody repertoire. Nat. Immunol. 10, 385-393.

Ren, S., Wang, R., Komatsu, K., Bonaz-Krause, P., Zyrianov, Y., Mckenna, C. E., Csipke, C., Tokes, Z. A., and Lien, E. J. (2002). Synthesis, biological evaluation, and quantitative Structure Activity relationship analysis of new schiff bases of hydroxysemicarbazide as potential antitumor agents. J. Med. Chem. 45, 410-419.

Salabei, J. K., Gibb, A. A., and Hill, B. G. (2014). Comprehensive measurement of respiratory activity in permeabilized cells using extracellular flux analysis. Nat. Protoc. 9, 421-438.

Saleh, R., and Elkord, E. (2020). FoxP3(+) T regulatory cells in cancer: prognostic biomarkers and therapeutic targets. Cancer Lett. 490, 174-185.

Sawant, A., Schafer, C. C., Jin, T. H., Zmijewski, J., Tse, H. M., Roth, J., Sun, Z., Siegal, G. P., Thannickal, V. J., Grant, S. C., et al. (2013). Enhancement of antitumor immunity in lung cancer by targeting myeloid-derived suppressor cell pathways. Cancer Res. 73, 6609-6620.

Shulman, Z., Gitlin, A. D., Weinstein, J. S., Lainez, B., Esplugues, E., Flavell, R. A., Craft, J. E., and Nussenzweig, M. C. (2014). Dynamic signaling by T follicular helper cells during germinal center B cell selection. Science 345, 1058-1062.

Singh, A., and Xu, Y. J. (2016). The cell killing mechanisms of hydroxyurea. Genes (Basel) 7, 99.

Solito, S., Falisi, E., Diaz-Montero, C. M., Doni, A., Pinton, L., Rosato, A., Francescato, S., Basso, G., Zanovello, P., Onicescu, G., et al. (2011). A human promyelocytic-like population is responsible for the immune suppression mediated by myeloid-derived suppressor cells. Blood 118, 2254-2265.

Spivak, J. L., and Hasselbalch, H. (2011). Hydroxycarbamide: a user's guide for chronic myeloproliferative disorders. Expert Rev. Anticancer Ther. 11, 403-414.

Teng, J., Hejazi, S., Hiddingh, L., Carvalho, L., De Gooijer, M. C., Wakimoto, H., Barazas, M., Tannous, M., Chi, A. S., Noske, D. P., et al. (2018). Recycling drug screen repurposes hydroxyurea as a sensitizer of glioblastomas to temozolomide targeting de novo DNA synthesis, irrespective of molecular subtype. Neuro Oncol. 20, 642-654.

Trovato, R., Fiore, A., Sartori, S., Cane', S., Giugno, R., Cascione, L., Paiella, S., Salvia, R., De Sanctis, F., Poffe, O., et al. (2019). Immunosuppression by monocytic myeloid-derived suppressor cells in patients with pancreatic ductal carcinoma is orchestrated by STAT3. J. Immunother.Cancer 7, 255.

Veglia, F., Perego, M., and Gabrilovich, D. (2018). Myeloid-derived suppressor cells coming of age. Nat. Immunol. 19, 108-119.

Viswanadhan, V. N., Ghose, A. K., Revankar, G. R., and Robins, R. K. (1989). Atomic physicochemical parameters for three dimensional structure directed quantitative structure-activity relationships. 4. Additional parameters for hydrophobic and dispersive interactions and their application for an automated superposition of certain naturally occurring nucleoside antibiotics. J. Chem. Inf. Comput. Sci. 29, 163-172.

Weinberg, S. E., and Chandel, N. S. (2015). Targeting mitochondria metabolism for cancer therapy. Nat. Chem. Biol. 11, 9-15.

Weinberg, S. E., Singer, B. D., Steinert, E. M., Martinez, C. A., Mehta, M. M., Martinez-Reyes, I., Gao, P., Helmin, K. A., Abdala-Valencia, H., Sena, L. A., et al. (2019). Mitochondrial complex III is essential for suppressive function of regulatory T cells. Nature 565, 495-499.

Weinstein, J. S., Herman, E. I., Lainez, B., Licona-Limo', P., Esplugues, E., Flavell, R., and Craft, J. (2016). TFH cells progressively differentiate to regulate the germinal center response. Nat. Immunol. 17, 1197-1205.

Wheaton, W. W., Weinberg, S. E., Hamanaka, R. B., Soberanes, S., Sullivan, L. B., Anso, E., Glasauer, A., Dufour, E., Mutlu, G. M., Budigner, G. S., and Chandel, N. S. (2014). Metformin inhibits mitochondrial complex I of cancer cells to reduce tumorigenesis. Elife 3, e02242.

Xin, G., Schauder, D. M., Lainez, B., Weinstein, J. S., Dai, Z., Chen, Y., Esplugues, E., Wen, R., Wang, D., Parish, I. A., et al. (2015). A critical role of IL-21-induced BATF in sustaining CD8-T-cell-mediated chronic viral control. Cell Rep. 13, 1118-1124.

Xin, G., Zander, R., Schauder, D. M., Chen, Y., Weinstein, J. S., Drobyski, W. R., Tarakanova, V., Craft, J., and Cui, W. (2018). Single-cell RNA sequencing unveils an IL-10-producing helper subset that sustains humoral immunity during persistent infection. Nat. Commun. 9, 5037.

Xu, Y., Xue, D., Bankhead, A., 3rd, and Neamati, N. (2020). Why all the fuss about oxidative phosphorylation (OXPHOS)? J. Med. Chem. 63, 14276-14307.

Yarbro, J. W. (1992). Mechanism of action of hydroxyurea. Semin.Oncol. 19, 1-10.

Zhang, H., Xu, Y., Joseph, J., and Kalyanaraman, B. (2005). Intramolecular electron transfer between tyrosyl radical and cysteine residue inhibits tyrosine nitration and induces thiyl radical formation in model peptides treated with myeloperoxidase, H2O2, and NO2-: EPR SPIN trapping studies. J. Biol. Chem. 280, 40684-40698.

Zhou, B., Su, L., Hu, S., Hu, W., Yip, M. L., Wu, J., Gaur, S., Smith, D. L., Yuan, Y. C., Synold, T. W., et al. (2013). A small-molecule blocking ribonucleotide reductase holoenzyme formation inhibits cancer cell growth and overcomes drug resistance. Cancer Res. 73, 6484-6493.

TABLE 1
Key reagents and Resources

| REAGENT or RESOURCE | SOURCE | IDENTIFIER |
|---|---|---|
| Antibodies | | |
| Ly6C | Biolegend | Cat# 128015 |
| Ly6G | Biolegend | Cat# 127613 |
| CD11b | Biolegend | Cat# 101223 |
| F4/80 | Biolegend | Cat# 123113 |
| CD4 | Biolegend | Cat# 100460 |
| CD25 | Biolegend | Cat# 102012 |
| FOXP3 | Invitrogen | Cat# 12-5773-82 |
| LIVE/DEAD ™ Fixable Violet | Invitrogen | Cat# L34964 |
| Dead Cell Stain Kit | | |
| S100A9 | BD Biosciences | Cat# 566010 |
| Chemicals, Peptides, and Recombinant Proteins | | |
| Hydroxyurea | Combi-Blocks | Cat# OR-0532 |
| 1,14-Tetradecanediol | Combi-Blocks | Cat# OR-0896 |
| DMF | Fisher Scientific | Cat# AC348431000 |
| CH₂Cl₂ | Fisher Scientific | Cat# 143SK4 |
| Mito-HU derivatives | This manuscript | N/A |
| 1,10-Dibromodecane | Alfa Aesar | Cat# A12357.22 |
| Sodium sulfate | Sigma-Aldrich | Cat# 238597 |
| Acetonitrile | Sigma-Aldrich | Cat# 34998 |
| Acetic anhydride | Sigma-Aldrich | Cat# 8222781000 |
| HBr | Sigma-Aldrich | Cat# 244260 |
| Pentane | Sigma-Aldrich | Cat# 1071761000 |
| Diethylether | Sigma-Aldrich | Cat# 673811 |
| Bromodecane | Sigma-Aldrich | Cat# 145785 |
| Triethylamine | Sigma-Aldrich | Cat# 90335 |
| EtOH | Sigma-Aldrich | Cat# 1009861000 |
| Carbonyldiimidazole (CDI) | Sigma-Aldrich | Cat# 115533 |
| Potassium carbonate | Sigma-Aldrich | Cat# 209619 |
| Pyruvate | Sigma-Aldrich | Cat# P2256 |
| Malate | Sigma-Aldrich | Cat# M7397 |
| Succinate | Sigma-Aldrich | Cat# S3674 |
| (4-Bromobutyl)-triphenylphosphonium | Sigma-Aldrich | Cat# 272132 |
| bromide | | |
| Triphenylphosphine | Sigma-Aldrich | Cat# T84409 |
| Tri(p-tolyl)phosphine | Sigma-Aldrich | Cat# 287830 |
| Rotenone | Sigma-Aldrich | Cat# R8875 |
| Malonate | Sigma-Aldrich | Cat# M1296 |
| Antimycin A | Sigma-Aldrich | Cat# A8674 |
| 1,16-Hexadecanediol | TCI | Cat# H0552 |
| Eicosanedioic acid | TCI | Cat# E0320 |
| Plasma Membrane Permeabilizer (PMP) | Agilent | Cat# 102504-100 |
| Adenosine 5′-diphosphate monopotassium | Sigma-Aldrich | Cat# A5285 |
| salt dihydrate (ADP) | | |
| Experimental Models: Cell Lines | | |
| Human: MiaPaCa-2 | American Type Culture Collection | Cat# CRL-1420 |
| Human: HCT-116 | American Type Culture Collection | Cat# CCL-247 |
| Other | | |
| RPMI 1640 Medium | Gibco | Cat# 11875093 |
| Dulbecco's Modified Eagle Medium | Gibco | Cat# 11965118 |
| Fetal bovine serum, qualified, USDA- | Omega Scientific, Inc. | Cat# FB-12 |
| approved regions | | |

TABLE 2
IC50 values of Mito-HUs (mean ± SD, μM)

| | Cell | Complex | Complex | | Suppressive |
|---|---|---|---|---|---|
| | Proliferation | I | III | M-MDSC | Neutrophils |
| | (FIG. 1) | (FIG. 2) | (FIG. 2) | (FIG. 6) | (FIG. 6) |
| HU | 180 ± 6.7 | >3000 | >3000 | 84 ± 2.8 | 18 ± 1.9 |
| Mito₄-HU | >600 | N.A. | N.A. | N.A. | N.A. |
| Mito₁₀-HU | 8.5 ± 0.41 | 8.9 ± 1.22 | 21 ± 0.72 | 9.6 ± 0.39 | >20 |
| Mito₁₂-HU | 1.8 ± 0.05 | 1.6 ± 0.16 | 3.1 ± 0.18 | 2.6 ± 0.11 | >4 |
| Mito₁₄-HU | 0.90 ± 0.04 | 1.0 ± 0.12 | 1.7 ± 0.69 | 0.89 ± 0.10 | 0.94 ± 0.99 |
| Mito₁₆-HU | 0.50 ± 0.01 | 0.8 ± 0.05 | 2.6 ± 0.39 | 0.44 ± 0.11 | 0.56 ± 0.12 |
| Mito₂₀-HU | 0.23 ± 0.02 | 0.8 ± 0.06 | 4.2 ± 0.40 | 0.44 ± 0.09 | >1 |

TABLE 3
Effects of HU and Mito-HUs on proliferation of HCT116 human colon cancer
cells; related to FIG. 1. Effects of HU and Mito-HUs on the proliferation of HCT116 cells were
monitored in the IncuCyte Live-Cell Analysis system, which provides real-time updates on cell
confluence based on a segmentation of high-definition-phase- contrast images. Data are
represented as a percentage of cell confluence when control cells reached ~90% confluence
(vertical black dashed line from FIG. 1). Data shown are the means ± SD, n = 4 per treatment group.

| HU | Cell | | Mito₄-HU | Cell | | Mito₁₀-HU | Cell | |
|---|---|---|---|---|---|---|---|---|
| (μM) | Confluence (%) | | (μM) | Confluence (%) | | (μM) | Confluence (%) | |
| 0 | 91.04 | ± 6.49 | 0 | 90.03 | ± 3.49 | 0 | 91.03 | ± 1.81 |
| 10 | 89.91 | ± 7.19 | 100 | 92.40 | ± 1.57 | 0.1 | 82.13 | ± 6.87 |
| 30 | 91.61 | ± 5.07 | 200 | 90.77 | ± 1.82 | 0.3 | 84.69 | ± 4.98 |
| 100 | 66.96 | ± 9.44** | 400 | 80.77 | ± 3.25** | 1 | 83.91 | ± 6.35 |
| 150 | 48.62 | ± 9.11** | 600 | 47.24 | ± 2.47** | 3 | 64.31 | ± 5.85** |
| 200 | 31.90 | ± 5.77** | | | | 5 | 67.01 | ± 5.30** |
| 250 | 22.99 | ± 4.30** | | | | 10 | 26.84 | ± 2.42** |
| 300 | 17.30 | ± 3.35** | | | | 12.5 | 13.75 | ± 1.42** |
| 400 | 11.42 | ± 2.17** | | | | 15 | 8.68 | ± 0.76** |
| 500 | 8.79 | ± 1.41** | | | | 20 | 3.45 | ± 0.44** |
| 1000 | 5.40 | ± 1.02** | | | | 30 | 1.74 | ± 0.12** |
| 3000 | 3.43 | ± 0.90** | | | | 100 | 1.53 | ± 0.11** |

| | | | | | |
|---|---|---|---|---|---|
| Mito₁₂-HU | Cell | Mito₁₄-HU | Cell | Mito₁₆-HU | Cell |
| (μM) | Confluence (%) | (μM) | Confluence (%) | (μM) | Confluence (%) |

* p < 0.05, **p < 0.01 vs control.

TABLE 4
Effects of HU and Mito-HUs on oxygen consumption by mitochondrial complex
I in HCT116 human colon cancer cells; related to FIG. 2A. Effects of HU, Mito10-HU, Mito12-
HU, Mito14-HU, Mito16-HU, and Mito20-HU on complex I-dependent oxygen consumption were
measured in HCT116 cells. Data represent complex I activity (% of control, calculated from FIG.
2A) used to determine the IC50 values in FIG. 2B. Data shown are the means ± SD, n = 4 per treatment group.

| HU | Complex I activity | | Mito₁₀-HU | Complex I activity | | Mito₁₂-HU | Complex I activity | |
|---|---|---|---|---|---|---|---|---|
| (μM) | (OCR, % of control) | | (μM) | (OCR, % of control) | | (μM) | (OCR, % of control) | |
| 0 | 100.00 | ± 8.54 | 0 | 100.00 | ± 6.98 | 0 | 100.00 | ± 1.49 |
| 10 | 84.69 | ± 7.90 | 0.1 | 104.12 | ± 0.13 | 1 | 76.18 | ± 4.73** |
| 30 | 77.03 | ± 1.79** | 1 | 87.41 | ± 3.09* | 1.5 | 60.18 | ± 2.79** |
| 100 | 87.88 | ± 9.53 | 3 | 90.19 | ± 6.64 | 2 | 51.91 | ± 15.01** |
| 150 | 86.10 | ± 3.50* | 5 | 84.25 | ± 7.55* | 2.5 | 33.88 | ± 11.28** |
| 200 | 86.45 | ± 2.07* | 10 | 53.43 | ± 4.96** | 3 | 4.75 | ± 2.20** |
| 250 | 91.32 | ± 1.98 | 12.5 | 38.54 | ± 4.80** | 4 | 1.51 | ± 1.74** |
| 300 | 85.66 | ± 3.16* | 15 | 21.08 | ± 4.73** | 5 | 1.55 | ± 5.50** |
| 400 | 70.49 | ± 7.99** | 20 | 5.51 | ± 10.05** | 7.5 | 2.39 | ± 1.86** |
| 500 | 82.13 | ± 9.12* | 40 | 3.14 | ± 3.67** | 10 | 10.99 | ± 1.09** |
| 1000 | 80.18 | ± 10.83* | 50 | 2.72 | ± 3.40** | | | |
| 3000 | 83.07 | ± 5.77* | | | | | | |
| Mito₁₄-HU | Complex I activity | | Mito₁₆-HU | Complex I activity | | Mito₂₀-HU | Complex I activity | |
| (μM) | (OCR, % of control) | | (μM) | (OCR, % of control) | | (μM) | (OCR, % of control) | |
| 0 | 100.00 | ± 8.18 | 0 | 100.00 | ± 3.64 | 0 | 100.00 | ± 1.51 |
| 0.1 | 84.12 | ± 5.17* | 0.03 | 94.36 | ± 2.33 | 0.03 | 94.62 | ± 11.43 |
| 0.5 | 64.29 | ± 3.13** | 0.1 | 88.91 | ± 5.60* | 0.1 | 101.46 | ± 8.97 |
| 0.75 | 57.90 | ± 5.35** | 0.3 | 81.13 | ± 8.84** | 0.2 | 88.69 | ± 7.81* |
| 1 | 53.52 | ± 3.18** | 0.4 | 69.28 | ± 7.08** | 0.3 | 90.49 | ± 3.66** |
| 1.5 | 47.56 | ± 11.24** | 0.5 | 64.33 | ± 7.02** | 0.5 | 63.56 | ± 7.46** |
| 2 | 27.58 | ± 4.49** | 0.6 | 59.73 | ± 6.65** | 1 | 35.93 | ± 4.82** |
| 4 | 8.63 | ± 5.08** | 0.8 | 45.37 | ± 1.48** | 3 | 8.51 | ± 1.25** |
| 10 | 7.14 | ± 12.62** | 1 | 43.04 | ± 3.82** | 5 | 6.26 | ± 2.75** |
| 20 | 0.11 | ± 2.19** | 3 | 15.78 | ± 4.12** | 10 | 0.00 | ± 1.43** |
| | | | 5 | 9.68 | ± 1.36** | 20 | 0.00 | ± 4.42** |
| | | | 10 | 2.14 | ± 1.33** | | | |

*p < 0.05,
**p < 0.01 vs control.

TABLE 5
Effects of HU and Mito-HUs on oxygen consumption by mitochondrial complex
III in HCT116 human colon cancer cells; related to FIG. 2A. Effects of HU, Mito10-HU, Mito12-
HU, Mito14-HU, Mito16-HU, and Mito20-HU on complex I-dependent oxygen consumption were
measured in HCT116 cells. Data represent complex III activity (% of control, calculated from FIG.
2A) used to determine the IC50 values in FIG. 2B. Data shown are the means ± SD, n = 4 per treatment group.

| HU | Complex III activity | | Mito₁₀-HU | Complex III activity | | Mito₁₂-HU | Complex III activity | |
|---|---|---|---|---|---|---|---|---|
| (μM) | (OCR, % of control) | | (μM) | (OCR, % of control) | | (μM) | (OCR, % of control) | |
| 0.000001 | 100.00 | ± 4.98 | 0 | 100.00 | ± 7.32 | 0 | 100.00 | ± 3.60 |
| 10 | 94.10 | ± 5.76 | 0.1 | 101.00 | ± 3.30 | 1 | 92.06 | ± 11.08 |
| 30 | 81.22 | ± 3.91* | 1 | 95.23 | ± 7.14 | 1.5 | 74.02 | ± 2.00** |
| 100 | 94.61 | ± 7.97 | 3 | 97.84 | ± 5.05 | 2 | 55.15 | ± 14.80** |
| 150 | 92.25 | ± 10.44 | 5 | 99.95 | ± 5.65 | 2.5 | 51.26 | ± 4.32** |
| 200 | 82.70 | ± 3.74** | 10 | 79.48 | ± 7.75** | 3 | 35.88 | ± 11.53** |
| 250 | 89.14 | ± 9.82 | 12.5 | 74.10 | ± 7.58** | 4 | 35.02 | ± 16.17** |
| 300 | 88.71 | ± 4.83* | 15 | 64.34 | ± 8.25** | 5 | 32.51 | ± 2.39** |
| 400 | 86.78 | ± 7.03* | 20 | 52.07 | ± 4.24** | 7.5 | 24.56 | ± 2.19** |
| 500 | 88.88 | ± 7.09* | 40 | 35.00 | ± 9.26** | 10 | 23.56 | ± 2.42** |
| 1000 | 93.09 | ± 5.34 | 50 | 36.78 | ± 3.94** | | | |
| 3000 | 94.37 | ± 3.28 | | | | | | |
| Mito₁₄-HU | Complex III activity | | Mito₁₆-HU | Complex III activity | | Mito₂₀-HU | Complex III activity | |
| (μM) | (OCR, % of control) | | (μM) | (OCR, % of control) | | (μM) | (OCR, % of control) | |
| 0 | 100.00 | ± 7.34 | 0 | 100.00 | ± 4.72 | 0 | 100.00 | ± 11.19 |
| 0.1 | 83.86 | ± 10.03 | 0.03 | 94.04 | ± 1.67 | 0.03 | 98.80 | ± 3.60 |
| 0.5 | 76.36 | ± 3.01** | 0.1 | 89.36 | ± 2.34** | 0.1 | 106.63 | ± 2.40 |
| 0.75 | 72.80 | ± 3.66** | 0.3 | 94.20 | ± 2.64 | 0.2 | 101.11 | ± 6.18 |
| 1 | 67.50 | ± 5.85** | 0.4 | 81.38 | ± 1.88** | 0.3 | 107.99 | ± 4.66 |
| 1.5 | 61.97 | ± 10.33** | 0.5 | 79.37 | ± 4.86** | 0.5 | 102.43 | ± 6.90 |
| 2 | 35.09 | ± 5.20** | 0.6 | 80.12 | ± 5.81** | 1 | 96.57 | ± 4.65 |
| 4 | 29.81 | ± 2.44** | 0.8 | 68.47 | ± 0.97** | 3 | 60.27 | ± 4.43** |
| 6 | 22.89 | ± 5.25** | 1 | 64.66 | ± 3.05** | 5 | 49.04 | ± 5.45** |
| 10 | 13.87 | ± 5.27** | 3 | 63.15 | ± 5.98** | 10 | 26.59 | ± 10.00** |
| 20 | 1.63 | ± 4.97** | 5 | 36.41 | ± 5.29** | 20 | 12.77 | ± 3.11** |
| | | | 10 | 25.51 | ± 1.69** | | | |

*p < 0.05,
**p < 0.01 vs control.

TABLE 6
Mito-HUs Concentration Tested (in FIG. 4)
With Maximum Effect on IFNγ-YFP

| | Concentration with Maximum | |
|---|---|---|
| Derivative | Effect on IFN -YFP | |
| HU | 200 | μM |
| Mito₁₀-HU | 12.5 | μM |
| Mito₁₂-HU | 10 | μM |
| Mito₁₄-HU | 1.5 | μM |
| Mito₁₆-HU | 0.4 | μM |
| Mito₂₀-HU | 0.3 | μM |

### Example 2—Mito-HU Sensitizes Colon Cancer cells to AZD-3965

AZD-3965 is a monocarboxylate transporter 1 (MCT-1) inhibitor and inhibits lactate transport in cancer cells. AZD-3965 is currently undergoing phase 1/phase 2 clinical trials for cancer treatment. The selective inhibition of lactate transport by AZD-3965 presents a novel way to target a metabolic phenotype in tumors overexpressing MCT-1 transporter. However, other cells (skeletal muscle cells and neuronal cells) also need lactate and, at the high concentrations typically used to inhibit tumor proliferation, AZD-3965 elicits toxicity. We show here that the combined use of Mito₁₀-HU and AZD-3965 can overcome the toxic side effects of AZD-3965 and enhance the therapeutic efficacy. FIG. 35 shows that proliferation of colon cancer cells (HCT-116) treated with a combination of AZD-3965 and Mito₁₀-HU are synergistically inhibited. However, independently, these drugs at the concentrations used show negligible antiproliferative effects.

1. A mito-hydroxyurea compound of one of the formulas: 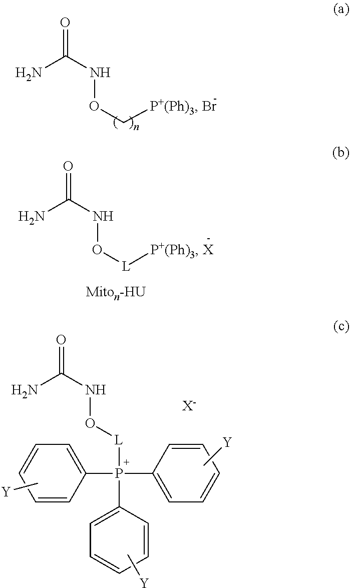
wherein n is an integer from 1-20, L is a linker, wherein the linker is selected from an unsubstituted C₁-C₂₀ alkyl, substituted C₁-C₂₀ alkyl, C₂-C₂₀alkenyl, phenyl, phenyl substituted C₁-C₂₀ alkyl, cycloalkyl, substituted C₁-C₂₀ alkyl, an amino acid, and polyethylene glycol (PEG);
each Y is independently selected from —H, —CF₃, methyl (Me), Cl, OMe, C(O)CH₃, NO₂, N(Me)₂;
and
X⁻ is selected from halogen, 2,2,2-trifluoroacetic acid (TFA), HO⁻, RCOO⁻, and acetic acid.

2. The mito-hydroxyurea compound of claim 1, wherein the compound is (c), wherein the compound is 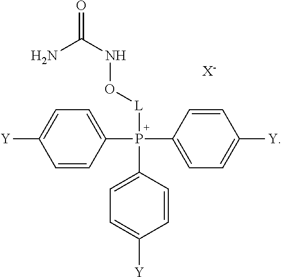
wherein the Y is para.

3. The mito-hydroxyurea compound of claim 1, wherein n is 4-20.

4. (canceled)

5. (canceled)

6. (canceled)

7. The mito-hydroxyurea compound of claim 1, wherein the compound is selected 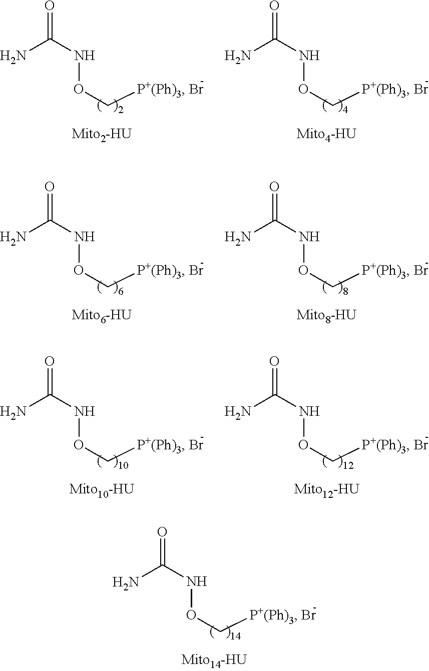
from:

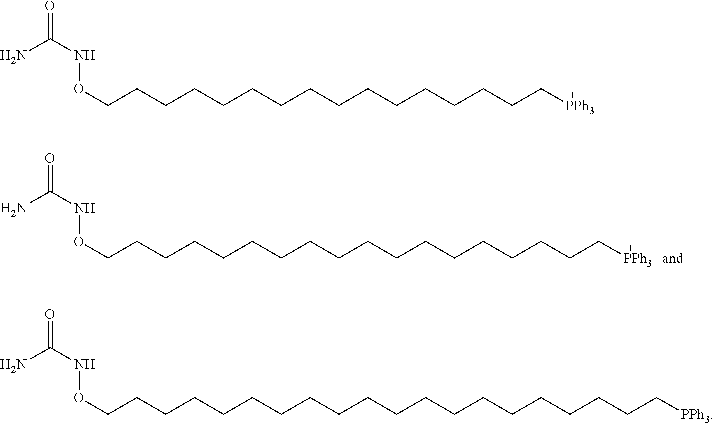

8. A composition comprising the mito-hydroxyurea compound of claim 1 and a pharmaceutically acceptable carrier.

9. A method of treating cancer in a subject in need thereof, the method comprising administering a therapeutically effective amount of the compound of claim 1 to the subject to treat the cancer.

10. The method of claim 9, wherein a therapeutically effective amount of a monocarboxylate transporter 1 (MCT-1) inhibitor is also administered to the subject.

11. The method of claim 10, wherein the MCT-1 inhibitor is selected from AZD-3965 and syrosingopine.

12. The method of claim 9, wherein the cancer is colon cancer or pancreatic cancer.

13. (canceled)

14. A method of inhibiting mitochondrial complex I and complex III in a subject in need thereof, the method comprising administering a therapeutically effective amount of the compound of claim 1 to the subject in order to inhibit, mitochondrial complex I, III or both in a subject.

15. (canceled)

16. A method of increasing an immune response to cancer cells in a subject in need thereof, the method comprising administering a therapeutically effective amount of the compound of claim 1 to the subject in order to increase an immune response to the cancer cells.

17. (canceled)

18. A method of reducing the number of immunosuppressive cells in a subject in need thereof, the method comprising administering a therapeutically effective amount of the compound of claim 1 to the subject in order to reduce the number of immunosuppressive cells in the subject.

19. The method of claim 18, wherein the immunosuppressive cells are T regulatory cells (T_{regs}) or myeloid derived suppressor cells (MDSCs).

20. (canceled)

21. A method of potentiating radiation therapy in a subject in need thereof, the method comprising administering a compound of claim 1 to a subject and treating the subject with radiation therapy.

22. (canceled)

23. A method of inhibiting proliferation of a cancer cell, the method comprising contacting a cancer cell with an effective amount of claim 1.

24. (canceled)

25. The method of claim 23, wherein an effective amount of the compound is a concentration of less than about 20 μM.

26. The method of claim 23, wherein an effective amount of the compound is a concentration of less than about 10 μM.

27. A method of increasing the production of interferon-γ (IFN-γ) in a T cell, the method comprising contacting the cell with an effective amount of the compound of claim 1.

28. (canceled)

29. The method of claim 27, wherein an effective amount of the compound is a concentration of less than about 20 μM.

30. The method of claim 27, wherein an effective amount of the compound is a concentration of less than about 10 μM.
